SEQUENCE LISTING (115 sequences)

SEQ ID NO: 1 - protein, 399 aa, Kitasatospora aureofaciens
MDVLAALERKHSLNLFPIENRLSPRAAAALASDAVNRYPYSETDVAVYGDVSDLNAVYDHCVSLTKEFYGARHAYVQFLSGLHTMHTVLTAVTPPGGRVMVIAPEDGGHYATVTICQGFGYRVEYVPFDRQTLEIDYTALAERTAEHPADVIYLDASTVLRMPDARALRAAAPGAVLCLDASHLLGLLPAAPGTLVLDAGFDSISGSTHKTLPGPQKGLLVTNSDAIAEQVGARIPFTASSSHSASVGSLAITLEELLPHRGDYARQVIANARELARQLAARGFDVAGEAFGFTDTHQVWVHHPEGNTPHEWGRLLTATDIRTTTVVLPSTARSGLRLGTQELTRWGMKEDDMTTVAELLARLLLRGEQSRSVAADVRDLARSFPGVAFADRPAPLAVA

SEQ ID NO: 2 - protein, 414 aa, Streptomyces clavuligerus
MKSVRRRRSPSDSVPFRPPIRGESMDVLAALERKPSLNLFPIENRLSPRASAALATDAVNRYPYSETPVAVYGDVTGLAEVYAYCEDLAKRFFGARHAGVQFLSGLHTMHTVLTALTPPGGRVLVLAPEDGGHYATVTICRGFGYEVEFLPFDRRTLEIDYAVLAARLSRRPADVIYLDASSILRFIDARALRLAAPDALICLDASHILGLLPVAPQTLVLDGGFDSISGSTHKTFPGPQKGLLVTDSDVVAEKVAARMPFTASSSHSASVGSLAISLEELLPHRTAYAHQVIANARALAGLLAERGFDVAGGAFGHTDTHQVWVHFPEGNTPHEWGRLLTRANIRSTSVVLPSSAAPGLRLGTQELTRWGMTETDMAPVADLLERLLLRGDDAETVAKEVVELARAFPGVAFV

SEQ ID NO: 3 - protein, 416 aa, Streptomyces antibioticus
MKESPPVPPRPSQECPMDVLEVLRRKPSLNLFPIENRLSPRAREALASDANNRYPYVEGPVSHYGDVMGLGEVYDYCVDLAKEFYGARHGCVHFLSGLHTMYTVITALVPAGSRVMVLHPEDGGHYATITICEGLGHSVSRLPFDRKTLLIDYEELAVQLAESPVDVIYLDASSMLRLPDARLLRQAAPDTLLCLDASHLMGILPAAPKTLVFDGGFDTVSGSTHKTLPGPQKGLMVTNDATLAGKVMERIPFTASSSHAGNVGALAITLEELMPCRVEHAQQIIANARELAAQLAQRGFSVAGEEFGWTETHQVWAYIPEEQGPHGWGRVLTRANVRSTTVPLPSSDGLPALRLGTQELTRSGMKEAEMTEVADILERLLLRGEAPEQVIGTVRDLALRFPGVSWIGSADTTSVD

SEQ ID NO: 4 - protein, 390 aa, Streptomyces clavuligerus
MDVLAALERKPSLNLFPIENRLSPRASAALATDAVNRYPYSETPVAVYGDVTGLAEVYAYCEDLAKRFFGARHAGVQFLSGLHTMHTVLTALTPPGGRVLVLAPEDGGHYATVTICRGFGYEVEFLPFDRRTLEIDYAVLAARLSRRPADVIYLDASSILRFIDARALRLAAPDALICLDASHILGLLPVAPQTLVLDGGFDSISGSTHKTFPGPQKGLLVTDSDVVAEKVAARMPFTASSSHSASVGSLAISLEELLPHRTAYAHQVIANARALAGLLAERGFDVAGGAFGHTDTHQVWVHFPEGNTPHEWGRLLTRANIRSTSVVLPSSAAPGLRLGTQELTRWGMTETDMAPVADLLERLLLRGDDAETVAKEVVELARAFPGVAFV

SEQ ID NO: 5 - protein, 399 aa, Kitasatospora sp. MBT63
MDVLAALERKHSLNLFPIENRLSPRAAAALASDAVNRYPYSETDVAVYGDVSGLNGVYDYCVSLTKEFYGARHAYVQFLSGLHTMHTVLTAVTPPGGRVMVLAPDDGGHYATVTICRGFGYQVEFVPFDRQALEIDYAALAERTAEQRVDVIYLDASTVLRMPDARALRAAAPDAVLCLDASHLLGLLPAAPDTLVLDGGFDSISGSTHKTLPGPQKGLLVTNSDAIAEQVGARIPFTASSSHSASVGSLAITLEELLPYREEYPRQVIANARELGRQLAARGFDVAGGKFGHTDTHQVWVHHPEGNTPHEWGRLLTATDIRTTTVVLPSSARSGLRLGTQELTRWGMKEQDMATVAELLERLLLRGEKSASVAADVQDLARSFPGVAFAGRPVPLAVA

SEQ ID NO: 6 - protein, 399 aa, Streptomyces aurantiacus
MDVLATLRRQPSLNLFPIENRLSPRALEALSSDANNRYPYSETDVAVYGDVTGLNDVFTYCTDLTKQFYGARHAYVNFLSGLHTMHTVITAVATAGDRVMVLAPEDGGHYATATICRGYGHEVDFLPFDRGTLEIDYAKLATTVAERPVDLIYLDASSMLRFPDARALRAAAPDALICLDASHLLGLLPVAPQTLVLDGGFDSISGSTHKTMPGPQKGLLVTNSDRMAELVGARIPFTASSSHSASVGSLAITLEELMPHRTAYAQQVIDNARALGSQLASRGFDVAGKDFGYSETHQVWVHLPDGHTTHQWGRTLTAAGIRSTTVQLPSTGRPGLRLGTQELTRWGMRESDMSVVADLLARLLLRGEAVKEIAEDVSTLALSYPGVAFAGPLAPLASR

SEQ ID NO: 7 - protein, 399 aa, Streptomyces sp. 3214.6
MDVLATLRQKPSLNLFPIENRLSPRALEALATDANNRYPYSETPVAVYGDVTGLNDVYEYCVELTKRFYGARHGFVNFLSGLHTMHTVITAVARPGDRVMLLAPEDGGHYATDTICAGYGYEREFLPFDRAAMEIDYAKLAVRVAERPVDLIYLDASSTLRFPDARALRAAAPDALICLDASHLLGLLPVAPQTLVLDGGFDSISGSTHKTLPGPQKGLLVTNSDTMADKVAARIPYTASSSHSANVGALAVTLEELLPHRAAYAQQVIANARALGRELAGRGFDVAGASFGHTDTHQVWVQFPEGNTPHEWGRTLTAAAIRTTTVVLPSNAQPGLRLGTQELTRWGMREQDMSAVAELLARLLLRGESVESVTGDVAELALSFPGVAFAGALEPVTAP

SEQ ID NO: 8 - protein, 387 aa, Salinispora pacifica
MFPIENRLSPRAGMALSSDATNRYPYVEGALTHYGDVSGLNDVYAYCVDLARKYLGGRYGCVHFLSGLHTMYTVITALVPPGSRIMALDPEDGGHYATVTICEGLGHKMSFLPFDRERLLIDYERLADQLRQEPVDVIYVDASSMLRFPDARALRAAAPDTLLLLDASHLMGLLPAAPQTGVLDGGFDIIQGSTHKTMPGPQKGLMVTNHEELVRKVEARVPYTASSSHAANVGALAITLEELLPCRLSYARQVIANARELAGQLAGRGFGVAGEAFGWTDTHQVWLDIPAEIGPHRWGRLLTQANVRSTTVPLPSSGGLPALRLGTQELTRVGMEEQEMAEVASILDRILLRGENPDSVVETVTKLVTRFPEVKFIGKPGEDESFS

SEQ ID NO: 9 - protein, 399 aa, unclassified Streptomyces
MDVLAALQRRPSLNLFPIENRLSPRAAAALATDAVNRYPYSETPVAVYGDVTGLKDVYDYCADLTKEFYGARHAFVPFLSGLHTMHTVLTAVAPPGGRVMVLAPDDGGHYATVTICEGFGYEVDYLPFDRQRLEIDHAALAVRTAERPVDVIYLDASTALRFPDARALRAAAPGAILCLDASHLLGLLPAAPQTLVLDGGFDSISGSTHKTLPGPQKGLLVTNSDSLAEKMAARIPFTASSSHSATVGSLAITLEELMPHRVEYAQQIIANARRLAGELAGLGFDVAGEEFGHTDTHQVWVHPPEGNTPHEWGRLLTRTDIRTTTVVLPSSRSSGLRLGAQELTRWGMKENDMARVAELLARLLLHHEDSGKVAADVADLARAFPGVAYAGGSAAVTAG

SEQ ID NO: 10 - protein, 400 aa, Streptomyces
MDVLAALRRRPSLNLFPIENRLSPRAREALASDAGNRYPYVEGPVTHYGDVMGLSEVYDYCVDLTRRFYGARFGCVHFLSGLHTMYTVITALARPGSRVMVLDPEDGGHYATVTICEGLGYSVSRLPFDRQRLLIDYDALAVRMRERPVDLVYLDASSMLRFPDARLLRQAAPDALLCLDASHLLGLLPAAPRTLVFGGGFDTISGSTHKTLPGPQKGLLVTDNEALARRVRERVPFTASSSHAASVGALAITLEELMPCRVAHAEQIIANARELASQLAQRGFGVAGEGFGWTETHQVWVHIPEEAGPHGWGRLLTRADIRSTTVPLPSSAGLPALRLGTQELTRCGMKEDTMAEVAGLLARVLLRGEAPEAVVADVRALAERFPGVAYVGTPEVTVEE

SEQ ID NO: 11 - protein, 400 aa, Streptomyces sp. RP5T
MDVLEVLRRKPSLNLFPIENRLSPRAREALASDANNRYPYVEGPVSHYGDVMGLGEVYDYCVDLAKEFYGARHGCVHFLSGLHTMYTVITALVPAGSRVMVLHPEDGGHYATITICEGLGHSVSRLPFDRKTLLIDYEELAARLAESPVDVIYLDASSMLRLPDARLLRQAAPDTLLCLDASHLMGILPAAPKTLVFDGGFDTVSGSTHKTLPGPQKGLMVTNDATLAGKVMERIPFTASSSHAGNVGALAITLEELMPCRVEHAQQIIANARELAAQLAQRGFSVAGEEFGWTETHQVWAYIPEEQGPHGWGRVLTRANVRSTTVPLPSSDGLPALRLGTQELTRSGMKEAEMTEVADILERLLLRGEAPEQVIGTVRDLALRFPGVSWIGSADTTSVD

SEQ ID NO: 12 - protein, 400 aa, Streptomyces sp. uw30
MDVLEVLRRQPSLNLFPIENRLSPRAREALSSDANNRYPYVEGPVSHYGDVMGLDKVYDYCVELAKEFYGARYGCVHFLSGLHTMYTAITALVPPRSRVMVLHPEDGGHYATITVCEGLGHSISRLPFDRKNLLIDYDKLAAELEENPVDAIYLDASSMLRLPDARLLRQAAPDVLMCLDASHLLGILPAAPQTLVLDGGFDTISGSTHKTLPGPQKGLLVTNDEALAQKVVERIPFTASSSHAGSVGALAVTLEELLPCRVEHAEQIVSNARELAAQLAGRGFSVAGEEFGWTQTHQVWAYIPEEQGPHGWGRLLTEANIRSTTVPLPSSDGLPALRLGTQELTRSGMKEADMAEVAEILERILLRGEAPERVAGQVRDLALRFPGVAYIGSPQGMSAD

SEQ ID NO: 13 - protein, 399 aa, Streptomyces sp. SID10853
MDVLAALQQRPSLNLFPIENRLSPRAAAALATDAVNRYPYSETPVAVYGDVAGLSDVYDYCVDLTKEFYGARHAFVQFLSGLHTMHTVLTAVTPRSGRVMVLAPEDGGHYATVTICESFGYRADYIPYDRKRLQIDHSALAARIAEQPVDVIYLDASTTLHFPDARALRAAAPDAIICLDASHLLGLLPAAPQTLVLDGGFDSISGSTHKTLPGPQKGLFVTNSDTVAEKVAARIPFTASSSHSATVGSLAITLEELLPHRVDYARQTIANARRLGEELARRGFDLPGEDFGYTDTHQVWVHPPEECSPHEWGRALTRADIRTTTVGLPSSGRSGLRLGSQELTRWGMKEADMAAVAELLARLLLRGDDTGRVAADVADLAREFPGVAYAGQPAPVTVT

SEQ ID NO: 14 - protein, 397 aa, Streptomyces sp. DSM 110735
MTPEEIIHRFGRVSPTLNLYPIENRLSDGARSLLGSDLVSRYPRMSGPGYLYGDPSNVADLYEECAALACEYFQVDHALVHFLSGLHAMQSMISTLSEPGERIVSLGPDAGGHYATEQICRDFGHDTGLLPFDGVNLRVDMDRLAEQHRAAPSRFYYVDLSTALRVPDMEQMRNAVGGDALITFDASHILGLLPVLYDLPALWRQISLCTASTHKTFPGPQKAVMLSSDEKVVADMSEHLKFRVSSAHTNSVGALAVTFSELMDSRRTYARAVIDNARRLAELLSERGLRVVGEHFGFTETHQIWVLPPEGTQDPVDWGARLQSCGIRASVVHLPAQGTSGLRLGTQELTRMGMDPAAMTEVADLTVRALGGGDPELIRKEVADLTARYATVRNDFA

SEQ ID NO: 15 - protein, 385 aa, Streptomyces sp. DSM 110735
MSPTLNLYPIENRLSDGARSLLGSDLVSRYPRMSGPGYLYGDPSNVADLYEECAALACEYFQVDHALVHFLSGLHAMQSMISTLSEPGERIVSLGPDAGGHYATEQICRDFGHDTGLLPFDGVNLRVDMDRLAEQHRAAPSRFYYVDLSTALRVPDMEQMRNAVGGDALITFDASHILGLLPVLYDLPALWRQISLCTASTHKTFPGPQKAVMLSSDEKVVADMSEHLKFRVSSAHTNSVGALAVTFSELMDSRRTYARAVIDNARRLAELLSERGLRVVGEHFGFTETHQIWVLPPEGTQDPVDWGARLQSCGIRASVVHLPAQGTSGLRLGTQELTRMGMDPAAMTEVADLTVRALGGGDPELIRKEVADLTARYATVRNDFA

SEQ ID NO: 16 - protein, 424 aa, Parachlamydiales bacterium
METSLKDFETILHLINKEEIDSNDTIHMTANENIMSKLSKHYLKSTLSYRYHVGMFDDQKNLTVSRSCLIKNSLMLRCLSPIFLLEQQAREYVKKMFFAEYADFRPLSGMHTVFCILSTLTKPNDRVYVFTTESVGHAATVSLLKSLGRKVSFIPFCEKKLDIDLEKLSKQILIEKPNAILFDFGTPFYPLPIREIREIVGNDVKMIYDASHVLGLIAGGQFQNPLLEGCDVLIGNTHKTFPGPQKGMILYKNKSLGKEIATEIFKSAISAQHTHHAIALYVTIIEMYIHGKEYANQIIKNNHALSQALINEGFKIFKRKNQFSLSHMIAITGDFPIDHHVACADLHNSNISTNSRILYDFPAVRIGVQEVTRKGMKEKDMVQLAKFFKEIILDRKNISSKIKEFNNKFNSIEYSLDEIYEKLF

SEQ ID NO: 17 - protein, 443 aa, Streptomyces noursei
MTELAAAGPVRSPHRAGGRTGPAGGLLTAVHDDVGRLTTTVNLAAFENVLSRTARAMLHGPLADRYLIGHEQERRGLDPLLRSGLLSAAYPGVDALERAASETARQLFGAAWVDFRPLSGLHATISVFALLTAPGSTVYSIAPANGGHFATQPLLESMGRDGRYLPWCASAGTVDLAAFAEVWRAHPGAMVFLDHGVPLAPLPVRELRAVIGDGTLLAYDASHTLGLIAGGRFQDPLAEGCDLLQGNTHKSFPGAHKGLVAFADAALGQGFSERLGLALVSSQQTGPTLANYVTTLEMGVHASAYTRQMLANQAALACALGESGFAVHHPPGATGPSASHVLLVEGGRQHDGADPYALAARLMHCGVMLNARPVDGRVVLRLGVQEVTRRGMRQPEMWRLAELMARAAHTEGATATADVAGQVAALAGAFTSVRYGFDDSEAA

SEQ ID NO: 18 - protein, 427 aa, Pseudomonas aeruginosa
MGNSILELLSAEEQKCRSMLHLTSYENRMSKTAEAFLSSDLGNRYHLSTPDTHNGLDPSVHIAGFSCRALSAVHRLELSAIASAKKMFNAAHIEMRLVSGVHATISTIASMTKPGDIVYSIAPEDGGHFATKHVAESLGRKSRYLSWDSERLNVDLEESKALFAMFPPAMVFLDHGTPLFNLPVGELRDLIPSDSLLVYDASHTLGLIAGGYFQHPLCEGADILQGNTHKTFPGPQKAMVMFSSPELGSRYSKSVSLGLVSSQHTHHSIALGVTILEMEAFGAKYAQCMLENAQVLGNALIAEGLGLVSHSGKFTTSHELLINSGWPDGYLSAVDRLFDANISVNGRVAFRRPTIRLGVQEITRRGMGPDEMLVIAKLIAAAVQETDSAESIRLRVDQLNRDFPSTLYSFDHSCSVDSGEELQNAYS

SEQ ID NO: 19 - protein, 445 aa, Gammaproteobacteria bacterium CG11_big_fil_rev_8_21_14_0_20_46_22
MFLNNEISEKLHKLTDLYKYDALFHSLICEEWRDELTLNLCAYDNILSKSARYFLQSQLGFRYRLGEIAKAPVNADYQQKGSLLYTEKPALTQLETKAYDVAVKIFSGIGADFRPLSGVHATMCSVLALTSVNEVVYSIDPGDGGHFATRGVVEMSGRKSVYMPWDRERQDVDFNRLREMLNESKPTLIILEHGCPQRPLNIKRLRETVGDSVFIAYDASHTLGLMAGGLFQSPLLEGCDLLQANTHKSFPGPQKALYIFANSLVQERLSSALDDALVSSQHTHNLMALCISMLEMELWGKEYAIKMLENSAALKNELLKLGFNVLYPNDHSTHIILIEFKDEFSGKAFFQRLLASGIATNFRLMRDKAVIRLGTQELTRKGFEPYQMVYIADLMARANEGERGSHGVASEVSELMRNSNEVHYSFDDNLSINRLIQGNYDASQH

SEQ ID NO: 20 - protein, 422 aa, Frankia elaeagni
MIEIALRELVDDLRAEEGTLARTVHLTPNENVLSRLARSFLSSPIGFRYHLGTISSRRALDGVVDVHGLTLGYLKAVAETEQRAVGAAQGMFDAAIADLRPLSGVHAMITTLSAVTEPGDTVYSIDPACGGHFATRHILQRLGRVSEYLPWDLEALTIDVPRSGEAFLRTQPKAVLLDHGAPLYPLPVQALRESCPSRTVLIYDGSHVLGLIAGAKFQRPLADGCDILQGNMHKSFPGPQKALICAREGVIGESVVDNLSRGFVSSQHTHQSVAAYVTLLEMEKYGQAYAVQMLSNSRSLATSLKAAGFSLVESADTPSESHQILVRTDGQDESIRWVRRLLQCGISVNARRLYGHDVLRVAVQEVTRLGMIESDMEHIAEIFRTALKGKTSASVLRSECISMGRRFSRVLFSFDEHFEPVE

SEQ ID NO: 21 - protein, 425 aa, [Flexibacter] sp. ATCC 35208
MIEQYIETDKEIGRLVTQLVEKEELLNTHVLHLTANENRLSKTAREVLSSALSFRYHLGIPADYNFDDIVAKPNLLFRGLPNLYRLEDMAHRCLNKHLGGVVSDSRPLSGLHAMICSISSLTSPGDIVLSICPEGGGHFATATLINQLGRKSVLIDYDRKTLALSLSHLHQLSKEYNVKAVFLDDSAPLYAMPLKEIRDILGPDVIVIYDASHTLGLIYGQQFLHPLQDGCDVIQANTHKTFPGPQKGLLHFADNTIAGKAMQTIGSCLVSSQHTHHSLAFYITALEMDLHAKNYADMIVANAKLLSGALEKNGFQVLTNGKSFTDTHQILFNLPGHLSHYEISRKLLECHISTNAKHVYERDVVRIGVQELTRLGMRGTEMEEIAGIIKLAVLDDKKEIAVGMVNELNNAFQDVHYSFDNASML

SEQ ID NO: 22 - protein, 423 aa, Flavobacterium pectinovorum
MNSREIEQLIKEEENNLNSFLHLTANENVISEFVSQGLSGTFSNRYHLGQIDKFSDDDITYSNGNIYKGISAINKLERITSIILNNRLGGVDTDFRPLSGVHAMMCTILAVTEVNDYVLTVDPATGGHFATQNIIERTGRKALTVPLNRETLTLDYDFIAKMKDREKIKMFYIDDSFAFQPINFPLLKEILGQNTIIVYDASHPFGFIFAQQFMKPILEGCDILQANTHKIFPGPQKGIIHFANKALASKVKEEIGKSLVSSQHSHHTLALHLAILEMDEFCEAYAEKIIKNTRYLYNSLVEKGFSILEPFQKRELLTNQLYIKVPDGQNAEGIAQRFYSNNISINIRRIFDQTFLRIGLQEVTRLGFNEKEMDELAIIIEDVMFSRNKINISKSVENFELQERKMLFCYQVSKFSEEKLLVE

SEQ ID NO: 23 - protein, 453 aa, Streptomyces cinnamoneus
MTHLAVIDTARPPARPPLRTEPPHALLAAVTDDAARLGSTVNLAAFENVLSRTARAQLAGPLADRYLIGQEHERGLRHPLVRAGLLSAGYPGVDRLESAAVDTLTGLLGAGWADFRPLSGLHATTCTFALLTEPGELVYSIAPDNGGHFATRPLLHSLGRRCAYLPWDAAAGTVDLAGLAAAWRSDPGAMVFLDHGVPLVPLPVAGLRAVTGTGPLLVYDASHTLGLIVGGAFQDPLGEGCDIVQGNTHKSFPGAHKGVIVFADAEAGRRFSERMGGALVSSQQTGATLANYVTALEMGVHAPAYARQMLANRAALAYALREAGFAVHRPAGADAESRSHVLLVDGAGDRFGYELADDLVRAGIVLNARPVEGRIRLRLGVQEVTRRGMRQREMERLADLMARAARGRLPGRGRKAVTVRVRTLAETFGRVHYAFDDIHESHGTTHDGTEAAP

SEQ ID NO: 24 - protein, 444 aa, Streptomyces sp. 769
MTELAAAGPVRSPHRAGGRTDPAGGLLTAVHDDVGRLTTTVNLAAFENVLSRTARAMLHGPLADRYLIGHEQERRGLDPLLRSGLLSAAYPGVDALERAASDTARQLLGAAWVDFRPLSGLHATISVFALLTAPGSTVYSIAPANGGHFATQPLLESMGRDGRYLPWCASAGTVDLAAFAQVWRAHPGAMVFLDHGVPLAPLPVRELRAVIGDGALLAYDASHTLGLIAGGRFQDPLAEGCDLLQGNTHKSFPGAHKGLVAFADAALGQGFSERLGLALVSSQQTGPTLANYVTTLEMGVHASAYTRQMLANQAALACALGESGFVVHHPPGATGPSASHVLLVEGGRQHDDADPYALAARLMHCGVMLNARPVDGRVVLRLGVQEVTRRGMRQPEMWRLAELMARAAHTEGATATAHVAGQVAALAGAFTSVRYGFDDSEAAC

SEQ ID NO: 25 - protein, 419 aa, Leptolyngbya sp. SIOISBB
MIPDKLNALINGIREEEFLSNSVLHLTANENCLSKLASSFLSYSIGSRYALGKSSDRNAEGTWQFGRLTYRGMPSLHHLEEEANQIAYKLFNSTYADFRPLSGVHATICTISTLTKAGDLIFSLPPESGGHFASPQIIHSLGRRNSFLPWNKQKFDIDPDRLEILYRQENPSAILLDYNSPLFPLNLAQIRQIVGEHIPIIYDASHVAGLISGGRFQQPLNDGCTVLQANTHKSFPGPQKGMIHTVQPETAHQISSALSAGLISSQQTNNLIALYITLLEMHENAKAYAKNMILNSEVLAHNLDKQGFKLVNRQNKPSASHILLVEVDSQKKARQWAKKLIESGISVNARRLYGKAVLRLGIQEVTRRGMTTTEMAEIAILFRNAIFDKRSCEELQQEVEELMSHFPHVHYSFDNLTAN

SEQ ID NO: 26 - protein, 398 aa, Saccharothrix sp.
MTAYESKPSRLVQMLSASPLAVDYHIGSLKDHGTDDVVTAHGLVLRGLPGVARLEAEAAGFARRALNAREVDFRPLSGVHAILATLIALTEPGDLVLSISPEHGGHFATRYLLRRIGRRSAYLPWDAEAYAVDVERLAARLSARPAPAAVLFDHGLPLTRQPVERIREVVGERALVLYDASHTLGLVVGRRFQDPLGEGADVVQGNTHKSFPGVQKAVIATRSEELGERIGSALSDGLVSSQHTHHAVATYAAFLEMREFGEGYAEAMIANARALAAELEALGARVIGPAGRWTDSHEVFVAPGAGLAAATWAERLIRAGVSVNARRVHGQDALRIGVQSVTRAGMTTAEMASIARVLTWFLHAERPRAHQSSLIRALTGDFSSVYCSFDHSLGLSAA

SEQ ID NO: 27 - protein, 428 aa, Deltaproteobacteria bacterium
MLSIAQKSSPVFDELKFHLEGIKKQEQQDREILNLNAYDNRVSKTVLSLLSSNLSQRYDLGTPDTHGCSDPAGMGEFLFKGLPHLYKFEQAAITAASLMFGSVTSDFRPLSGMHGMICTLATLTEPDDVVYSVECDYGGHFATHHVLKRLGRRPESIPVDINSLSLDLEAFEKKVRRIPPRLVYLDVGCALYPLPIQDIRRIVGDETIIVYDASHTQGLIAGGVFQMPLAEGADILQGNTHKTFPGPQKAMVHFADYKIAKKLADSLTMGLVSSRHTHHSMALYVTLFEMLEFGGQYARQTLKNATALGKKLKSSGIGLLERDGICTQSNVLLINGKTVGGHVDACRRLYAANIATNSRHAFGKEVIRIGVQELTRRGMNELEMDVIGGFIKRVIVDKEDPFWIKREVMDFNSLFEDVHYSFDAALGY

SEQ ID NO: 28 - protein, 425 aa, Rickettsiales bacterium
MNCIDSSKNLLLKLQNEEKRNTATLHMTANENVMSNTASSFLSSNLSYRYYSDTYEKEDNLAEAKYYAVGQAMYRGLPSVYEFELLARREANKMFHANFSDFCPLSGMNAVICILTTITKPGDKVFIFTPESLGHHATKIVLQNIGREVLFIPWDNEKLCIDIESFEEEFSKNNAATIFLDLGTTFYPLPLKKIRQIVGTRTKIIYDGSHVLGLIAGGQFQNPLQEGCDILIGNTHKTFPGPQKAMILYKDEELGRRIGSELFKSVVSSQHTHHALALYVTIIEMAAHGKLYAEQIVKNAEVFSRELITQGFNIVTRKGHLPVSHMVGIKGRFPQDNQFSAARLYMADISCNTKKIFGDNCIRIGVQELTRRGMKEEEMRCIARFFKRIIHNEDSSAALEVQQLNNRFNKVMYSLDTEYQQYLKR

SEQ ID NO: 29 - protein, 433 aa, Elusimicrobia bacterium
MNLAAAPPDPALAELRGLLGALKADEADYSEVVNLTANENTLSKTARSVLGSALGDRYFVGVWGDREASDDGGAYYVDEGLLVKGMPAAAGLERLAARLANSMFHSRYCDFRPLSGMCAVTSVIAAATQADDRFYIFAPKTLGHHASAALLTRMGRKVEFLPWEASSMSVDLEALRRKVRAAPPRAVLLDYGSPFYPLPTREIREIIGPEPLLVYDGSHVLGLIAGGQFQDPLNEGCDILIGNTHKTFPGPQKGLILYRDARLGKEVSDVINVTTVSTQQTHQSLALFIAMVEMGVHAADYAAQVLANSKAFSSALEAGGFDLLGLAGRPSETHMVAVQGPFSGDNHAACGALQDINLNANSKGILGRGVIRLGVQDATRRGMKEPQMRELAALMRERLLGGRPGTPLKARARELARAFGGLHYTLDEELSRP

SEQ ID NO: 30 - protein, 440 aa, Psuedomonas fluorescenes
MSNVKQQTAQIVDWLSSTLGKDHQYREDSLSLTANENYPSALVRLTSGSTAGAFYHCSFPFEVPAGEWHFPEPGHMNAIADQVRDLGKTLIGAQAFDWRPNGGSTAEQALMLAACKPGEGFVHFAHRDGGHFALESLAQKMGIEIFHLPVNPTSLLIDVAKLDEMVRRNPHIRIVILDQSFKLRWQPLAEIRSVLPDSCTLTYDMSHDGGLIMGGVFDSPLSCGADIVHGNTHKTIPGPQKGYIGFKSAQHPLLVDTSLWVCPHLQSNCHAEQLPPMWVAFKEMELFGRDYAAQIVSNAKTLARHLHELGLDVTGESFGFTQTHQVHFAVGDLQKALDLCVNSLHAGGIRSTNIEIPGKPGVHGIRLGVQAMTRRGMKEKDFEVVARFIADLYFKKTEPAKVAQQIKEFLQAFPLAPLAYSFDNYLDEELLAAVYQGAQR

SEQ ID NO: 31 - protein, 440 aa, Pseudomonas_sp._Irchel_s3a18
MKQDESNVGPVIDWLAQTLGQDYKYRQDTLSLTANENYPSELVRLTSGSTAGAFYHCSFPFPVPLGEWHFPEPGQMNEIADDLRGLAKRMMGAQAFDWRPNGGSPAEQALMLAACKQGEGFVHFAHRDGGHFALEQLATKMGIEIFHLPVDPQSLLIDVAKLDDMVRRNPHIRIVILDQSFKLRWQPLAEIRAILPDSCTLTYDMSHDGGLILGGVFDSPLACGADIAHGNTHKTIPGPQKGFIAFKSAQHPLLVETSLWVCPHLQSNCHAELLPSMWAAFKEMEAFGPAYAHQMVRNAKALANQLHELGLNVSGESFGFTETHQVHFAVGDLQQALSMCVDSLHAGGIRSTNIEIPGKPGMHGIRLGVQAMTRRGMKEDDFRRVAGLIADLYFKRTEPARVASKVKELLGDFPLAPLAYSFDQQIDESRRRLLERGIQR

SEQ ID NO: 32 - protein, 440 aa, Burkholderia stagnalis
MKQEPTGAFEVATVLNDIFLADHRYREVTLSLTANENYPSELVRVTSGSTAGAFYHVSFPFDVPDGEWHFPEPGHMHAVADKVRSLGKSLLHAQTFDWRPNGGSAAEQALMLAACQPGDGFVHFAHGDGGHFALEALASKAGIEIFHLPVDPDTLLIDVNRLATLVDAHPRIRIVILDQSFKLRWQPLRAIRDALPAHCTLTYDASHDGGLVMGGWFDSPLRCGADVVHGNTHKTIAGPQKAYVAFGSAEHPLLADTSIWVCPNIQSNCHAEQLPSIWVALKEIEAYGPAYASQVVRNATAFARALHARGLDVSGESFGFTETHQVHFSVGTPEAALLTCRDVLHRGGIRTTNIELPGKPGVHGIRLGVQAMTRRGMVERDFETVADFIAALCTRKRTPEDVAPDVETFLGDFPLSPLAFSFDGGMTDALRAALRQGVMR

SEQ ID NO: 33 - protein, 440 aa, Chitiniphilus shinanonensis
MTRTTPQARHVVERLNSVLGQDYRYREDCLSLTANENYPSALVRLAGSATAGAFYHCSFPFEVPPGEWYFPESGRMGELAQQLNELGRSLLGAGTFDWRPNGGSPAEQALMLAACKHGEGMVHFAHRDGGHFALENLAQKAGIDIFHLPVDPQTLLIDVARLDELVRRNPQIRIVILDQSFKLRWQPLAAIRKVLPPSCTLTYDTSHDGGLIMGGVFDSPLHCGADVIHGNTHKTVPGPQKGYIAFKSAEHPLLVDTSLWLCPHLQSNCHAELLPPMWVAFKEMEAFGHDYAPQVARNAKALAGHLHRLGFEVSGEAFGFTETHQVHFAVGDLQQALDLCMNTLHRGGIRSTNIEIPGKPGIQGIRLGVQAMTRRGLREDDFEQVARFIADLHFRKADPAGVAAQVAEFLRAFPLAPLHYSFDQELDHELLQSLIGEALR

SEQ ID NO: 34 - protein, 457 aa, Burkholderia ubonensis
MTDFAQAVVNPFVDEQRKSRLVEKISNIFDSLHSDFALDNLYRASHLSLTASENYPSRFVRTLGAGMQGGFYEFAPPYAANPGEWYFPDSGAQSSLVEKLASLGKQLFEANSFDWRPNGGSAAEQAVLLGTCARGDGFVHFAHKDGGHFALEELAQKVGVSIFHLPIEEKSLLIDVDRLATLIKDNPHIKLVILDQSFKLRWQPLLQIRQALPESVVLSYDASHDGGLIIGECLPQPLLFGADIVHGNTHKTIPGPQKGYIAFKNVDHPAMKHVSDWVCPHLQSNSHAELIAPMYIALVEMSLYGRSYAEQVIKNAKALAHALHAEGVRVSGESFGFTETHQVHVVVGSERKALELVTGTLALAGIRCNNIEIPGANGLFGLRLGVQALTRRGIKEHGMAEVARFLVRLILKNESPTAIRNEIASFLESYPINTLHYSLDAHYYTPSGIKLMEEVIA

SEQ ID NO: 35 - protein, 440 aa, Streptomyces (multi-species)
GVWAGDRVAQVLERLASDFVLDNTYREQHLSLTASENYPSKLVRMLGAGLQGGFYEFAPPYPAEAGEWAFPDSGANASLVGKLTGIGRQLFEAATFDWRPNGGSVAEQAVLLGTCGRGDGFVHFAHKDGGHFALESLAGAAGVNTYHLPMVDRTLLIDVDRLATLCAEHPEIKLVILDQSFKLRWQPLAQIRAALPEGVFLAYDASHDGALIAGGVLPQPTLLGADAVHGNTHKTIAGPQKAYIAFRDAEHPKLRAVSDWVCPQMQSNSHAELIAPMYVALSEVALYGHAYARQILANAQALAHGLHEEGVRVSGESFGFTETHQVHVVTGSAADALRLSLGELAQAGIRTTNIEVPGANGLHGLRLGVQAMTRRGLREPQMREVARLVAKVVLRRAEPAAVRAEVADLLQHHPLDQLAYSFDSYVDSPAAARLLGEVFR

SEQ ID NO: 36 - protein, 431 aa, Thermocladium modestius
MREEEAIAALSKLRAIMDRHNNWRRRETINLIPSENVMSPLAEYFYLNDMMGRYAEGTIGKRYYQGVSLVDEAEQMLVDLMSSLFSSRFTDVRPISGTVANMAVYHSVAGLGEKIASLPTAAGGHISHNETGAPKAFGLRVSYLPWSQENFNVDVDAARRLIAEERPKLVLLGASLYLFPHPIKELADAAHEVGAVLMHDSAHVLGLIAGHQFPNPLELGADIMTSSTHKTFPGPQGGVIFTTREDLFKEIQRSVFPVMTSNYHLHRYASTIVTAIEMSTYGDEYAATVRSNAKALAEQLHANGLPVVAEEHGFTATHQVAMDVSKFGGGGPIAKALEDANIIVNKNMLPWDKSPVKPSGIRMGVQEMTRMGMGKGEMAAVAELIAKVVIKGVEPSKVKPEVVELRRGFTKVRYGFDLSTLGLNCPCLPLL

SEQ ID NO: 37 - protein, 431 aa, Rugosimonospora africana
MLEIVGDHERKMASAVNLIPSENLLTPAARLAYLSDAYSRYFFDEREVFGKWSFQGGSIVGEVQREVLVPLVQKVTGARHVDVRGISGLNAMTVALAAFGARDRVTITVPPRHGGHPATAVVAGHFGHRAEALPFRDEAWWEVDLPALAELVARTDPALVYVDQATALVPLDLAGVIRTVKEVSPGTHVHADTSHINAFVWSGLFGQPLDLGADSYGGSTHKTFAGPHKALLLTNDDAVSDKLTSVAVNLVSHHHVSDVVALAIAMVEFAECGGVDYAQAVLANAAAFARALADAGPGVQDAGGVLTRTHQVWYEPAGDPHRISERLFDAGIVVNPYNPLPSTGRLGIRMGLNEATKLGFGEPEMAELAGLLHGVAVDRIAVAEAGERVAAMRQAARPAYCFSEDVVASKLRELTGASGAGVDELAAWLYR

SEQ ID NO: 38 - protein, 434 aa, Streptomyces sp. NRRL 30471
MTSSDDCAASRTAPVAGRAELLALLGEIEKEQRINEAAVNLVPSENRISPWAGAPLRTDFYNRYFFNDSLDPQGWQFRGGEGIGRLEKELALPALRRLGRADHVNIRPVSGMSAMLVVLLGLGGEPGDGVVCVDAETGGHYATGRQIAMLGRRPLPVRVVAGRVDLDALRTALTSCHVPLVYLDLQNSLWELDVAGVAEVIARTSPRTVLHVDCSHTLGLILGGSHKNPLDLGADTTGGSTHKTFPGPQKGVLFTRDENLSRKIRDAQFFTISSHHFAETLALALAAAEFEHFGAAYSRQVLINARAFAHRLRERGFGVVEGGPQLTDTHQVWVRLPLEESADAFSAQLASLGIRVNVQTELPDIPEPALRLGVSEITLNGGREPAMETLAEIFALVRAGEATKAVDLFQVLPHEMGEPYFFTGLPQEAGLFHG

SEQ ID NO: 39 - protein, 425 aa, Nocardia otitidiscaviarum
MNTFDILEQLARYEVGTSRRLHLIASENPLDSDTRVPYMLAGTLARYAFGEPGQPNWAWPGRETLIDLEADTAAALGALLGADHVNLRPTSGLSAMTVALSALAEHAGDRATVLSLAESDGGHGSTGFMARRFGLDWQRMPADPRTGVVDLDALARQARSARGPLVLYLDAFMARFPFDLTGIRGAVGDSALIHYDGSHPLGLIAGGRFQNPLAEGADSLGGSVHKTWPGPVGKGIIATNDSALASRFDTHAAGWISHHHPADLAALALSTAWMEQHAGDYATAVIANAVQLADELADGGLSICADDRGATASHQVWVDIAPICPAPVAAQRLYDAGIVVNAIAIPGLAEPGLRLGVQELTRWGLDRDGMTVLTWVLTQLLVHNAATAVVAPQMEALRTGLTLPEDRHGLEGFLRACDPQEVSVA

SEQ ID NO: 40 - protein, 382 aa, Deltaproteobacteria bacterium
LTNNRELMDRIGYNLSQGLVSSQHTASLVALFIALHEARLTGKAFAKQVVENARTLASRLAALGVPVLARSDGQFTDNHHFFINLTGVASAPHQMERLLRAHLVVQRGMPFRNVDALRVGVQEVTRRGYGPGEMAQLAEWIASIVIGGADPEVVAPAVQAMAKRFDTIYYTGETVDGKLDLPEIAAPSAKGRWVDYRHLGNDFAMDDTEFSEIRALGAAAGAFPNQTDSTGNVSLRSGARVFVSSSGSYIKHLADGQVVELDAVDPSGELIDYHGAALPSSESLMHFLVYQNVPAGAVVHTHYLLTNQEAADFDVAVIAPQEYASIALARAVAEASKRSRIVYIQKHGLVFWGTDTADCLSQVHNFIHNRPNRRAAEAVYAS

SEQ ID NO: 41 - protein, 100 aa, Saccharomyces cerevisiae
MSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGG

SEQ ID NO: 42 - DNA, 1238 bp, synthetic construct
  source 1..1238: mol_type = other DNA
ccatcaccatcatcaccacatggatgtgttggctgccctggaacgtaagcacagtttaaacttgtttccgattgaaaatcgcttgtcaccccgtgctgccgccgctctggcatccgatgccgtaaaccgttatccgtacagtgagacggatgtggcggtgtacggagacgttagtgatctgaatgctgtatatgaccattgcgtcagtcttaccaaggaattttatggcgcccgtcatgcatatgttcagtttctttccggacttcacaccatgcatacagtgttaacagcagtcacaccgccagggggccgtgtaatggtcattgcgcctgaagacggaggacattatgcaacggttactatttgccaaggttttggctaccgcgtagagtacgtaccattcgatcgccagactttggaaattgactacactgctcttgccgaacgcacagccgaacatccggctgatgtgatctacttggacgcatcgacggtattgcgcatgcctgacgcgcgcgctctgcgtgcagcagccccaggcgctgttctgtgtctggatgcaagtcatcttctgggacttcttcccgcagcccctgggaccttggtccttgatgctggctttgattcaatttctggaagcactcacaaaactttaccgggaccccaaaagggattgttggtgacaaactccgatgccattgccgaacaggtcggagcgcgcatcccttttaccgcgagttcatcgcattctgcgagcgtgggttcgctggcgattacattagaagagcttttgccccatcgcggggattacgcacgtcaggtgatcgcaaacgcccgtgagctggctcgtcaacttgcggcccgcggctttgacgtggcaggggaagccttcggatttactgatactcatcaggtgtgggtccaccatccagagggaaatacaccgcatgagtggggacgtctgctgacagctactgatattcgcaccactacagtagtgcttccatcaactgcacgtagtggattacgtttaggaacgcaggagttgacacgttgggggatgaaggaagacgatatgactaccgttgcagagcttcttgcccgtctgcttttacgcggagaacagagtcgctcagttgccgcggatgtacgcgacttggctcgttcgttcccaggtgtggctttcgcggaccgtccagcacccttggcagtagcctaacttgatgggggatcccatg

SEQ ID NO: 43 - DNA, 1313 bp, synthetic construct
  source 1..1313: mol_type = other DNA
ccatcaccatcatcaccacatggaaacctccctgaaggattttgaaactatccttcacttaattaataaggaggagattgactcaaatgacaccattcatatgaccgccaacgaaaatattatgtctaaattgtccaaacactacttaaaaagcactttgtcttaccgctaccatgtcggaatgttcgatgatcaaaagaacctgacagtctcgcgttcgtgtcttatcaaaaactctttgatgctgcgttgcctttcacccatcttcctgttagaacaacaagcccgtgaatacgtaaaaaaaatgttcttcgctgagtatgcggactttcgtcctttgtccggtatgcacaccgttttttgtatcttatctaccttaacaaaaccgaacgatcgtgtctatgtcttcacgaccgaatcggtaggacacgcagccacagtttctttattgaagtcgttgggtcgcaaagtgtccttcatcccattttgtgagaagaaacttgatattgacttagagaagctgagtaaacaaatcttgattgagaaacccaacgcaattctttttgattttggtactccattctacccattgccgatccgcgaaattcgcgagattgtaggaaacgacgtgaagatgatttatgacgcctcgcatgtgttgggtttgattgcgggtggacagttccaaaatccacttcttgaaggctgtgacgtgctgatcggaaatactcacaagacatttccggggccgcagaaaggcatgatcttgtataaaaacaagtctttgggaaaggagatcgcaacagaaattttcaaatcagccatttctgcgcagcatactcatcatgctatcgccctgtacgttactatcattgaaatgtatatccacgggaaggaatacgccaaccaaatcatcaaaaataatcatgcgttatcccaggcattaatcaatgaaggttttaaaatttttaagcgtaaaaaccagtttagccttagtcacatgattgcgattacgggggattttccgattgatcatcatgttgcatgtgccgatttgcataattctaacatctccacaaattcgcgtattctgtatgactttccagccgtgcgcattggcgttcaggaggttacacgtaaaggaatgaaagaaaaggatatggtgcaattagccaaattttttaaggaaatcatcctggatcgcaagaacatcagctctaaaatcaaggagttcaataacaaattcaatagtattgaatatagtcttgacgagatctacgagaagttattctaacttgatgggggatcccatg

SEQ ID NO: 44 - DNA, 1323 bp, synthetic construct
  source 1..1323: mol_type = other DNA
atgtccaatgtcaagcaacagacagctcagatcgtggattggttatcaagcactttaggtaaagaccatcagtatcgtgaagatagcttgagtcttacagcgaacgagaactatccgtcagcgttggtacgtttgacgtcgggctcgaccgcaggggcgttttatcactgtagtttcccctttgaggtacctgccggggaatggcacttcccggagccagggcatatgaatgccatcgcagaccaggtacgtgatcttgggaaaacactgatcggagcacaggcgtttgactggcgcccaaacggcggctctacagcagaacaggcgttgatgttagcggcgtgcaagcccggggaaggatttgtccatttcgcacaccgcgacggaggccattttgcgcttgaatcactggcgcagaaaatgggaattgaaattttccacttgccagttaaccccacgagtttgcttattgatgtggcgaaattggatgaaatggtccgccgcaatccgcacatccgtattgtaattctggaccagtcctttaagcttcgctggcagccgttggcggaaattcgttccgtactgccggattcgtgtactttgacgtacgacatgagtcacgatggaggtttgattatgggtggcgttttcgattcgcctttaagttgcggagcagacatcgtacacggaaacacacataagacgatccctggtccacagaaagggtacatcggatttaagagtgctcaacacccgctgttagtggataccagcctttgggtatgccctcacctgcaatccaactgccatgcggaacagctgccgccaatgtgggtagcattcaaagaaatggaactgttcgggcgtgattacgcggcccaaattgtgtcaaatgctaagaccttggcacgtcacttgcacgagttaggattagacgttacgggggagagctttgggtttacccagactcaccaggtacacttcgctgtaggcgacttacaaaaagccttggatttatgtgttaattcacttcacgcagggggcatccgtagcacgaatatcgagattcccggaaaaccaggggtgcatggtattcgtttgggtgtgcaagcgatgactcgccgtggcatgaaggaaaaggatttcgaggtggtagctcgtttcattgcggatctttacttcaagaaaactgagccagcgaaagttgctcagcagattaaggaatttttgcaggcgttcccattagcgcctctggcatattcttttgataattatttagacgaggagttattggctgcggtgtaccaaggagcccaacgttaa

SEQ ID NO: 45 - DNA, 1323 bp, synthetic construct
  source 1..1323: mol_type = other DNA
atgaaacaagacgaatcgaatgttggtcctgtcattgactggctggctcagacccttggacaggactacaagtaccgccaggacacactttcacttacagctaacgaaaactacccttcagagcttgttcgtctgaccagcggctctacagccggggcattttatcactgctcttttccgttccccgttcctcttggagaatggcatttcccagagccaggacaaatgaacgagatcgccgatgatctgcgcggtttggccaaacgtatgatgggtgcgcaggcattcgattggcgccctaatggtgggagcccggctgaacaggccttgatgttagcggcttgtaaacaaggtgaaggttttgtacactttgcacatcgcgatggggggcattttgctttagagcaattggcgacaaaaatgggtattgagattttccatttacctgtggatccgcaaagtctgcttattgacgttgctaagcttgatgacatggtccgccgtaaccctcacatccgtatcgtaattcttgatcaatccttcaaacttcgttggcagccgttagccgagattcgtgcaatccttcccgattcatgcactttaacttatgatatgtctcatgatgggggccttattctgggtggggtcttcgatagcccattggcgtgcggtgcggatatcgctcacggcaatactcacaagactattccggggcctcaaaaggggtttattgcgttcaagagcgctcagcaccccctgttggtggaaaccagtctttgggtatgtccacacttacagagtaactgtcacgccgaacttttaccctctatgtgggccgcattcaaggagatggaagcttttggccccgcctatgcccaccagatggtgcgcaatgctaaggcgttggccaaccaacttcacgagcttggtttaaatgtttcgggagagtcttttggatttacagagacgcaccaggtgcatttcgccgtaggagatttacaacaggcgttgagtatgtgcgtggactcgttacacgcgggcggaatccgctcgactaacatcgagatcccgggaaagcccgggatgcacgggatccgcttgggggtacaggccatgacccgccgcggtatgaaagaggatgactttcgtcgcgtcgctggccttatcgctgacctttacttcaagcgtaccgaacctgcacgtgttgcttcaaaggtgaaggagttattgggcgattttccacttgcccctctggcctactcgttcgatcaacaaatcgacgagtctcgccgccgtttgcttgagcgtggaattcaacgctaa

SEQ ID NO: 46 - DNA, 1323 bp, synthetic construct
  source 1..1323: mol_type = other DNA
atgaaacaggaacctacgggcgccttcgaggttgccacggtgctgaacgacatttttcttgctgaccatcgctaccgcgaggtaactcttagtcttaccgctaatgaaaattatccttcagagcttgtacgtgttacgtccggaagtaccgccggggctttttatcatgtgagcttcccgttcgatgtacccgatggagaatggcacttccccgaacccggacatatgcacgcggtggcggataaagttcgtagtttggggaagtcattgctgcatgcacagacatttgattggcgtccaaacggtggctctgcggcggaacaggcgttaatgcttgcggcctgtcaacccggtgatggtttcgttcatttcgcacatggagacggagggcacttcgccttagaggctctggcatcaaaagcaggtatcgaaatctttcatctgccagttgacccagacacgctgcttattgatgtgaatcgtttagctacgttagtggacgcacatccacgtattcgtattgtcattttggaccagtcatttaaacttcgctggcagcctctgcgcgcgatccgtgatgcacttcctgcacattgtacgttgacttacgatgctagccacgatggagggctggttatgggaggatggtttgacagcccgcttcgttgtggtgctgacgtagttcatggtaatacccataaaactattgcagggcctcagaaagcttatgttgcttttggctctgctgagcaccccttattagcagataccagtatttgggtgtgcccgaacattcagagcaattgtcatgcagaacagctgccatctatttgggttgcattgaaagaaatcgaagcatacgggcctgcatatgcgtcccaggtagtgcgtaacgcgacagcgtttgctcgtgctttacacgcgcgtgggcttgacgtgtcaggagagtcctttgggttcaccgaaacccatcaagtccacttcagcgtcgggaccccggaggcagcgttattgacatgtcgtgacgtgttgcaccgcgggggaatccgtaccacgaacatcgagcttccgggtaagccgggggtacatggcatccgtcttggagtacaggcaatgacgcgtcgtggaatggtcgagcgcgactttgaaaccgtcgccgactttatcgctgcgctttgtacacgcaaacgtacacccgaggatgtggctccggatgtcgaaacgttcctgggtgacttcccattatccccacttgcattttccttcgacgggggtatgactgacgcattgcgtgccgcactgcgccaaggcgtgatgcgttaa

SEQ ID NO: 47 - DNA, 1323 bp, synthetic construct
  source 1..1323: mol_type = other DNA
atgacgcgcacgaccccccaggcacgtcatgtcgtggagcgcctgaattcagttttaggacaagactaccgctatcgtgaggattgtctgagccttaccgcgaatgagaactatccttccgcattagtgcgcttagcggggagtgccacagctggagccttctaccactgtagctttccgtttgaggtgccaccgggagaatggtattttcctgagagcggtcgtatgggggaacttgctcaacagctgaatgaattaggtcgttcgttattaggcgcgggtacattcgattggcgccccaacggtggctcgccagcggagcaggcattgatgttagcggcctgcaagcacggtgaagggatggtccattttgctcatcgtgacggtggccactttgcgctggagaatctggcgcaaaaagctggtatcgacatctttcatttgcctgtagatccccagacgttgttgatcgatgttgcacgccttgacgagcttgtccgccgcaatcctcaaatccgtattgtgatcttggaccagtcttttaagttacgctggcaaccccttgcagcgatccgcaaggttcttcccccatcgtgtacacttacctatgacacctctcatgatggtggacttattatgggaggagtttttgattctcccttgcattgtggtgcagacgtaattcatggcaacacgcataaaacagtgcccggaccgcagaaggggtatatcgccttcaaatccgctgagcatcctttgttggttgacacgagtctgtggctttgcccacatttgcagtctaactgtcatgccgagcttttgcctccaatgtgggtggcttttaaagaaatggaggctttcggacatgattacgcccctcaagtggcccgcaacgcgaaggctctggcgggtcatttacatcgtttaggattcgaggtttcaggcgaggctttcggtttcactgaaacccaccaagtgcattttgccgtaggagacttgcagcaagcgcttgatttgtgcatgaacaccttgcatcgtgggggcatccgctctacgaatattgaaatcccgggtaaacccggcattcagggtattcgcctgggcgttcaggctatgacccgtcgcggtctgcgcgaagatgattttgagcaggtggcgcgttttatcgcggacttgcacttccgcaaagcagacccagccggagtcgcagcacaagtagcggaatttcttcgtgcttttcctttggcaccattacattactcatttgatcaggaactggatcatgagttattgcagtcccttattggggaggcgttacgctaa

SEQ ID NO: 48 - DNA, 1377 bp, synthetic construct
  source 1..1377: mol_type = other DNA
atgatgacggacttcgcacaggcggtagtaaacccgttcgtagatgagcagcgtaagtcccgtttagtagaaaaaatctcaaacatcttcgatagtcttcatagcgattttgccttggataatttataccgcgcaagccacttaagtctgaccgcctctgagaattatccatcccgctttgtgcgcacgctgggagccggtatgcaaggcggtttctatgaattcgcgccaccttacgccgctaacccaggagagtggtacttccctgacagtggcgcgcagtcgagtctggtcgagaaacttgctagtttgggaaaacagttgttcgaggctaactcgtttgactggcgtcccaacgggggatcagcagcggaacaggctgtgcttttaggcacatgtgcccgcggggatggcttcgtccactttgctcacaaggatggcggccactttgctctggaagagttggcccagaaggtgggagttagcatcttccatctgccaatcgaggagaagagtcttttgattgatgttgaccgcctggcgacattaatcaaagataacccccacattaagcttgtaattctggaccaatcgtttaagcttcgctggcaacctttactgcaaatccgccaagccttaccggaatcagtcgtattatcgtacgacgcgagtcacgacgggggattaatcatcggcgaatgcctgccccagccattacttttcggagcggatattgttcacgggaatacacacaagacaattccgggcccgcaaaagggttacattgcgttcaagaatgtagaccatcctgcgatgaagcatgttagcgattgggtttgtcctcatttgcaatctaactcgcatgccgagttgatcgcacccatgtatattgccttggttgaaatgtctttgtacggacgcagttacgcggagcaggttattaaaaatgctaaggcgttggcacacgccctgcacgccgagggagtacgcgtctcgggcgaatcgttcggttttacagaaacacaccaagttcatgttgttgttgggtccgagcgtaaagcgttggagttagttactggtaccttggcattggcaggaattcgctgcaacaacatcgagattccaggcgcgaacggcttatttggtttgcgcttaggagtgcaggcattgacgcgtcgcggaattaaagagcacgggatggctgaagttgcccgttttttagtgcgcttgattctgaaaaacgaatcccccacggccatccgcaacgaaattgcgtcatttcttgaatcatatcctattaatacgcttcattattcattagatgctcactattataccccttcgggtattaaattgatggaggaagtaatcgcttaa

SEQ ID NO: 49 - DNA, 1326 bp, synthetic construct
  source 1..1326: mol_type = other DNA
atgggagtttgggcaggcgaccgtgttgcccaagttttggaacgcttagcgtcggattttgttttagacaacacttatcgcgaacaacacctgagcttgacggcttctgagaactatccttcaaaactggtacgcatgttgggagcgggattacaggggggtttctatgagtttgctccgccctatccggcagaagcaggagaatgggcattcccggactccggagcgaacgcgtcccttgtagggaagctgactggcattggtcgccaactgttcgaagccgcaacattcgactggcgtccgaacggcggatccgtggccgagcaagcagtattgctggggacgtgtggacgcggggatggttttgtgcacttcgcgcataaggatgggggccactttgcgttggagagtctggcgggtgctgccggagtcaacacgtatcatctgcccatggtagaccgcacgcttctgatcgatgtcgatcgtttggctactttatgcgctgaacacccggaaattaagttagtaatcttagatcagtccttcaaattacgctggcaaccgcttgctcaaatccgcgccgcgctgcccgagggcgtatttttagcttatgacgcgtctcatgacggtgctttgattgctgggggtgttctgccacagcctaccctgttaggggccgatgcagttcatggcaacacgcacaaaacgatcgcggggcctcaaaaggcgtatattgcgttccgcgacgctgagcaccccaagttacgtgccgtcagtgattgggtgtgtccacagatgcagagtaattcacatgcggaactgatcgcacccatgtatgtagcactgtcggaggtcgccttatatggtcatgcgtatgcccgccaaatcttagcaaacgcccaagcgttagcgcacggattacacgaagagggggtccgcgtatctggagagtccttcggctttacagaaactcatcaagtacacgtcgtgacgggttcagctgcggatgctctgcgcctgtccttgggtgagctggcccaggcaggaatccgtacgacaaacattgaggtaccaggggcaaatggactgcatggtttgcgcttaggagttcaagctatgactcgccgtggtttacgcgagccacagatgcgtgaagtggcacgcttggttgccaaagttgttttgcgccgtgccgaaccagcggctgtacgcgcggaggttgcggatttgttacagcatcacccgttagatcagttggcgtattccttcgattcctacgttgactcgccagctgcggcgcgtttgttgggtgaggtctttcgttaa

SEQ ID NO: 50 - DNA, 1334 bp, synthetic construct
  source 1..1334: mol_type = other DNA
ccatcaccatcatcaccacatgcgcgaggaagaagcgattgcggcgctgtcaaaattacgcgcaatcatggaccgccataacaactggcgccgccgtgagacaattaacttaattccaagcgaaaacgtgatgtcgccgttagccgagtatttctacttaaatgatatgatgggacgttatgctgaaggaacgattggtaaacgctactaccaaggtgtatcgctggtggacgaggcggaacaaatgttagtcgatttaatgagctctttgttttcctcgcgctttacagacgtccgccccatcagcggtacagttgccaatatggccgtgtatcactcagtcgcggggcttggggagaagatcgcctctttaccaacagccgccgggggccatatttcgcataacgagactggtgcccccaaagcattcggattacgtgtttcatatttgccgtggtctcaggaaaactttaacgtggatgtggacgctgcgcgtcgcttaattgccgaagaacgcccaaaattggtgttgcttggggcgtcactttatttatttcctcatcccattaaagaattagcggacgctgctcacgaggtaggtgcggttctgatgcatgactcagctcacgtacttggtttaattgctggtcatcagttccctaatcctcttgaacttggggcggacattatgactagcagcacgcacaaaacttttccgggaccccaaggcggtgtgatttttaccacacgtgaagatttgttcaaggagatccaacgctcagttttcccagtaatgacatcgaattatcacttgcatcgctatgcctcgacgattgtgacagctattgagatgagtacgtatggagacgaatatgcagctacagtgcgctccaacgcgaaagcactggcggaacaacttcatgccaacggtttacctgtagttgccgaagaacacggcttcacggctacccaccaggtggcaatggatgtttcaaaatttggaggcggggggccaatcgctaaagcgttggaggacgcgaatattattgtaaacaagaacatgctgccctgggataagtctccggtcaaaccatccggtattcgcatgggagttcaagaaatgactcgcatgggaatgggtaaaggcgagatggcggccgtggcggagctgatcgcaaaggtggtcatcaaaggggtcgaaccgtctaaagtaaagccagaggtcgtcgagttgcgccgcggtttcacaaaggtacgctatggttttgatttatctactttgggcttgaattgcccttgtcttccgttactgtaacttgatgggggatcccatg

SEQ ID NO: 51 - DNA, 1334 bp, synthetic construct
  source 1..1334: mol_type = other DNA
ccatcaccatcatcaccacatgttggaaattgtgggggaccatgaacgcaaaatggcgagtgcagtgaatcttatccccagcgagaatttattaacacccgccgcacgtttagcctacctttcagatgcgtattcgcgttattttttcgatgagcgtgaggtgttcggaaagtggtctttccaaggggggagcattgtgggcgaagtacaacgtgaggttttagtgcctctggtacaaaaggtaactggggcacgccatgtggacgtccgtgggattagtggcctgaatgccatgaccgtggctctggcagcgtttggcgcccgtgaccgcgttacaattacagtaccgccccgccacggaggccatccagctaccgcagttgtggccggacactttgggcatcgtgcagaggctttacctttccgtgatgaagcctggtgggaggttgacttgcctgccttagcggagttagtagctcgtactgatccggcgttagtttatgtagatcaggccaccgctctggtcccactggatttagccggagtaatccgcaccgtcaaggaagtttcccctgggacacacgtacacgccgacacatcgcacatcaacgcgttcgtttggtcgggattgttcggccaaccacttgacttgggggcggacagttacggaggctccacgcataagacctttgcgggccctcataaggctttattgcttactaacgatgacgcagtgagcgataaactgacctccgtcgcagtgaatcttgtttcgcatcatcatgtcagcgacgttgtagctttagctatcgccatggtagagttcgcggaatgtggcggggtagattacgcgcaggcagttttagcaaatgcagcggcgttcgcccgcgccctggccgatgccgggcctggcgtacaagacgcgggtggtgtcttaacccgtacgcatcaagtatggtacgaacctgctggcgatccgcaccgcattagcgagcgcttgttcgatgcggggatcgttgtgaacccttacaaccctctgccgagtaccggtcgtttaggaatccgtatggggttaaatgaggcgaccaagttaggattcggagaaccggaaatggccgagttagcagggttgcttcacggtgtagcggttgaccgtatcgccgtggctgaggcgggagagcgtgtggctgccatgcgtcaagccgctcgtcccgcgtattgtttttctgaagatgtggtcgcctctaagcttcgcgagcttaccggagcctcaggtgcaggtgtggatgagttggctgcgtggctttatcgttaacttgatgggggatcccatg

SEQ ID NO: 52 - DNA, 1343 bp, synthetic construct
  source 1..1343: mol_type = other DNA
ccatcaccatcatcaccacatgacatcaagcgacgattgtgctgcgagtcgtacggctcccgtcgctggccgcgcagaacttttggcgctgttgggagaaatcgagaaggagcagcgcatcaacgaggccgccgtgaacttagtgccttcagagaatcgcattagtccctgggctggggcgccgttacgtaccgatttttacaaccgctatttcttcaacgattctctggacccccagggatggcaatttcgtggaggggaagggattggacgcctggaaaaggagttggctctgcccgctttacgccgtttagggcgtgccgatcacgttaacatccgtcctgtgtcaggtatgagtgccatgcttgtggtccttttaggtttgggaggcgaacctggggatggtgtagtgtgtgtagacgcagaaacgggaggtcattatgctactggccgccaaatcgcaatgttaggccgccgccctttgcccgtccgcgtggtagcgggacgcgttgatttggatgctcttcgcacggcattaactagctgccacgttcccttggtatatcttgaccttcagaattcactttgggagcttgatgttgcgggagtagccgaggtcatcgcacgtacaagcccacgtactgttctgcacgtggactgcagccacacattaggattaatccttgggggctcacataaaaatccattagacttgggtgcggatacgactggggggtcgacccataaaactttcccaggtccgcagaaaggggttttgttcacacgtgacgagaacttgagtcgtaagatccgtgatgctcaatttttcacgatcagttcacatcacttcgcggaaacactggcgttggccttagcggctgcagaatttgagcattttggcgcagcctatagccgccaagtccttatcaatgctcgcgcttttgcacaccgcttacgcgagcgcggatttggagtcgttgaaggcggcccgcagctgacggatactcaccaagtctgggtccgcttacctcttgaagaatcggcagatgcctttagcgctcaattggcgtccttaggtatccgcgtcaatgtccagactgagttgccagacatccctgaaccagccctgcgcttaggcgtgagcgagattactcttaatggtggacgtgagccagcaatggaaacgttggcagagatcttcgctttggtacgcgcaggggaggcgactaaggctgtcgatttattccaagttcttccccatgaaatgggggaaccgtatttttttacgggattacctcaagaagcgggactttttcatgggtaacttgatgggggatcccatg

SEQ ID NO: 53 - DNA, 1316 bp, synthetic construct
  source 1..1316: mol_type = other DNA
ccatcaccatcatcaccacatgaatacgttcgatatcttagaacaacttgcacgttatgaggtaggcacatcgcgccgtttgcatttaattgcgtctgagaatcccctggactcagacacacgtgtgccgtatatgcttgcaggaactttagctcgttacgcatttggggagccgggtcagcccaactgggcttggccaggccgtgagactctgattgacctggaagctgacactgcggcagcccttggggctttgctgggcgccgatcatgttaatcttcgtccgactagtggtctttcagctatgaccgtggccttgtccgccttggccgaacatgctggggaccgtgcaactgttttatcgcttgcagaatcagatggtggccatggatcgacggggttcatggcccgtcgttttgggctggactggcaacgcatgcccgctgacccgcgtacaggcgttgtggatctggacgcactggcgcgtcaggctcgcagtgcccgcggtcctctggtcttatatctggatgcgttcatggcgcgctttccttttgacttaacgggtatccgcggtgcggtgggtgactcagctttgatccattacgacggttcacatcctttgggattaatcgcgggaggccgtttccaaaatccgttagctgaaggcgccgattcgcttggagggtctgtacacaaaacctggcctggaccggtagggaaagggatcatcgctaccaatgatagtgcacttgcatctcgcttcgatactcacgccgcgggttggatctcccaccatcaccctgcggatctggctgcactggcgcttagtaccgcctggatggagcaacatgctggcgactacgcgacagcagtgatcgcaaatgccgtgcaattagctgatgaacttgcagacggcggcttgagcatctgtgccgatgaccgtggtgctacggcgagtcatcaagtgtgggttgatattgctcctatctgtccagctcctgtcgcggctcagcgtttgtatgatgctggtattgtggtaaacgcgattgcaatcccagggcttgccgaacccggcttgcgcctgggcgttcaggagttgactcgctggggattagaccgtgatggaatgacagtcctgacctgggtactgacccaactgctggtccataacgcggccacagcagtggtggccccgcaaatggaagcgttgcgtaccggcctgacgctgcctgaagatcgtcatgggctggagggttttcttcgtgcgtgtgatccacaggaggtatcagtcgcataacttgatgggggatcccatg

SEQ ID NO: 54 - DNA, 1187 bp, synthetic construct
  source 1..1187: mol_type = other DNA
ccatcaccatcatcaccacttgacgaataatcgcgagcttatggaccgtatcggttataatctttcacaaggtttagtttcaagccagcataccgcaagtctggtcgctttatttattgcattacatgaagcacgcctgaccggcaaagcgttcgcaaagcaagtggtagaaaacgcccgtacgttggcgagtcgtttggcggcacttggcgttccggtgttagcgcgttcagatggccagtttaccgacaatcatcatttcttcatcaatttgaccggcgtggcgagtgctcctcaccaaatggagcgcttacttcgtgcccatttggttgttcagcgcggcatgccgtttcgcaacgttgacgccttgcgtgttggcgtgcaagaagtcacacgccgcggttatggacccggcgagatggcgcagctggcagagtggattgcgtcaatcgtcatcggcggtgcggaccccgaggtagtagcacctgccgtgcaagccatggctaagcgctttgacactatctattatacgggcgaaacggtggacggtaaacttgatcttccagaaatcgcagcgccgagcgctaagggccgttgggttgactatcgccatttgggaaatgattttgcaatggacgatactgagttctccgaaattcgcgccttgggtgctgccgcgggagccttcccaaaccagaccgacagtacaggtaacgtctcgttacgttcaggagcccgtgtattcgtgtcgtctagcgggtcatatattaagcacctggccgacggacaggtcgtcgagttggacgcggtagatccctcaggggaattgattgactatcatggtgcggcgttgcccagcagtgagagtctgatgcacttcttagtttaccagaatgtgccagcgggcgcagttgtgcacactcactatttattaaccaaccaagaggctgccgacttcgatgtggcggtgatcgctcctcaggaatatgccagtattgcacttgcccgcgcagtagcagaagccagtaaacgctcccgtatcgtgtatattcaaaaacacggattagtgttttggggtacagacactgcagattgtctgtctcaggttcacaactttattcacaaccgtccaaatcgtcgcgcagctgaggcggtctatgcctcttaacttgatgggggatcccatg

SEQ ID NO: 55 - DNA, 300 bp, synthetic construct
  source 1..300: mol_type = other DNA
atgtccctgcaggactcggaggttaaccaggaagcaaagccggaagtcaaaccggaagtgaaacccgaaactcacatcaatctgaaggtaagtgatggttcttcagagatattctttaaaattaaaaaaaccacgcctctgcggcgtcttatggaagcgttcgccaaacgacaagggaaagagatggatagcttacgttttctctatgatggcattcgcatccaggcggatcaagctccagaggacttggatatggaagataacgacattatcgaagcccatcgcgaacagattggtggc

SEQ ID NO: 56 - DNA, 22 bp, synthetic construct
  source 1..22: mol_type = other DNA
cttgatgggggatcccatggta

SEQ ID NO: 57 - DNA, 58 bp, synthetic construct
  source 1..58: mol_type = other DNA
gtggtgatgatggtgatggctgctgcccatggtacctttctcctctttaatgaattcg

SEQ ID NO: 58 - DNA, 43 bp, synthetic construct
  source 1..43: mol_type = other DNA
ccatgggatcccccatcaagttaacgaaagacctcacccaaca

SEQ ID NO: 59 - DNA, 44 bp, synthetic construct
  source 1..44: mol_type = other DNA
ccatgggatcccccatcaagttaagcgattacttcctccatcaa

SEQ ID NO: 60 - DNA, 41 bp, synthetic construct
  source 1..41: mol_type = other DNA
ccatgggatcccccatcaagttagcgttgaattccacgctc

SEQ ID NO: 61 - DNA, 39 bp, synthetic construct
  source 1..39: mol_type = other DNA
ccatgggatcccccatcaagttaacgttgggctccttgg

SEQ ID NO: 62 - DNA, 39 bp, synthetic construct
  source 1..39: mol_type = other DNA
ccatgggatcccccatcaagttaacgcatcacgccttgg

SEQ ID NO: 63 - DNA, 42 bp, synthetic construct
  source 1..42: mol_type = other DNA
ccatgggatcccccatcaagttagcgtaacgcctccccaata

SEQ ID NO: 64 - DNA, 38 bp, synthetic construct
  source 1..38: mol_type = other DNA
gccatcaccatcatcaccacatgggagtttgggcaggc

SEQ ID NO: 65 - DNA, 38 bp, synthetic construct
  source 1..38: mol_type = other DNA
gccatcaccatcatcaccacatgatgacggacttcgca

SEQ ID NO: 66 - DNA, 42 bp, synthetic construct
  source 1..42: mol_type = other DNA
gccatcaccatcatcaccacatgaaacaagacgaatcgaatg

SEQ ID NO: 67 - DNA, 40 bp, synthetic construct
  source 1..40: mol_type = other DNA
gccatcaccatcatcaccacatgtccaatgtcaagcaaca

SEQ ID NO: 68 - DNA, 40 bp, synthetic construct
  source 1..40: mol_type = other DNA
gccatcaccatcatcaccacatgaaacaggaacctacggg

SEQ ID NO: 69 - DNA, 36 bp, synthetic construct
  source 1..36: mol_type = other DNA
gccatcaccatcatcaccacatgacgcgcacgaccc

SEQ ID NO: 70 - DNA, 18 bp, synthetic construct
  source 1..18: mol_type = other DNA
gtgcccgaacattcagag

SEQ ID NO: 71 - DNA, 17 bp, synthetic construct
  source 1..17: mol_type = other DNA
gcgtatattgcgttccg

SEQ ID NO: 72 - DNA, 17 bp, synthetic construct
  source 1..17: mol_type = other DNA
accatcctgcgatgaag

SEQ ID NO: 73 - DNA, 20 bp, synthetic construct
  source 1..20: mol_type = other DNA
aaaggggtttattgcgttca

SEQ ID NO: 74 - DNA, 18 bp, synthetic construct
  source 1..18: mol_type = other DNA
gcgggtcatttacatcgt

SEQ ID NO: 75 - DNA, 43 bp, synthetic construct
  source 1..43: mol_type = other DNA
gaaaatctgtattttcagggcaaacaagacgaatcgaatgttg

SEQ ID NO: 76 - DNA, 23 bp, synthetic construct
  source 1..23: mol_type = other DNA
gccctgaaaatacagattttctg

SEQ ID NO: 77 - DNA, 39 bp, synthetic construct
  source 1..39: mol_type = other DNA
gaaaatctgtattttcagggcaaacaggaacctacgggc

SEQ ID NO: 78 - DNA, 38 bp, synthetic construct
  source 1..38: mol_type = other DNA
aaaatctgtattttcagggcggagtttgggcaggcgac

SEQ ID NO: 79 - DNA, 25 bp, synthetic construct
  source 1..25: mol_type = other DNA
cctggtatctttatagtcctgtcgg

SEQ ID NO: 80 - DNA, 38 bp, synthetic construct
  source 1..38: mol_type = other DNA
aaaatctgtattttcagggcacgcgcacgaccccccag

SEQ ID NO: 81 - DNA, 33 bp, synthetic construct
  source 1..33: mol_type = other DNA
gggaaacgcctggtatctttatagtcctgtcgg

SEQ ID NO: 82 - DNA, 40 bp, synthetic construct
  source 1..40: mol_type = other DNA
aaaatctgtattttcagggctccaatgtcaagcaacagac

SEQ ID NO: 83 - DNA, 42 bp, synthetic construct
  source 1..42: mol_type = other DNA
aaaatctgtattttcagggcgaaacctccctgaaggattttg

SEQ ID NO: 84 - DNA, 37 bp, synthetic construct
  source 1..37: mol_type = other DNA
aaaatctgtattttcagggcacggacttcgcacaggc

SEQ ID NO: 85 - DNA, 26 bp, synthetic construct
  source 1..26: mol_type = other DNA
acgcctggtatctttatagtcctgtc

SEQ ID NO: 86 - DNA, 39 bp, synthetic construct
  source 1..39: mol_type = other DNA
gccatcaccatcatcaccacatgttggaaattgtggggg

SEQ ID NO: 87 - DNA, 40 bp, synthetic construct
  source 1..40: mol_type = other DNA
ccatgggatcccccatcaagttaacgataaagccacgcag

SEQ ID NO: 88 - DNA, 19 bp, synthetic construct
  source 1..19: mol_type = other DNA
cttgatgggggatcccatg

SEQ ID NO: 89 - DNA, 19 bp, synthetic construct
  source 1..19: mol_type = other DNA
gtggtgatgatggtgatgg

SEQ ID NO: 90 - DNA, 37 bp, synthetic construct
  source 1..37: mol_type = other DNA
gccatcaccatcatcaccacatgcgcgaggaagaagc

SEQ ID NO: 91 - DNA, 42 bp, synthetic construct
  source 1..42: mol_type = other DNA
ccatgggatcccccatcaagttacagtaacggaagacaaggg

SEQ ID NO: 92 - DNA, 40 bp, synthetic construct
  source 1..40: mol_type = other DNA
gccatcaccatcatcaccacatgacatcaagcgacgattg

SEQ ID NO: 93 - DNA, 40 bp, synthetic construct
  source 1..40: mol_type = other DNA
ccatgggatcccccatcaagttacccatgaaaaagtcccg

SEQ ID NO: 94 - DNA, 45 bp, synthetic construct
  source 1..45: mol_type = other DNA
gccatcaccatcatcaccacatgaatacgttcgatatcttagaac

SEQ ID NO: 95 - DNA, 39 bp, synthetic construct
  source 1..39: mol_type = other DNA
ccatgggatcccccatcaagttatgcgactgatacctcc

SEQ ID NO: 96 - DNA, 39 bp, synthetic construct
  source 1..39: mol_type = other DNA
gccatcaccatcatcaccacatggaaacctccctgaagg

SEQ ID NO: 97 - DNA, 45 bp, synthetic construct
  source 1..45: mol_type = other DNA
ccatgggatcccccatcaagttagaataacttctcgtagatctcg

SEQ ID NO: 98 - DNA, 39 bp, synthetic construct
  source 1..39: mol_type = other DNA
gccatcaccatcatcaccacttgacgaataatcgcgagc

SEQ ID NO: 99 - DNA, 39 bp, synthetic construct
  source 1..39: mol_type = other DNA
ccatgggatcccccatcaagttaagaggcatagaccgcc

SEQ ID NO: 100 - DNA, 37 bp, synthetic construct
  source 1..37: mol_type = other DNA
gccatcaccatcatcaccacatggatgtgttggctgc

SEQ ID NO: 101 - DNA, 38 bp, synthetic construct
  source 1..38: mol_type = other DNA
ccatgggatcccccatcaagttaggctactgccaaggg

SEQ ID NO: 102 - DNA, 17 bp, synthetic construct
  source 1..17: mol_type = other DNA
atgtccctgcaggactc

SEQ ID NO: 103 - DNA, 68 bp, synthetic construct
  source 1..68: mol_type = other DNA
gccctgaaaatacagattttctgaacctccacctcccgacccaccaccgccgccaccaatctgttcgc

SEQ ID NO: 104 - DNA, 39 bp, synthetic construct
  source 1..39: mol_type = other DNA
tccgagtcctgcagggacatgtggtgatgatggtgatgg

SEQ ID NO: 105 - DNA, 37 bp, synthetic construct
  source 1..37: mol_type = other DNA
aaaatctgtattttcagggcatgcgcgaggaagaagc

SEQ ID NO: 106 - DNA, 39 bp, synthetic construct
  source 1..39: mol_type = other DNA
aaaatctgtattttcagggcatgttggaaattgtggggg

SEQ ID NO: 107 - DNA, 40 bp, synthetic construct
  source 1..40: mol_type = other DNA
aaaatctgtattttcagggcatgacatcaagcgacgattg

SEQ ID NO: 108 - DNA, 45 bp, synthetic construct
  source 1..45: mol_type = other DNA
aaaatctgtattttcagggcatgaatacgttcgatatcttagaac

SEQ ID NO: 109 - DNA, 40 bp, synthetic construct
  source 1..40: mol_type = other DNA
tccgagtcctgcagggacatgtggtgatgatggtgatggc

SEQ ID NO: 110 - DNA, 39 bp, synthetic construct
  source 1..39: mol_type = other DNA
aaaatctgtattttcagggcttgacgaataatcgcgagc

SEQ ID NO: 111 - DNA, 37 bp, synthetic construct
  source 1..37: mol_type = other DNA
aaaatctgtattttcagggcatggatgtgttggctgc

SEQ ID NO: 112 - DNA, 17 bp, synthetic construct
  source 1..17: mol_type = other DNA
aagcttgatgggggatc

SEQ ID NO: 113 - DNA, 26 bp, synthetic construct
  source 1..26: mol_type = other DNA
ggtatatctccttattaaagttaaac

SEQ ID NO: 114 - DNA, 37 bp, synthetic construct
  source 1..37: mol_type = other DNA
ctttaataaggagatataccatgggcagcagccatca

SEQ ID NO: 115 - DNA, 44 bp, synthetic construct
  source 1..44: mol_type = other DNA
ggatcccccatcaagcttttagaataacttctcgtagatctcgt

(19) United States
(12) Patent Application Publication
(10) Pub. No.: US 2025/0215467 A1
(43) Pub. Date: Jul. 3, 2025

(54) L-THREONINE TRANSALDOLASES AND USES THEREOF

(71) Applicants: Aditya Kunjapur, Newark, DE (US); Michaela Jones, Eikton, MD (US); Neil Butler, Newark, DE (US); Sean Wirt, Cambridge, MA (US)

(72) Inventors: Aditya Kunjapur, Newark, DE (US); Michaela Jones, Eikton, MD (US); Neil Butler, Newark, DE (US); Sean Wirt, Cambridge, MA (US)

(73) Assignee: University of Delaware, Newark, DE (US)

(21) Appl. No.: 18/847,362

(22) PCT Filed: Mar. 17, 2023
(86) PCT No.: PCT/US2023/064643

§ 371 Date: Sep. 16, 2024

Related U.S. Application Data
(60) Provisional application No. 63/320,859, filed on Mar. 17, 2022.

(51) Int. Cl. C12P 13/04; C12N 9/02; C12N 9/10; C12P 7/00

(52) U.S. Cl. CPC C12P 13/04; C12N 9/0008; C12N 9/1022; C12P 7/00; C12Y 102/0103; C12Y 202/01002

(57) ABSTRACT

The invention provides a method for producing in vitro a beta-hydroxy non-standard amino acid (0-OH-nsAA). The in vitro method comprises incubating L-threonine, an aldehyde and an L-threonine transaldolase (TTA). Also provided is a method for producing a beta-hydroxy non-standard amino acid (0-OH-nsAA) by recombinant cells, comprising expressing a heterologous L-threonine transaldolase (TTA) by the recombinant cells, and growing the recombinant cells in a medium. The medium comprises L-threonine and an aldehyde.

### CROSS-REFERENCE TO RELATED APPLICATION

This application claims priority to U.S. Provisional Application No. 63/320,859, filed Mar. 17, 2022, and the contents of which are incorporated herein by reference in their entireties for all purposes.

### REFERENCE TO U.S. GOVERNMENT SUPPORT

This invention was made with government support under Grant No. MCB2027092/CBET2032243 from the National Science Foundation, Award No. N000142212536 by the Office of Naval Research, Grant number P200A210065 by the Department of Education—Graduate Assistance in Areas of National Need, Chemistry-Biology Interface Training Grant No. T32GM133395 by the National Institute of General Medical Sciences of the National Institutes of Health, Collaborative Research Grant No. MCB-2027074 by the National Science Foundation, and Award Number P20GM104316 by the National Institute of General Medical Sciences of the National Institutes of Health. The United States has certain rights in the invention.

### FIELD OF THE INVENTION

This invention relates generally to the use of L-threonine transaldolases for producing beta-hydroxylated amino acids.

### BACKGROUND OF THE INVENTION

Aromatic non-standard amino acids (nsAAs) that contain a hydroxyl-group on the β-carbon are found naturally in many highly effective antimicrobial non-ribosomal peptides (NRPs) like vancomycin, and industrially as small molecule antibiotics and therapeutics such as amphenicols and Droxidopa. Beyond their current natural and industrial uses, some of these molecules share structural similarity with nsAAs used for genetic code expansion, a technology that has had a profound impact on chemical biology and drug development. Efficient enzymatic synthesis of stereospecific, beta-hydroxy non-standard amino acids (β-OH-nsAAs) could pave the way for inexpensive, one-pot production of chemically diverse ribosomal and non-ribosomal peptide products (FIG. 1a). Chemical diversification is valuable for drug and antibiotic development to improve cell permeability, maintain antibiotic effectiveness, and increase potency. Further, fermentative, one-pot production of β-OH-nsAAs could enable their integration into more complex products like NRPs and proteins, which are typically produced through fermentation because of their high requirements for protein synthesis and cofactor regeneration. Until recently, strategies for the biosynthesis of β-OH-nsAAs in cells were limited by restricted substrate specificity or thermodynamic favorability. Naturally, many β-OH-nsAAs are produced within NRP synthase complexes in which the active enzyme performing the beta-hydroxylation is highly specific, limiting the potential for product diversification. Alternatively, threonine aldolases (TAs) are a well-established enzyme class that exhibit substrate promiscuity and have been engineered to maintain high stereospecificity for β-OH-nsAAs production. However, TAs naturally favor the decomposition of β-OH-nsAAs and require high concentrations of glycine for efficient product formation, limiting their use in fermentation.

Fortunately, a novel enzyme class known as L-threonine transaldolases (TTAs) can perform similar chemistry with low reversibility, high stereoselectivity, and high yields. Similar to TAs, TTAs are type I pyridoxal 5′-phosphate (PLP)-dependent enzymes that catalyze the aldol condensation of L-threonine (L-Thr) with an aldehyde; however, they have higher sequence similarity to serine hydroxymethyltransferases (SHMTs) which naturally catalyze the formation of serine from glycine. Three types of TTAs have been identified: fluorothreonine transaldolases (FTases) that act on fluoroacetaldehyde; threonine:uridine 5′ aldehyde transaldolases (LipK, AmbH) that act on uridine 5′ aldehyde; and L-TTAs that act on aromatic aldehydes. In 2017, the TTA known as ObiH (or ObaG) was discovered as a part of the obafluorin biosynthesis pathway that natively catalyzed the aldol condensation of L-Thr and 4-nitrophenylacetaldehyde to produce the corresponding β-OH-nsAA (FIG. 1b). Since its discovery, ObiH (and a 99% similar variant, PsLTTA) has been characterized to have activity on over 30 aldehyde substrates as a purified enzyme and in resting cell biocatalysts, with notably little to no activity on aromatic aldehydes that contain strongly electron-donating functional groups. In these contexts, ObiH was shown to maintain low reversibility and high stereospecificity with a preference for the threo diastereomer, the isomer found in many natural products. ObiH and TTAs more broadly are a promising alternative to produce chemically diverse β-OH-nsAAs. While ObiH expresses well in heterologous hosts like Escherichia coli, it has reported limitations in substrate scope, has a low L-Thr affinity, and has not been studied in fermentative conditions. Further, the aldehyde substrates for ObiH are unstable and potentially toxic in live cell contexts.

There remains a need for identifying TTAs that are suitable for producing different beta-hydroxy non-standard amino acids (β-OH-nsAAs) than the ones that are already reported, as well as TTAs that exhibit superior catalytic properties.

### SUMMARY OF THE INVENTION

The inventors have discovered a set of hypothetical proteins or minimally characterized proteins that have limited sequence identity to known L-threonine transaldolases (TTAs) but that function as TTAs for producing a beta-hydroxy non-standard amino acid (β-OH-nsAA) in vitro or by recombinant cells (in vivo). In many respects, these new TTAs exhibit superior performance characteristics for industrial use compared to known TTAs.

A method for producing in vitro a beta-hydroxy non-standard amino acid (β-OH-nsAA) is provided. This in vitro method comprises incubating L-threonine, an aldehyde and an L-threonine transaldolase (TTA). The TTA comprises an amino acid sequence having at least 90% identity to an amino acid sequence selected from the group consisting of SEQ IDs: 1-29. As a result, a beta-hydroxy non-standard amino acid (β-OH-nsAA) is produced.

According to the in vitro method, the TTA may consist of an amino acid sequence having at least 90% identity to an amino acid sequence selected from the group consisting of SEQ ID NO: 1-29. The TTA may comprise an amino acid sequence selected from the group consisting of SEQ IDs: 1-29. The TTA may consist of an amino acid sequence selected from the group consisting of SEQ IDs: 1-29. The TTA may consist of the amino acid sequence of SEQ ID NO: 1. The TTA may consist of the amino acid sequence of SEQ ID NO: 15. The TTA may further comprise a small ubiquitin-like modifier motif (SUMO tag) (SEQ ID NO: 41).

According to the in vitro method, the aldehyde may be selected from the group consisting of aliphatic aldehydes, aromatic benzaldehydes, aromatic phenylacetaldehydes, aromatic cinnamaldehydes, and aldehydes derived from pyrimidine nucleosides. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin, protocatechualdehyde and uridine-5′-aldehyde. The aldehyde may be selected from the group consisting of 4-nitro-benzaldehyde, 2-nitro-benzaldehyde, terephthalaldehyde, phenylacetaldehyde, 4-nitro-phenylacetaldehyde and protocatechualdehyde. The aldehyde may be group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin and protocatechualdehyde.

The in vitro method may further comprise incubating a carboxylic acid and a carboxylic acid reductase (CAR) such that the aldehyde is generated from the carboxylic acid.

A method for producing a beta-hydroxy non-standard amino acid (β-OH-nsAA) by recombinant cells is also provided. This in vivo method comprises expressing a heterologous L-threonine transaldolase (TTA) by the recombinant cells. The TTA comprises an amino acid sequence having at least 90% identity to an amino acid sequence of a protein selected from the group consisting of SEQ ID NOs: 1-29. The in vivo method further comprises growing the recombinant cells in a medium. The medium comprises L-threonine and an aldehyde. As a result, a beta-hydroxy non-standard amino acid (β-OH-nsAA) is produced by the recombinant cells from the L-threonine and the aldehyde.

According to the in vivo method, the TTA may consist of an amino acid sequence having at least 90% identity to an amino acid sequence selected from the group consisting of SEQ ID NO: 1-29. The TTA may comprise an amino acid sequence selected from the group consisting of SEQ IDs: 1-29. The TTA may consist of an amino acid sequence selected from the group consisting of SEQ IDs: 1-29. The TTA may be KaTTA consisting of the amino acid sequence of SEQ ID NO: 1. The TTA may be PbTTA consisting of the amino acid sequence of SEQ ID NO: 15. The TTA may further comprise a small ubiquitin-like modifier motif (SUMO tag) (SEQ ID NO: 41).

According to the in vivo method, the aldehyde may be selected from the group consisting of aliphatic aldehydes, aromatic benzaldehydes, aromatic phenylacetaldehydes, aromatic cinnamaldehydes, and aldehydes derived from pyrimidine nucleosides. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin, protocatechualdehyde and uridine-5′-aldehyde. The aldehyde may be selected from the group consisting of 4-nitro-benzaldehyde, 2-nitro-benzaldehyde, terephthalaldehyde, phenylacetaldehyde, 4-nitro-phenylacetaldehyde and protocatechualdehyde. The aldehyde may be group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin and protocatechualdehyde.

The recombinant cells may further express a heterologous carboxylic acid reductase (CAR), the medium may further comprise a carboxylic acid, and the in vivo method further comprise generating the aldehyde by the recombinant cells from the carboxylic acid.

The recombinant cells may be of E. coli RARE strain, which is a strain of E. coli that was engineered to minimize the conversion of aromatic aldehydes to their corresponding alcohols by cellular enzymes.

### BRIEF DESCRIPTION OF THE DRAWINGS

FIGS. 1a-c illustrate threonine transaldolases as promising enzymes for biosynthesis of chemically diverse β-OH-nsAA products. (a) Cartoon depiction of potential applications for β-OH-nsAAs including diversified antibiotics, genetic code expansion, and novel non-ribosomal peptides. (b) Depiction of the natural biosynthetic gene cluster from Pseudomonas fluorescens that is responsible for the biosynthesis of the antibiotic obafluorin. One of the key enzymes in this pathway is ObiH, a threonine transaldolase (TTA). (c) Schematic of the study in Example 1: (1) ObiH activity on multiple novel candidate substrates; (2) Bioprospecting for candidate TTAs of lower protein sequence identity than previous efforts; (3) A genetic strategy to improve TTA expression; (4) The biochemical characterization of candidate TTAs in regard to substrate scope and L-Thr affinity; (5) The potential for TTA-catalyzed formation of beta hydroxylated non-standard amino acids during aerobic fermentation.

FIGS. 2a-c show use of a TTA-ADH coupled assay for screening activity of ObiH on a diverse array of aromatic aldehyde substrates. (a) Reaction schematic for coupled enzyme reaction that enables reaction monitoring at 340 nm if appropriate conditions and controls are used. Important negative controls are no addition of aldehyde (to account for the rate of threonine decomposition) and no addition of ObiH (to account for potential ADH-catalyzed reduction of the aldehyde substrate). (b) Initial rates of ObiH on aldehyde substrates relative to an L-threonine background measurement and ADH background activity on aldehydes. The horizontal line indicates the L-Thr background decomposition observed in the TTA-ADH coupled assay. Any activity greater than the dotted line and the corresponding ADH activity is considered successful activity of an ADH on that aldehyde. Experiment performed in triplicate with each replicate displayed as an individual data point and error bars represent standard deviations. (c) Chemical structures of the aldehydes investigated in Example 1. Asterisks indicate substrates never previously screened with TTAs.

FIGS. 3a-b show HPLC and LC-MS confirmation for β-OH-nsAA produced from benzaldehyde (1). (a) HPLC traces at 210 nm for the with and without TTA conditions. (b) LC-MS trace.

FIGS. 4a-b show HPLC and LC-MS confirmation for β-OH-nsAA produced from 4-nitro-benzaldehyde (2). (a) HPLC traces at 280 nm for the with and without TTA conditions. (b) LC-MS trace.

FIGS. 5a-b show HPLC and LC-MS confirmation for β-OH-nsAA produced from 2-nitro-benzaldehyde (3). (a) HPLC traces at 280 nm for the with and without TTA conditions. (b) LC-MS trace.

FIGS. 6a-b show HPLC and LC-MS confirmation for β-OH-nsAA produced from 4-amino-methyl-benzaldehyde (4). (a) HPLC traces at 280 nm for the with and without TTA conditions. (b) LC-MS trace.

FIG. 7a shows LC-MS confirmation for β-OH-nsAA produced from 2-amino-benzaldehyde (6).

FIGS. 8a-b show HPLC and LC-MS confirmation for β-OH-nsAA produced from terephthalaldehyde (7). (a) HPLC traces at 250 nm for the with and without TTA conditions. (b) LC-MS trace.

FIG. 9a shows HPLC confirmation for β-OH-nsAA produced from 4-methoxybenzaldehyde (9) at 210 nm via HPLC traces at 210 nm for with and without TTA conditions.

FIGS. 10a-b show HPLC and LC-MS β-OH-nsAA produced from confirmation for 4-biphenylcarboxaldehyde (10). (a) HPLC traces at 280 nm for the with and without TTA conditions. (b) LC-MS trace.

FIGS. 11a-b show HPLC and LC-MS confirmation for β-OH-nsAA produced from 2-napthaldehyde (11). (a) HPLC traces at 280 nm for the with and without TTA conditions. (b) LC-MS trace.

FIG. 12a shows LC-MS confirmation for β-OH-nsAA produced from phenylacetaldehyde (14).

FIG. 13a shows LC-MS confirmation for β-OH-nsAA produced from 4-nitro-phenylacetaldehyde (15).

FIG. 14a-b shows HPLC and LC-MS confirmation for β-OH-nsAA produced from 2-nitrophenylacetaldehyde (16). (a) HPLC traces at 280 nm for the with and without TTA conditions. (b) LC-MS trace.

FIGS. 15a-c show bioprospecting and expression of putative threonine transaldolases. (a) A Protein Sequence Similarity Network (SSN) containing 859 sequences related to ObiH, LipK, and FTase with selected putative TTAs highlighted in yellow. Existing enzymes characterized in the literature are highlighted in teal except those found in the largest cluster which contains many SHMTs. (b) Sequence identity matrix for all selected TTAs in this study. (c) Western blot of all TTAs with the tagged and untagged TTA constructs demonstrating improved expression of TTAs with a SUMO solubility tag. Proteins that contain an N-terminal SUMO tag followed by a TEV protease cleavage site, and no other changes, are shown in lanes indicated by the ‘s’.

FIGS. 16a-d show characterization of putative threonine transaldolases. (a) Screen of all purified TTAs using TTA-ADH assay on 2-nitro-benzaldehyde. Experiment performed in triplicate with each replicate as an individual point. Error bars represent standard deviations. (b) Apparent L-Thr K_{M} and k_{cat} measurements for TTAs that exhibited activity greater than or equal to ObiH calculated using non-linear regression. Parenthetical values represent the 95% confidence interval. (c) Heatmap showing initial rates for six active TTAs against multiple aromatic aldehyde substrates. (d) Multi-sequence alignment of the predicted conserved catalytic residues for the six active TTAs. (e) Superimposed structure and predicted structure illustrating the Tyr55-Pro71 loop region of ObiH compared to the predicted equivalent region for PbTTA. The ObiH loop region is in a light gray with the PLP highlighted in black indicating the region of the active site. The PbTTA loop region is indicated with a dark gray.

FIG. 17 shows the diastereomeric excess for the β-OH-nsAA produced from 2-nitro-benzaldehyde for all active enzymes. (a) The de % for the threo isomer for each of the active enzymes with reaction conditions as specified in the main text and quenched after 20 h. de % was calculated as follows (threo−erythro)/(threo+erythro). (b) HPLC traces for ObiH and PbTTA as well as the chemically synthesized standard to demonstrate how we identified the diastereomers.

FIG. 18 shows novel activity of PbTTA and KaTTA on vanillin and protocatechualdehyde. (a) Heatmap for a collection of vanillin and protocatechualdehyde across all active TTAs demonstrating the activity of PbTTA and s-KaTTA on novel substrates vanillin and protocatechualdehyde.

FIGS. 19a-f show biosynthesis of β-OH-nsAAs in metabolically active cells during aerobic fermentation. (a) Schematic of β-OH-nsAA biosynthesis with supplemented aldehyde in a wild-type E. coli strain. (b) β-OH-nsAA titer measured after 20 h for s-ObiH, s-BuTTA, and s-PbTTA with 0, 10, and 100 mM of L-Thr supplemented. (c) Schematic of β-OH-nsAA biosynthesis with genomic modifications to improve aldehyde stabilization. (d) β-OH-nsAA titer measured after 20 h for s-ObiH, s-BuTTA, and s-PbTTA with 0, 10, and 100 mM of L-Thr supplemented. (e) Schematic of biosynthesis of β-OH-nsAA from an acid precursor when the TTA is coupled with a CAR in the RARE strain. (f) β-OH-nsAA peak area for 4-formyl-β-OH-phenylalanine from 4-formyl benzoic acid and terephthalaldehyde within the RARE strain with pACYC-NiCAR and pZE-s-PbTTA for the coupled production and RARE with pACYC-s-PbTTA, otherwise. All experiments performed with technical triplicates. Each replicate is represented as its own data point with error bars representing standard deviations.

FIGS. 20a-d show novel activity of CARs and PbTTA to produce 4-azido-β-OH-phenylalanine. (a) Reaction scheme for the conversion of 4-azido-benzoic acid to 4-azido-β-OH-phenylalanine. (b) Initial rate of NADPH depletion measured for three purified CARs when provided the previously unreported candidate substrate of 4-azido benzoic acid. (c) β-OH-nsAA production measured by peak area for an in vitro coupled assay with the specified CAR and PbTTA. (d) β-OH-nsAA production measured by peak area in aerobically cultivated cells of the E. coli RARE strain transformed to express each CAR on a pZE vector and pACYC-s-PbTTA. Cultures were supplemented with 4-azido-benzoic acid during mid-exponential phase and sampled after 20 h of growth. Experiments performed in technical triplicate with each replicate represented. Error bars are standard deviations.

FIG. 21 shows HPLC confirmation for β-OH-nsAA produced from 4-azido-carboxylic acid at 280 and 250 nm via HPLC traces for with and without CAR and TTA conditions.

### DETAILED DESCRIPTION OF THE INVENTION

The present invention provides a method for producing beta-hydroxy non-standard amino acids (β-OH-nsAAs) from L-threonine and an aldehyde in the presence of an L-threonine transaldolase (TTA). The invention is based on the inventors' surprising discovery of the specificity of the TTA enzyme class by characterizing 12 candidate TTA gene products across a wide range (20-80%) of sequence identities. The inventor has improved the accuracy of a high throughput coupled enzyme activity for TTA activity. The inventors have also found that the addition of a solubility tag substantially enhanced the soluble protein expression level within this difficult to express enzyme family, with improvements observed for nine putative TTAs. Using the coupled enzyme assay, the inventors have identified six TTAs including one that exhibits broader substrate scope, two-fold higher L-Threonine (L-Thr) affinity, and five-fold faster initial reaction rates. Remarkably, these superior TTAs included sequences that contained less than 30% identity to ObiH. The inventors have harnessed these TTAs for first-time bioproduction of β-OH-nsAAs that contain handles for bio-orthogonal conjugation from supplemented precursors during aerobic fermentation of engineered Escherichia coli cells, where higher affinity of the TTA for L-Thr increased titer was observed. Overall, the inventors have revealed an unexpectedly high level of sequence diversity and broad substrate specificity in an enzyme family whose members play key roles in the biosynthesis of therapeutic natural products that could benefit from chemical diversification.

The term “L-threonine transaldolase (TTA)” as used herein refers to an enzyme that performs the aldol condensation of L-threonine and aldehyde to produce beta-hydroxy non-standard amino acid (β-OH-nsAA) and acetaldehyde as a co-product of the reaction, which makes the aldol condensation reaction more favorable than for the related class of enzymes known as threonine aldolases.

The term “beta-hydroxy non-standard amino acid (β-OH-nsAA)” as used herein refers to an amino acid that contains a hydroxy group (OH) covalently bound to the beta-carbon.

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28), EbTTA (SEQ ID NO: 29), ObiH (SEQ ID NO: 30), PiTTA (SEQ ID NO: 31), BsTTA (SEQ ID NO: 32), CsTTA (SEQ ID NO: 33), BuTTA (SEQ ID NO: 34), StTTA (SEQ ID NO: 35), TmTTA (SEQ ID NO: 36), RaTTA (SEQ ID NO: 37), SnTTA (SEQ ID NO: 38), NoTTA (SEQ ID NO: 39) and DbTTA (SEQ ID NO: 40). The TTA may further comprise a small ubiquitin-like modifier motif (SUMO tag) (SEQ ID NO: 41) (Tables 6-8).

The TTA may comprise the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28), EbTTA (SEQ ID NO: 29), ObiH (SEQ ID NO: 30), PiTTA (SEQ ID NO: 31), BsTTA (SEQ ID NO: 32), CsTTA (SEQ ID NO: 33), BuTTA (SEQ ID NO: 34), StTTA (SEQ ID NO: 35), TmTTA (SEQ ID NO: 36), RaTTA (SEQ ID NO: 37), SnTTA (SEQ ID NO: 38), NoTTA (SEQ ID NO: 39) and DbTTA (SEQ ID NO: 40). The TTA may further comprise a small ubiquitin-like modifier motif (SUMO tag) (SEQ ID NO: 41).

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29). The TTA may further comprise a small ubiquitin-like modifier motif (SUMO tag) (SEQ ID NO: 41).

The TTA may comprise the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29). The TTA may further comprise a small ubiquitin-like modifier motif (SUMO tag) (SEQ ID NO: 41).

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14) and StdTTA2 (SEQ ID NO: 15). The TTA may further comprise a small ubiquitin-like modifier motif (SUMO tag) (SEQ ID NO: 41).

The TTA may comprise the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14) and StdTTA2 (SEQ ID NO: 15). The TTA may further comprise a small ubiquitin-like modifier motif (SUMO tag) (SEQ ID NO: 41).

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29). The TTA may further comprise a small ubiquitin-like modifier motif (SUMO tag) (SEQ ID NO: 41).

The TTA may comprise the amino acid sequence of a protein selected from the group consisting of PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29). The TTA may further comprise a small ubiquitin-like modifier motif (SUMO tag) (SEQ ID NO: 41).

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of KaTTA (SEQ ID NO: 1). The TTA may further comprise a small ubiquitin-like modifier motif (SUMO tag) (SEQ ID NO: 41).

The TTA may comprise the amino acid sequence of KaTTA (SEQ ID NO: 1). The TTA may further comprise a small ubiquitin-like modifier motif (SUMO tag) (SEQ ID NO: 41).

The TTA may comprise an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of PbTTA (SEQ ID NO: 16). The TTA may further comprise a small ubiquitin-like modifier motif (SUMO tag) (SEQ ID NO: 41).

The TTA may comprise the amino acid sequence of PbTTA (SEQ ID NO: 16). The TTA may further comprise a small ubiquitin-like modifier motif (SUMO tag) (SEQ ID NO: 41).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28), EbTTA (SEQ ID NO: 29), ObiH (SEQ ID NO: 30), PiTTA (SEQ ID NO: 31), BsTTA (SEQ ID NO: 32), CsTTA (SEQ ID NO: 33), BuTTA (SEQ ID NO: 34), StTTA (SEQ ID NO: 35), TmTTA (SEQ ID NO: 36), RaTTA (SEQ ID NO: 37), SnTTA (SEQ ID NO: 38), NoTTA (SEQ ID NO: 39) and DbTTA (SEQ ID NO: 40).

The TTA may consist of the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28), EbTTA (SEQ ID NO: 29), ObiH (SEQ ID NO: 30), PiTTA (SEQ ID NO: 31), BsTTA (SEQ ID NO: 32), CsTTA (SEQ ID NO: 33), BuTTA (SEQ ID NO: 34), StTTA (SEQ ID NO: 35), TmTTA (SEQ ID NO: 36), RaTTA (SEQ ID NO: 37), SnTTA (SEQ ID NO: 38), NoTTA (SEQ ID NO: 39) and DbTTA (SEQ ID NO: 40).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29).

The TTA may consist of the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14), StdTTA2 (SEQ ID NO: 15), PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14) and StdTTA2 (SEQ ID NO: 15).

The TTA may consist of the amino acid sequence of a protein selected from the group consisting of KaTTA (SEQ ID NO: 1), ScTTA1 (SEQ ID NO: 2), SanTTA (SEQ ID NO: 3), ScTTA2 (SEQ ID NO: 4), KmTTA (SEQ ID NO: 5), SauTTA (SEQ ID NO: 6), StTTA2 (SEQ ID NO: 7), SpTTA (SEQ ID NO: 8), StTTA3 (SEQ ID NO: 9), StTTA4 (SEQ ID NO: 10), SRTTA (SEQ ID NO: 11), SuTTA (SEQ ID NO: 12), SSTTA (SEQ ID NO: 13), StdTTA1 (SEQ ID NO: 14) and StdTTA2 (SEQ ID NO: 15).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of a protein selected from the group consisting of PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29).

The TTA may consist of the amino acid sequence of a protein selected from the group consisting of PbTTA (SEQ ID NO: 16), StnTTA (SEQ ID NO: 17), PaTTA (SEQ ID NO: 18), GabTTA (SEQ ID NO: 19), FeTTA (SEQ ID NO: 20), FITTA (SEQ ID NO: 21), FpTTA (SEQ ID NO: 22), ScTTA (SEQ ID NO: 23), StTTA5 (SEQ ID NO: 24), LSTTA (SEQ ID NO: 25), SaTTA (SEQ ID NO: 26), DbTTA2 (SEQ ID NO: 27), RbTTA (SEQ ID NO: 28) and EbTTA (SEQ ID NO: 29).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of KaTTA (SEQ ID NO: 1).

The TTA may consist of the amino acid sequence of KaTTA (SEQ ID NO: 1).

The TTA may consist of an amino acid sequence having at least about 20%, 30%, 40%, 50%, 60%, 70%, 80%, 90%, 95% or 99%, or about 20-80%, 20-90%, 20-95%, 20-99%, 30-80%, 30-90%, 30-95%, 30-99%, 50-80%, 50-90%, 50-95%, 30-99%, 80-90%, 80-95%, 90-99%, 90-95% or 90-99% identity to the amino acid sequence of PbTTA (SEQ ID NO: 16).

The TTA may consist of the amino acid sequence of PbTTA (SEQ ID NO: 16).

The present invention provides a method for producing in vitro a beta-hydroxy non-standard amino acid (β-OH-nsAA). This in vitro method comprises incubating L-threonine, an aldehyde, and an L-threonine transaldolase (TTA) such that a beta-hydroxy non-standard amino acid (β-OH-nsAA) is produced.

According to the in vitro method, the aldehyde may be selected from the group consisting of aliphatic aldehydes, aromatic benzaldehydes, aromatic phenylacetaldehydes, aromatic cinnamaldehydes, and aldehydes derived from pyrimidine nucleosides. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin, protocatechualdehyde and uridine-5′-aldehyde. The aldehyde may be selected from the group consisting of 4-nitro-benzaldehyde, 2-nitro-benzaldehyde, terephthalaldehyde, phenylacetaldehyde, 4-nitro-phenylacetaldehyde and protocatechualdehyde. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin and protocatechualdehyde.

The in vitro method may further comprise incubating a carboxylic acid and a carboxylic acid reductase (CAR) such that the aldehyde is generated from the carboxylic acid.

A method for producing a beta-hydroxy non-standard amino acid (β-OH-nsAA) by recombinant cells is also provided. This in vivo method comprises expressing a heterologous L-threonine transaldolase (TTA) by the recombinant cells; and growing the recombinant cells in a medium. The medium may comprise L-threonine and an aldehyde. As a result, a beta-hydroxy non-standard amino acid (β-OH-nsAA) is produced by the recombinant cells from the L-threonine and the aldehyde.

According to the in vivo method, the aldehyde may be selected from the group consisting of aliphatic aldehydes, aromatic benzaldehydes, aromatic phenylacetaldehydes, aromatic cinnamaldehydes, and aldehydes derived from pyrimidine nucleosides. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin, protocatechualdehyde and uridine-5′-aldehyde. The aldehyde may be selected from the group consisting of 4-nitro-benzaldehyde, 2-nitro-benzaldehyde, terephthalaldehyde, phenylacetaldehyde, 4-nitro-phenylacetaldehyde and protocatechualdehyde. The aldehyde may be selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin and protocatechualdehyde.

Where the recombinant cells further express a heterologous carboxylic acid reductase (CAR) and the medium further comprises a carboxylic acid, the in vivo method may further comprise generating the aldehyde by the recombinant cells from the carboxylic acid.

According to the in vivo method, the recombinant cells are of E. coli RARE strain.

### Example 1. L-Threonine Transaldolases for Enhanced Biosynthesis of Beta-Hydroxylated Amino Acids

To address the limitations associated with ObiH, the inventors sought to further characterize ObiH, the natural space of sequences that resemble TTAs, and the activity of members of this enzyme family when expressed within cells grown under aerobic culturing conditions. At the outset of our study, ObiH, PsLTTA (a 99% similar homolog) and a promiscuous FTase (FTaseMA), were the only TTAs characterized to act on aromatic aldehydes. Furthermore, early studies did not report testing of some valuable aldehydes such as those that contain large hydrophobic moieties for cell penetration(Kalafatovic & Giralt, 2017) or handles for bio-orthogonal click chemistry. Additionally, the reported L-Thr K_{M} for ObiH (40.2±3.8 mM) is incompatible with natural E. coli L-Thr concentrations (normally <200 μM). Interestingly, LipK and FTaseMA were reported to have lower L-Thr K_{M} (29.5 mM and 1.18 mM, respectively), but both are reported to have poor soluble expression in E. coli. Together, these observations offer promise for identifying a natural TTA that accepts a broad aldehyde substrate scope, has a high L-Thr affinity, and is active in heterologous host E. coli. Very few TTAs have been identified in nature, and many are likely annotated as hypothetical proteins or SHMTs based on their primary amino acid sequence.

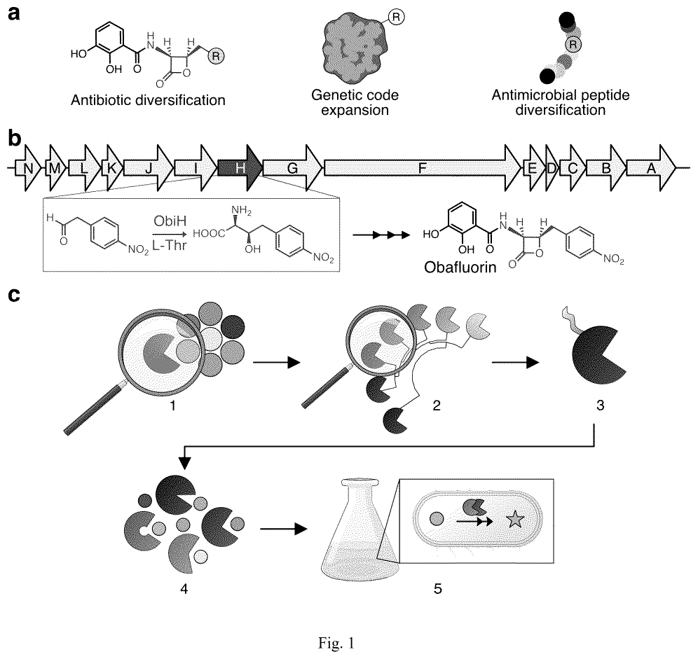

In this study, the inventors tackled each of the challenges associated with engineering in vivo biosynthesis of β-OH-nsAAs in a model heterologous host: low L-Thr affinity, protein solubility in E. coli, and aldehyde substrate stability (FIG. 1c). To enable rapid screening of many aldehydes and enzymes, the inventors first optimized a high throughput in vitro assay for characterization of TTAs on diverse aldehydes and demonstrated activity of ObiH on aldehydes with bioconjugatable handles. Then to explore the natural TTA sequence space, the inventors generated a sequence similarity network (SSN) of enzymes with high similarity to ObiH, FTase, and LipK. After appending a solubility tag to many distantly related TTAs, the inventors observed dramatically improved enzyme expression and then identified previously unreported TTAs that exhibit higher L-Thr affinity, faster reaction kinetics, and broad substrate scope. Remarkably, one of the best TTAs, which is annotated as a hypothetical protein, shares only 27.2% sequence identity with ObiH. Next, the inventors biosynthesized β-OH-nsAAs with the novel TTAs in an engineered chassis for aldehyde stabilization and coupled the TTAs to a carboxylic acid reductase (CAR) to limit toxic aldehyde accumulation. Finally, the inventors demonstrated novel activity of several CARs and a TTA in vitro and in growing cells to produce 4-azido-β-OH-phenylalanine (4-azido-β-OH-Phe), an nsAA with a well-established handle for bio-orthogonal conjugation. The work presented here brings the field closer to achieving one-pot synthesis of chemically diverse peptides and proteins through biosynthesis of diverse β-OH-nsAAs in cells growing in aerobic conditions after supplementation with aldehyde or acid precursors.

### 1. Materials and Methods

### 1.1 Strains and Plasmids

Escherichia coli strains and plasmids used are listed in Table 1. Molecular cloning and vector propagation were performed in DH5α. Polymerase chain reaction (PCR) based DNA replication was performed using KOD XTREME™ Hot Start Polymerase for plasmid backbones or using KOD Hot Start Polymerase otherwise. Cloning was performed using Gibson Assembly with constructs and oligos for PCR amplification shown in Table 2. Genes were purchased as G-Blocks or gene fragments from Integrated DNA Technologies (IDT) or Twist Bioscience and were optimized for E. coli K12 using the IDT Codon Optimization Tool with sequences shown in Table 3.

### 1.2 Chemicals

The following compounds were purchased from MilliporeSigma: kanamycin sulfate, dimethyl sulfoxide (DMSO), potassium phosphate dibasic, potassium phosphate monobasic, magnesium chloride, calcium chloride dihydrate, imidazole, glycerol, beta-mercaptoethanol, sodium dodecyl sulfate, lithium hydroxide, boric acid, Tris base, glycine, HEPES, L-threonine, L-serine, adenosine 5′-triphosphate disodium salt hydrate, pyridoxal 5′-phosphate hydrate, benzaldehyde, 4-nitro-benzaldehyde, 4-amine-methyl-benzaldehyde, 4-formyl benzoic acid, 4-methoxybenzaldehyde, 2-naphthaldehyde, 4-formyl boronic acid, NADH, phosphite, Boc-glycine-OH, trimethylacetyl chloride, (1R,2R)-2-(Methylamino)-1,2-diphenylethanol, trifluoroacetic acid, alcohol dehydrogenase from S. cerevisiae, and KOD XTREME™ Hot Start and KOD Hot Start polymerases. Lithium bis(trimethylsilyl)amide, 4-dimethyl-amino-benzaldehyde, and 2-amino-benzaldehyde were purchased from Acros. D-glucose, 2-nitro-benzaldehyde, 4-biphenyl-carboxaldehyde, terephthalaldehyde, and 4-azido-benzoic acid were purchased from TCI America. Agarose, Laemmli SDS sample reducing buffer, 4-tert-butyl-benzaldehyde, phenylacetaldehyde, and ethanol were purchased from Alfa Aesar. 2-nitro-phenylacetaldehyde and 4-nitro-phenylacetaldehyde were purchased from Advanced Chem Block. Anhydrotetracycline (aTc) was purchased from Cayman Chemical. Hydrochloric acid was purchased from RICCA. Acetonitrile, methanol, sodium chloride, LB Broth powder (Lennox), LB Agar powder (Lennox), AMERSHAM™ ECL Prime chemiluminescent detection reagent, bromophenol blue, and THERMO SCIENTIFIC™ SPECTRA™ Multicolor Broad Range Protein Ladder were purchased from Fisher Chemical. NADPH was purchased through ChemCruz. A MOPS EZ rich defined medium kit and components for was purchased from Teknova. Trace Elements A was purchased from Corning. Taq DNA ligase was purchased from GoldBio. PHUSION™ DNA polymerase and T5 exonuclease were purchased from New England BioLabs (NEB). SYBR™ Safe DNA gel stain was purchased from Invitrogen. HRP-conjugated 6*His His-Tag Mouse McAB was obtained from Proteintech.

### 1.3 Overexpression and Purification of Threonine Transaldolases

A strain of E. coli BL21 transformed with a pZE plasmid encoding expression of a TTA with a hexahistidine tag or a hexahistidine-SUMO tag at the N-terminus (P1-P26) was inoculated from frozen stocks and grown to confluence overnight in 5 mL LBL containing kanamycin (50 μg/mL). Confluent cultures were used to inoculate 250-400 mL of experimental culture of LBL supplemented with kanamycin (50 μg/mL). The culture was incubated at 37° C. until an OD₆₀₀ of 0.5-0.8 was reached while in a shaking incubator at 250 RPM. TTA expression was induced by addition of anhydrotetracycline (0.2 nM) and cultures were incubated shaking at 250 RPM at either 18° C. for 24 h, 30° C. for 5 h then 18° C. for 20 h or 30° C. for 24 h. Cells were centrifuged using an Avanti J-15R refrigerated Beckman Coulter centrifuge at 4° C. at 4,000 g for 15 min. Supernatant was then aspirated and pellets were resuspended in 8 mL of lysis buffer (25 mM HEPES, 10 mM imidazole, 300 mM NaCl, 400 μM PLP, 10% glycerol, pH 7.4) and disrupted via sonication using a QSonica Q125 sonicator with cycles of 5 s at 75% amplitude and 10 s off for 5 min. The lysate was distributed into microcentrifuge tubes and centrifuged for 1 h at 18,213×g at 4° C. The protein-containing supernatant was then removed and loaded into a HisTrap Ni-NTA column using an ÄKTA™ Pure GE FPLC system. Protein was washed with 3 column volumes (CV) at 60 mM imidazole and 4 CV at 90 mM imidazole. TTA was eluted in 250 mM imidazole in 1.5 mL fractions over 6 CV. Samples from selected fractions were denatured in Lamelli SDS reducing sample buffer (62.5 mM Tris-HCl, 1.5% SDS, 8.3% glycerol, 1.5% beta-mercaptoethanol, 0.005% bromophenol blue) for 10 min at 95° C. and subsequently run on an SDS-PAGE gel with a THERMO SCIENTIFIC™ PAGERULER™ Prestained Plus ladder to identify protein containing fractions and confirm their size. The TTA containing fractions were combined applied to an AMICON™ column (10 kDa MWCO) and the buffer was diluted 1,000× into a 25 mM HEPES, 400 μM PLP, 10% glycerol buffer. This same method was used for purification of the CAR enzymes, E. coli pyrophosphatase, E. coli ADHs, and the phosphite dehydrogenase.

### 1.4 Threonine Transaldolase Expression Testing

To test expression of the threonine transaldolase library, 5 mL cultures of MAJ14-26 and MAJ53-65 were inoculated in 5 mL cultures of LBL containing 50 μg/mL kanamycin and then grown shaking at 250 RPM at 37° C. until mid-exponential phase (OD=0.5-0.8). At this time, cultures were induced via addition of 0.2 nM aTc and then grown shaking at 250 RPM at 30° C. for 24 h. After this time, 1 mL of cells was mixed with 0.05 mL of glass beads and then vortexed using a VORTEX-GENIE® 2 for 15 min. After this time, the lysate was centrifuged at 18,213 g at 4° C. for 30 min. Lysate was denatured as described for the overexpression and then subsequently run on an SDS-PAGE gel with THERMO SCIENTIFIC™ SPECTRA™ Multicolor Broad Range Protein Ladder and then analyzed via western blot with an HRP-conjugated 6*His His-Tag Mouse McAB primary antibody. The blot was visualized using an AMERSHAM™ ECL Prime chemiluminescent detection reagent.

### 1.5. In Vitro Enzyme Activity Assay

### 1.5.1 TTA-ADH

High-throughput screening of purified TTAs was performed with a TTA-ADH coupled assay using purified TTA and commercially available alcohol dehydrogenase from S. cerevisiae purchased from MilliporeSigma. Aldehyde stocks were prepared in 50-100 mM solutions in DMSO or acetonitrile. Reaction mixtures were prepared in a 96-well plate with 100 μL of 100 mM phosphate buffer pH 7.5, 0.5 mM NADH, 0.4 mM PLP, 15 mM MgCl₂, and 100 mM L-Thr with the addition of 0.25 mM to 1 mM aldehyde depending on the background absorbance at 340 nm (Table 4), 10 U ScADH, and 0.25 μM purified TTA unless otherwise specified. Reactions were initiated with the addition of enzyme. Reaction kinetics were observed for 20-60 min in a SPECTRAMAX® i3× microplate reader at 30° C. with 5 sec of shaking between reads with the high orbital shake setting. The following controls were included for every assay: reaction mixture without aldehyde, without TTA, and without enzyme (TTA or ADH). Rates were calculated by identifying the linear region at the beginning of the kinetic run and converting the depletion in absorbance to the depletion of mM NADH using an NADH standard curve.

### 1.5.2 CAR-TTA

In vitro CAR activity assays were performed as previously reported (Gopal et al. biorxiv, 2022) using 2 mM NADPH and 2 mM ATP, 20 mM MgCl₂, and 0.75 μM CAR and E. coli pyrophosphatase. For in vitro coupling with the CAR and TTA, the same in vitro CAR assay was performed with the addition of 2 μM TTA, 0.4 mM PLP, and 100 mM L-Thr; however, rather than monitoring the reaction with the plate reader, the plate was left shaking at 1000 RPM with an orbital radius of 1.25 mm at 30° C. overnight. The reaction was then quenched after 20 h with 100 μL of 3:1 methanol:2 M HCl. The supernatant was then separated from the protein precipitate using centrifugation and analyzed via HPLC.

### 1.6 HPLC Analysis

Metabolites of interest were quantified via high-performance liquid chromatography (HPLC) using an Agilent 1260 Infinity model equipped with a Zorbax Eclipse Plus-C18 column. To quantify aldehyde and β-OH-nsAAs, an initial mobile phase of solvent A/B=95/5 was used (solvent A, water+0.1% TFA; solvent B, acetonitrile+0.1% TFA) and maintained for 5 min. A gradient elution was performed (A/B) as follows: gradient from 95/5 to 50/50 for 5-12 min, gradient from 50/50 to 0/100 for 12-13 min, and gradient from 0/100 to 95/5 for 13-14 min. A flow rate of 1 mL min-1 was maintained, and absorption was monitored at 210, 250 and 280 nm.

### 1.7 Culture Conditions

For screening TTA activity in aerobically growing cells, we inoculated strains transformed with plasmids expressing TTAs into 300 μL volumes of MOPS EZ Rich media in a 96-deep-well plate with appropriate antibiotic added to maintain plasmids (50 μg/mL kanamycin (Kan)). Cultures were incubated at 37° C. with shaking at 1000 RPM and an orbital radius of 1.25 mm until an OD₆₀₀ of 0.5-0.8 was reached. OD₆₀₀ was measured using a SPECTRAMAX® i3× plate reader. At this point, the TTAs were induced with addition of 0.2 nM aTc for TTA expression. Then, 2 h following induction of the TTAs, 1 mM aldehyde was added to the culture. Cultures were then incubated over 20 h at 30° C. with metabolite concentration measured via supernatant sampling and submission to HPLC.

For the CAR-TTA coupled assay, the strains transformed with a plasmid expressing a TTA and a second plasmid expressing a CAR were grown under identical conditions with the addition of 34 μg/mL chloramphenicol (Cm) to maintain the additional plasmid. Further, 0.2 nM aTc and 1 mM IPTG were added to induce protein expression and 2 mM aldehyde, or acid was added at the time of induction. Following induction, the cultures were grown for 20 h at 30° C. while shaking at 1000 RPM with product concentrations measured via supernatant sampling and submission to HPLC.

### 1.8 Computational Methods

### 1.8.1 Creation of Protein Sequence Similarity Network (SSN)

Using NCBI BLAST, the 500 most closely related sequences as measured by BLASTP alignment score were obtained from three characterized threonine transaldolases, FTase, LipK, and ObiH. After deleting duplicate sequences, 1195 unique sequences were obtained, which were then submitted to the Enzyme Function Initiative-Enzyme Similarity Tool (EFI-EST) to generate a sequence similarity network (SSN). Sequences exhibiting greater than 95% similarity were grouped into single nodes, resulting in 859 unique nodes and a minimum alignment score of 85 was selected for node edges. The SSN was visualized and labeled in Cytoscape using the yFiles Organic Layout.

### 1.8.2 Sequence Alignment

Multiple sequence alignments were performed using ClustalOmega alignment within JalView using the “dealign” setting and otherwise default settings of one for max guide tree iterations, and one for number of iterations (combined). The sequence identity matrix was generated using the online interface for the Multiple Sequence Alignment tool from ClustalOmega.

### 1.8.3 Structure Prediction

Structures of the putative TTAs were produced using AlphaFold2 CoLab notebook (Mirdita et al. Nat Methods, 2022) using the provided default settings with no template, the MMseqs2 (UniRef+Environmental) for multi-sequence alignment, unpaired+paired mode, auto for model_type and 3 for num_recycles. We then moved forward with the model ranked the highest. We performed the alignment of chains A and B from the crystal structure of ObiH (PDB ID: 7K34) and the AlphaFold model for PbTTA using the align command in PyMOL with all default settings. The same alignment protocol was implemented for aligning the AlphaFold2 models of putative TTAs with and without the SUMO tag.

1.9 Mass Spectrometry Confirmation of β-OH nsAAs Using In Vitro TTA-ADH Coupled Assay

Mass spectrometry (MS) measurements for small molecule metabolites were submitted to a Waters AQUITY Arc UPLC H-Class with a diode array coupled to a Waters AQUITY QDa Mass Detector. Metabolite compounds were analyzed using a Waters Cortecs UPLC C18 column with an initial mobile phase of solvent A/B=95/5 (solvent A, water, 0.1% formic acid; solvent B, acetonitrile, 0.1% formic acid) for 5 min with a gradient elution from (A/B) 95/5 to 10/90 for 5-7 min, an isocratic flow at 10/90 for 7-10 min, then gradient from 10/90 to 95/5 for 10-10.5 min and a final isocratic step for 10-12 min. Flow rate was maintained at 1 mL min-1.

### 2. Results

### 2.1 Optimizing a High-Throughput Assay for Screening TTA Activity on Diverse Aldehydes

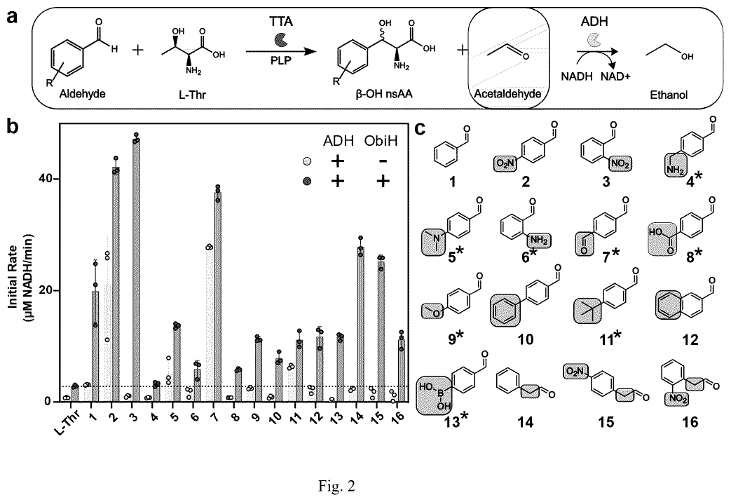

To expand our understanding of the TTA enzyme class, we wanted a high-throughput method for rapid screening of multiple enzymes and candidate aldehyde substrates. We began by analyzing a previously reported coupled enzyme assay (FIG. 2a) based on the addition of alcohol dehydrogenase (ADH), which consumes NADH to reduce the co-product acetaldehyde in a manner that can be monitored at 340 nm. Unfortunately, this coupled assay for TTA activity suffers from false positives and confounding variables which we sought to address. First, the commercially available ADH from Saccharomyces cerevisiae exhibits activity on many aromatic aldehydes which were candidate substrates for ObiH. We briefly investigated other alcohol dehydrogenases from E. coli to limit this undesired activity and remain active on the desired acetaldehyde co-product, but we did not identify a better alternative. Second, the characterized TTAs are known to catalyze the decomposition of L-Thr in the absence of an aldehyde substrate, which is an undesired reaction that also generates an acetaldehyde co-product. Another limitation of the TTA-ADH coupled assay is that many of the aromatic aldehyde candidate substrates absorb at the same measurement wavelength (Table 4). Thus, we minimized the impact of the false positives, spectral overlap, and other confounding variables by tuning enzyme and aldehyde concentrations and monitoring the undesired reactions with two controls: (1) lacking aldehyde substrate (“L-Thr”) and (2) lacking TTA (“no TTA”) where only the ADH and substrate are present. Then, we validated the TTA-ADH coupled assay by performing HPLC analysis, using the chemically synthesized β-OH-nsAA standard for the assumed product from 3, over a time course where we observed that the addition of the ScADH improves reaction rates three-fold. As previously reported by others, we were also able to improve β-OH-nsAAs yields when using the ScADH coupled to a co-factor regeneration system. As the last step of verification, we screened the TTA-ADH coupled assay with ObiH before and after photo-treatment, we observed no differences in reaction rate and continued to assay the TTAs without photo-treatment.

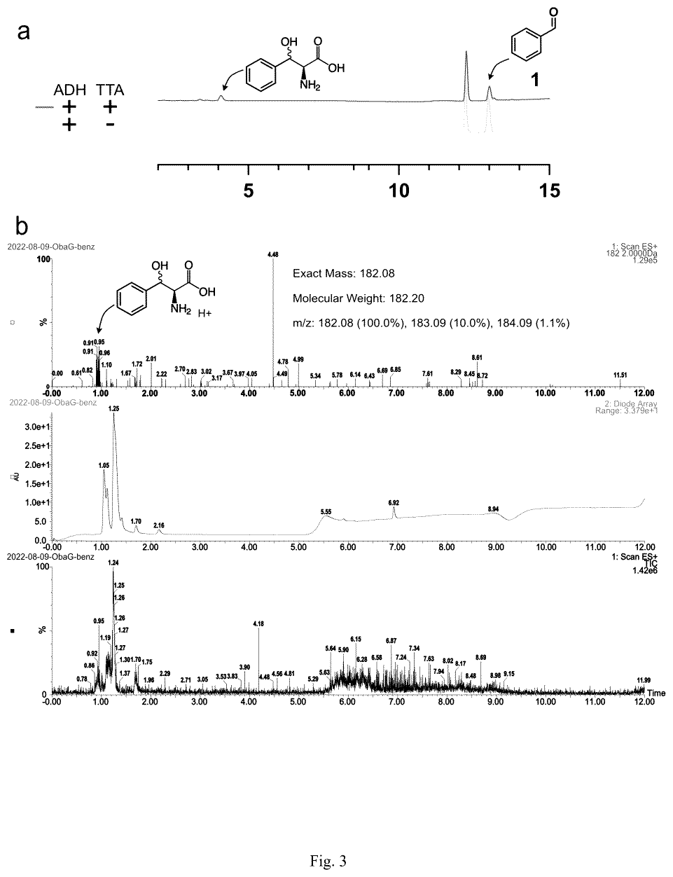

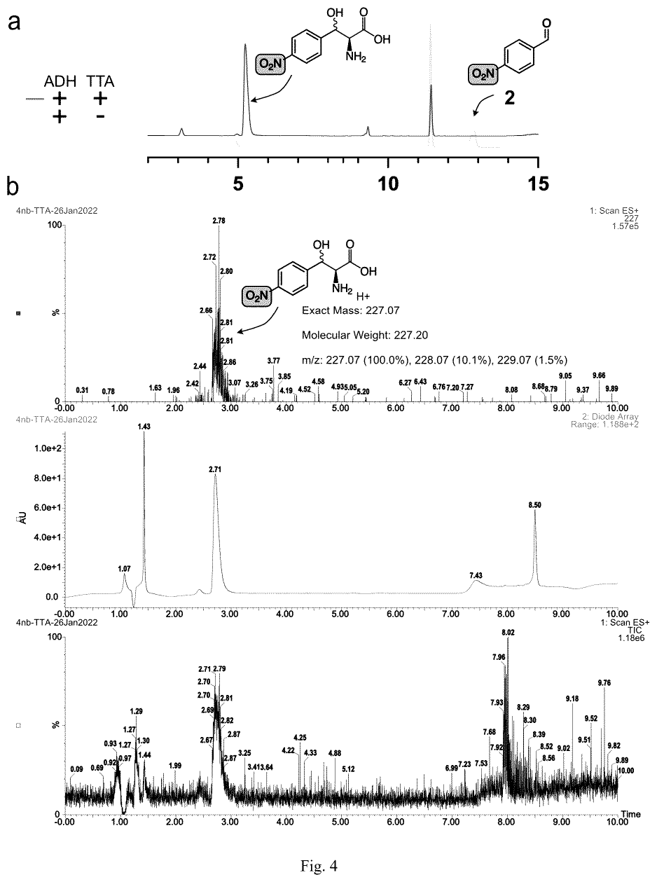

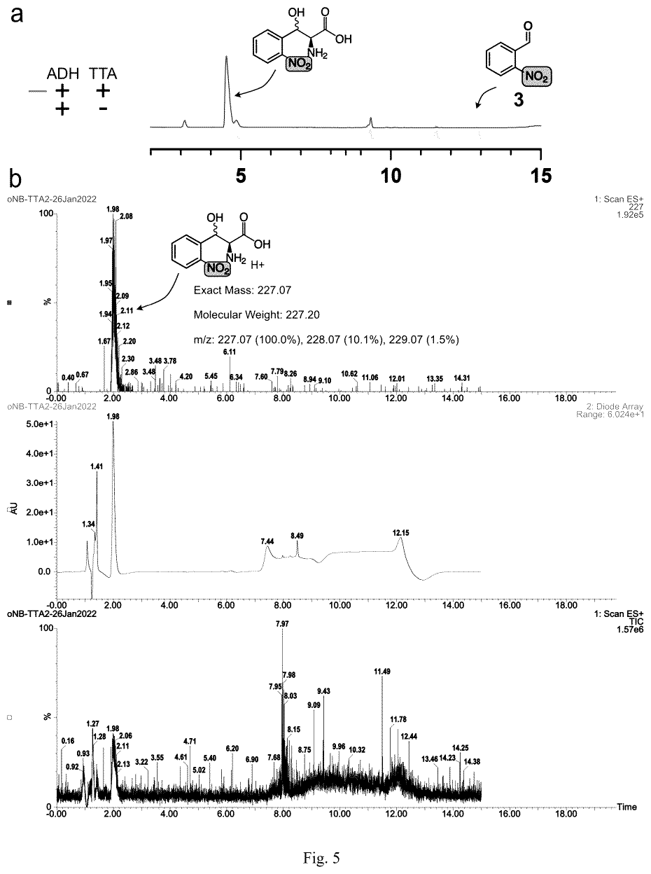

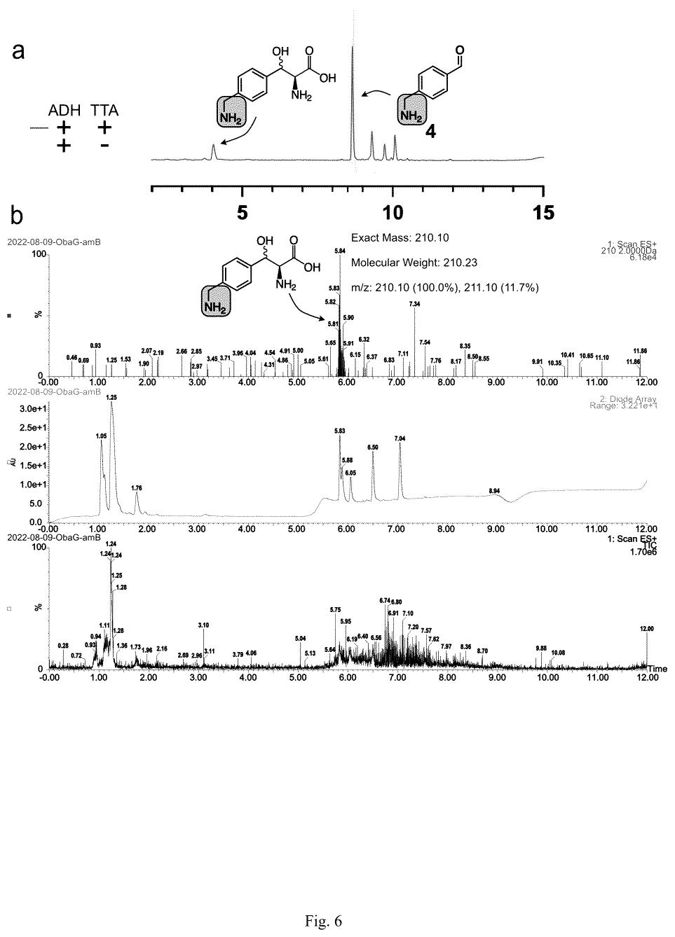

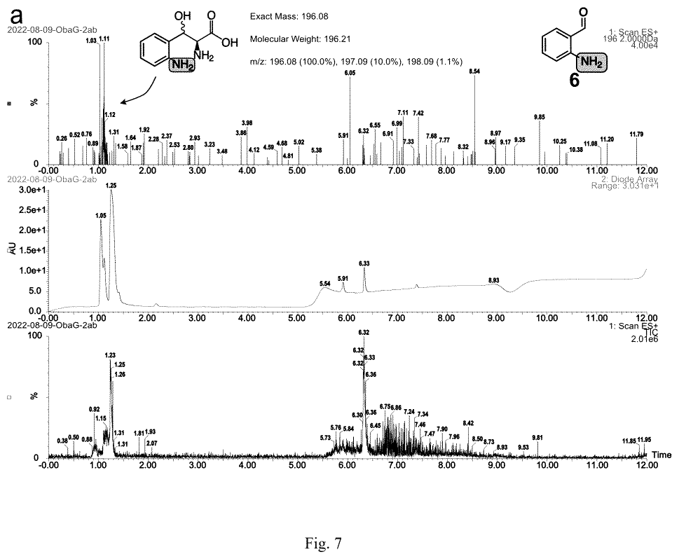

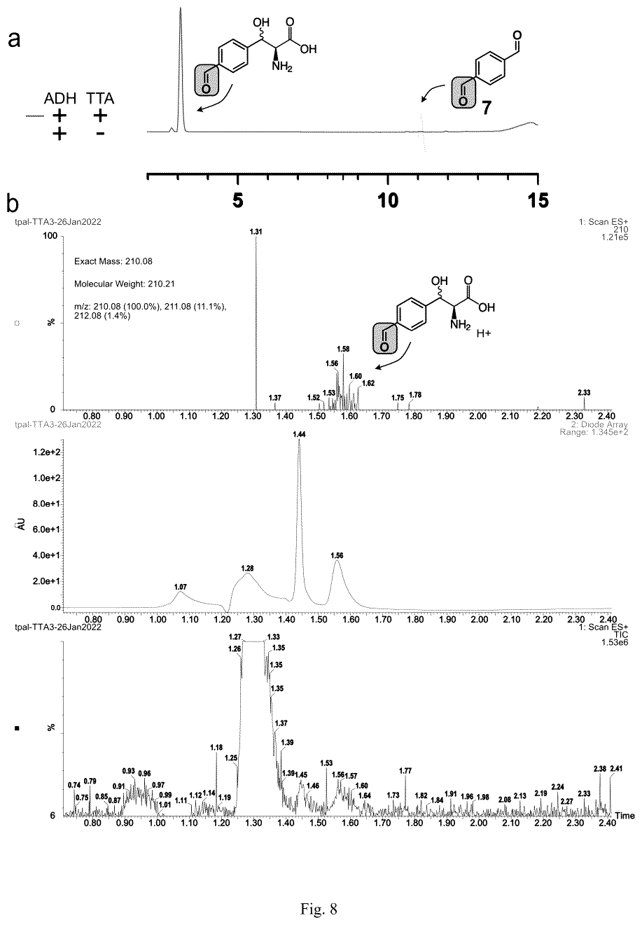

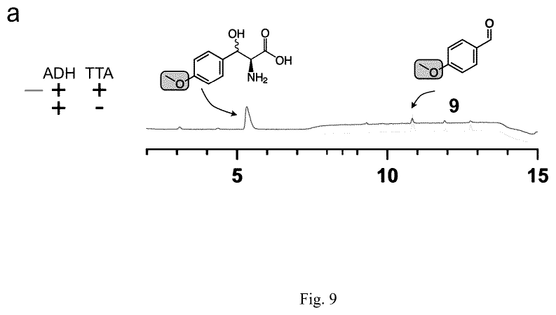

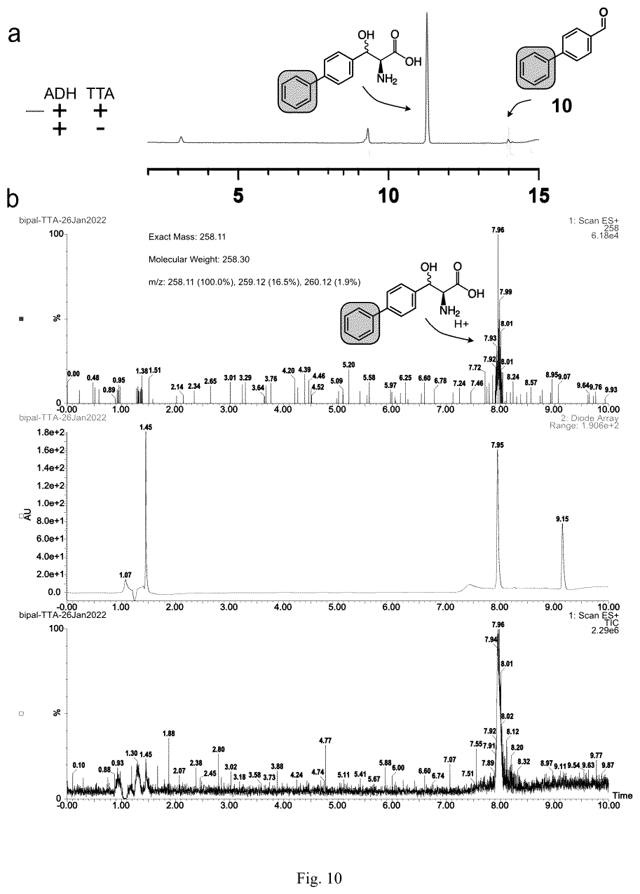

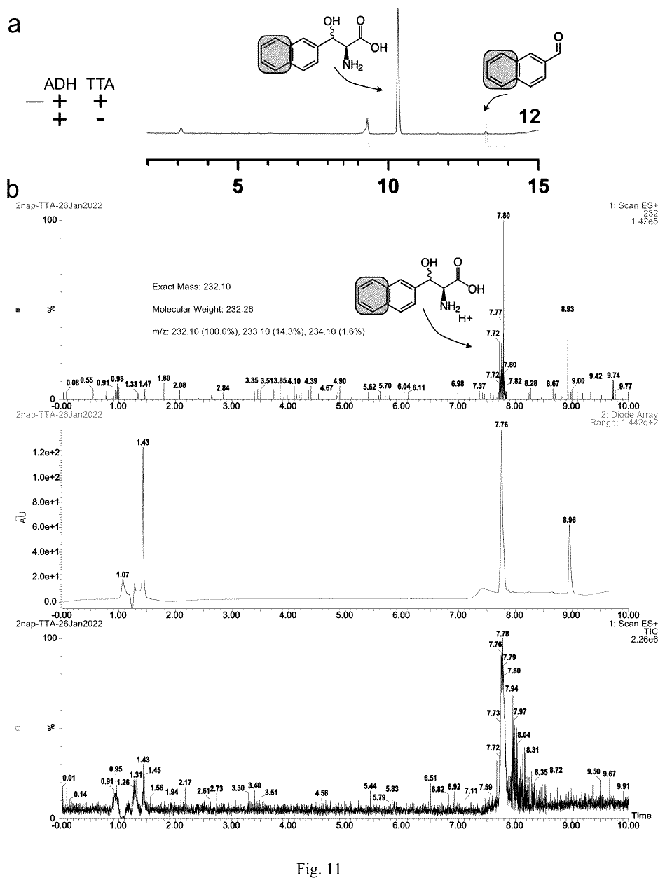

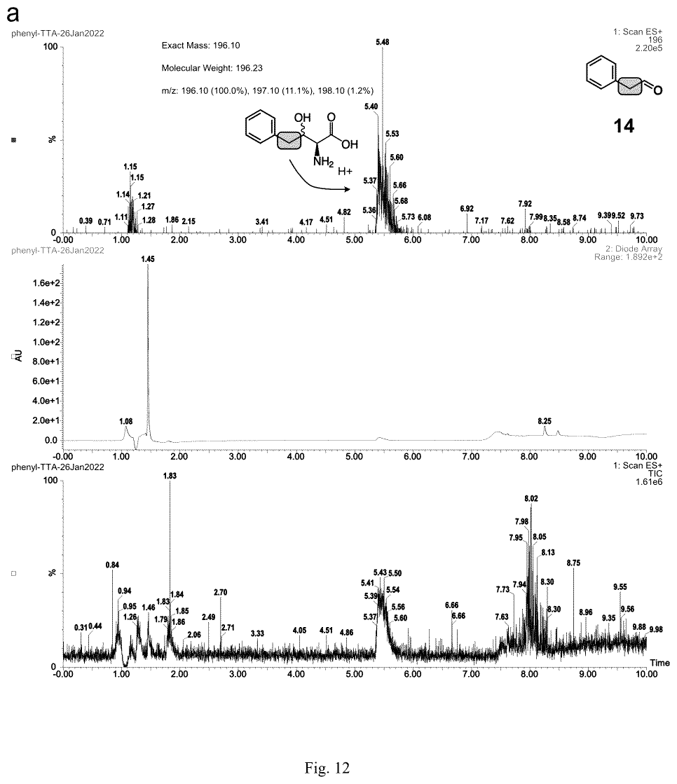

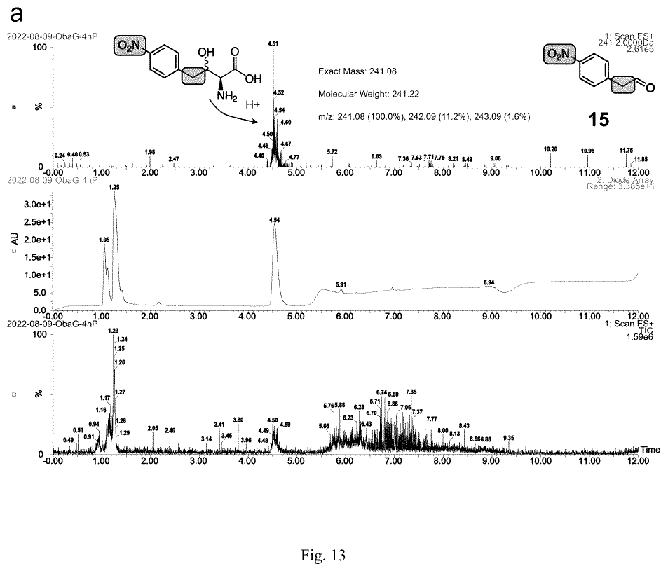

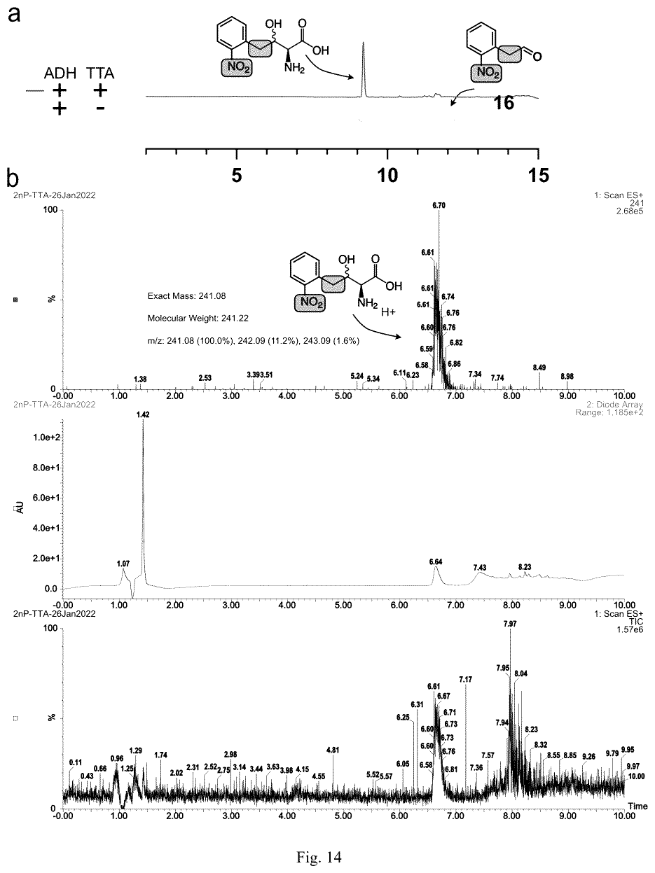

Upon assay validation, we hypothesized that we could rapidly probe the activity of ObiH on diverse aldehydes to expand the potential chemical handles of β-OH-nsAAs. We successfully screened ObiH against 16 unique substrates in a single experiment (FIGS. 2b,c). We validated the activity of ObiH on substrates like the native substrate, 4-nitro-phenylacetaldehyde (15), and 2-nitro-benzaldehyde (3), which ObiH has been reported to exhibit high activity on. Our screen included nine substrates not previously tested with ObiH to our knowledge; activity on seven of these substrates was confirmed with new peak formation via HPLC or LC-MS (FIGS. 3-14). The new substrates include aldehydes that contain amines, conjugatable handles, or larger hydrophobic groups to improve the chemical diversification of β-OH-nsAA products. Our result supported the known general trend that aldehydes containing electron-withdrawing ring substituents are the preferred substrates of ObiH. As expected, the amine-aldehydes were very poor substrates for ObiH, which we hypothesize is because of the strong electron-donating potential of amines. Additionally, one amine-containing substrate (5) absorbed at 340 nm, so it was only tested at low concentrations of 0.25 mM aldehyde (Table 4). Despite this trend, we did observe that there was some activity on aldehydes with moderate electron-donating potential like 4-methoxy-benzaldehyde (9), 4-biphenylcarboxaldehyde (10), and 2-napthalaldehyde (12). Activity on larger, hydrophobic substrates is promising because these substrates can be used to modulate cell permeability for peptides. Additionally, we were excited by the activity of ObiH on terephthalaldehyde (7) and 4-boronobenzaldehyde (13) as those groups can serve as bioconjugatable handles to potentially diversify protein and peptide products. With these results, we hypothesized that the TTA-ADH coupled assay can provide a broad and deep initial lens into functional characterization of this under-explored enzyme class when used under appropriate conditions and with important controls.

### 2.2 Bioprospecting for Novel Putative TTAs

We used bioprospecting as an approach to advance our understanding of the TTA enzyme class and potentially discover a TTA capable of overcoming the limitations of ObiH. Using a protein sequence similarity network (SSN) that was generated with over 800 sequences produced from a BLASTp search of ObiH, LipK, and FTase, we selected 12 additional putative TTAs (FIG. 15a). We selected five putative TTAs from the same cluster as ObiH, all exhibiting >50% sequence identity to ObiH, in addition to seven randomly-selected putative TTAs from clusters with 20%-30% sequence identity to ObiH (FIG. 15b). RaTTA and SNTTA were selected from the cluster containing LipK, DbTTA from the cluster containing FTase, and TmTTA from the cluster containing sequences annotated as SHMTs. Lastly, three TTAs (NoTTA, PbTTA, and KaTTA) were selected from distinct clusters with no characterized enzymes. The broad range of sequence identity of candidate TTAs from 20-80% with respect to ObiH and to each other indicates a broader sampling of the TTA-like sequence space in any one study than past efforts to our knowledge.

Upon selecting our list of candidate TTAs, we proceeded to test heterologous expression of codon-optimized genes in E. coli for purification and in vitro biochemical characterization. Given the reported difficulty of expressing LipK and FTases, we were not surprised to observe little to no expression of the TTAs from the clusters containing FTase and LipK; however, we also observed low expression of TTAs from unexplored clusters, and unexpectedly, two from the cluster containing ObiH. Simple methods for improving protein expression like changing culture temperature were unsuccessful.

Instead, we hypothesized that the appendage of a small solubility tag, the Small Ubiquitin-like Modifier motif (SUMO tag), could improve expression. We were excited to observe that the tag dramatically improved the expression of 11 TTAs (FIG. 15c). To create the option of removing the SUMO tag if it were to impact activity, we cloned a TEV protease site between the SUMO tag and each TTA gene. With the addition of the SUMO tag, we successfully purified nine TTAs for further screening.

### 2.3 Screening and Characterization of Novel TTAs

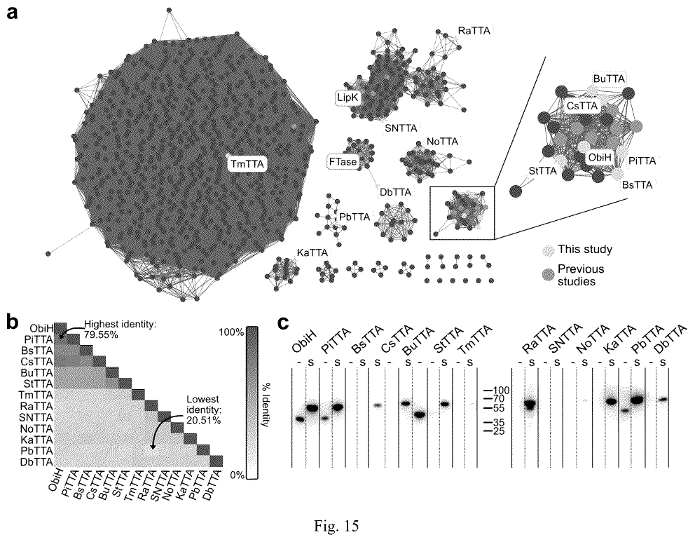

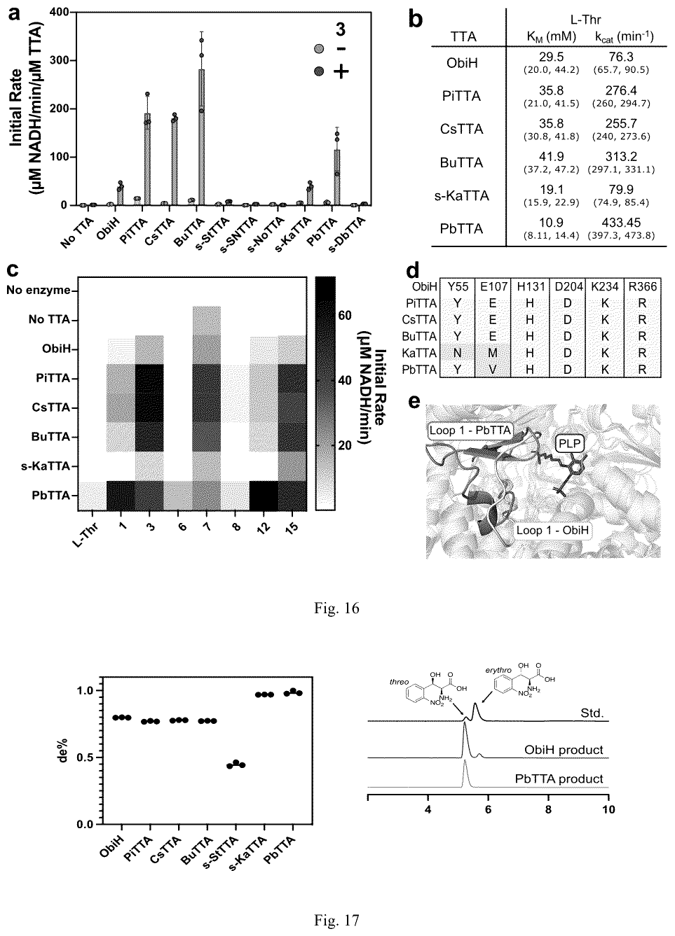

Once purified, we identified the putative TTAs with high activity and further characterized them for their L-Thr affinity and substrate scope. We first screened each purified enzyme using the TTA-ADH coupled assay with 2-nitro-benzaldehyde, 3, the best performing substrate from the screen of ObiH that was not a substrate of the ScADH. We observed that five enzymes (PiTTA, CsTTA, BuTTA, KaTTA, and PbTTA), had activity comparable to or better than ObiH so we characterized these enzymes further (FIG. 16a). We also screened KaTTA with and without the SUMO tag to verify that the tag did not impact activity. With this evidence as well as well-aligned, predicted AlphaFold structures, we assumed the impact of the SUMO tag would be minimal for all TTAs screened and moved forward with additional enzyme characterization. Interestingly, we only observed the vibrant pink color characteristic of ObiH with PiTTA, BuTTA, and KaTTA. All other TTAs had a very faint pink color or no coloration at all.

We next sought to determine the affinity of these enzymes for L-Thr, which we obtained by performing the TTA-ADH coupled assay at different L-Thr concentrations (FIG. 16b). Notably, our assay yielded a lower L-Thr K_{M} for ObiH, 29.5 mM (95% CI: 20.0 mM, 44.2 mM) than the literature value (40.2±3.8 mM). Two differences between our assays were the substrate, phenylacetaldehyde (14) instead of 4-nitrophenylacetylaldehyde (15), and the assay format, ADH coupling rather than a discontinuous HPLC assay. Because a live cellular environment would also contain alcohol dehydrogenases for reduction of acetaldehyde, it is possible that the K_{M} values that we are measuring using the TTA-ADH coupled assay may be more realistic for our envisioned applications. Encouragingly, under these conditions we observed that KaTTA and PbTTA have lower L-Thr K_{M} than ObiH (19.1 mM (95% CI: 15.9 mM, 22.9 mM) and 10.9 mM (95% CI: 8.11 mM, 14.4 mM), respectively) and both had the highest de % for the threo isomer of the β-OH-nsAA using 3 as a substrate (FIG. 17). Interestingly, many of our TTAs such as PiTTA, CsTTA, BuTTA, and PbTTA have higher measured L-Thr k_{cat} values than ObiH using phenylacetaldehyde as the aldehyde substrate (FIG. 16b). Thus, each of the novel characterized enzymes is either faster or has higher L-Thr affinity than ObiH and may prove to be improved alternatives to ObiH depending on the desired application.

Given the broad substrate scope of ObiH, we sought to examine a set of aromatic substrates that would span the spectrum of electronic properties and include some that ObiH exhibits little to no activity on. By providing a set of seven substrates to all six TTAs, we aspired to help elucidate the landscape of specificity within this family while possibly identifying variants that exhibited higher activity or altered specificity (FIG. 16c). We specifically selected substrates with ring substituents with different electron withdrawing properties (1, 3, 6, 7, 8), substituent size (12), and aldehyde chain length (15) to compare the activity of the putative TTAs to ObiH. We were also encouraged by the activity of PbTTA and KaTTA on vanillin and protocatechualdehyde which are substrates that would form products like commercially available therapeutic, Droxidopa (FIG. 18). We observed several interesting behaviors—for example, the TTAs that appeared to have higher k_{cat} values in the ObiH cluster, such as PiTTA and BuTTA, remain relatively selective and are both reported to be a part of biosynthetic gene clusters for obafluorin (Table 5). We were encouraged to find that one of the most active TTAs, PbTTA, also maintains high activity on a diverse array of substrates, originates from a different cluster of the SSN as ObiH, and exhibits low sequence identity (30% identity). This suggests that the TTA enzyme family may be broader than previously thought, with many more active homologs worthy of characterization for the elucidation of natural products or for applications in biocatalysis and synthetic biology.

Given the activity of these distantly related enzymes and their annotation as SHMTs or hypothetical proteins, we wanted to further validate the amino acid substrate specificity of the active enzymes and further screen the inactive TTAs. We performed an in vitro assay over 20 h using 3 as the aldehyde substrate and either L-Thr, Glycine (Gly), or L-Serine (L-Ser) as the candidate amino acid. Since the TTA-ADH coupled assay is specific to L-Thr, we analyzed TTA activity via HPLC with a chemically synthesized β-OH-nsAA standard for the assumed product from 3. We confirmed that the active purified TTAs (PiTTA, CsTTA, BuTTA, KaTTA, and PbTTA) only act with L-Thr with no β-OH-nsAA formation using L-Ser or Gly. Of the inactive enzymes (NoTTA, TmTTA, DbTTA, and StTTA), we observed that StTTA was active with the formation of the β-OH-nsAA product from 3 and L-Thr, suggesting it is too slow to detect using the TTA-ADH coupled assay. NoTTA, TmTTA, and DbTTA yielded no product, which leaves the possibilities that they could be TTAs that do not accept 3 or that they may not be TTAs.

To explore the possibility that DbTTA and TmTTA are TTAs active on other related aldehydes, we sought to examine their activity with L-Thr and aldehyde substrates with different ring substituent position (2), bulkier, hydrophobic chemistry (10), and aldehyde chain length (14) using the TTA-ADH coupled assay. Neither of these proteins appeared to have any TTA activity, nor the reported L-Thr decomposition activity. We did not perform this analysis for NoTTA.

### 2.4 Comparative Sequence Analysis for Newly Reported TTAs

To help shed some light on the potential molecular basis for substrate specificity, we performed a comparative sequence analysis of the active TTAs with a focus on known residues implicated in catalysis (H131, D204, K234) or PLP-stabilization (Y55, E107, and R366) in ObiH, as well as two loop regions that are reported to contribute to substrate specificity. We performed a multiple sequence alignment across the enzymes selected and a series of characterized Type I PLP-dependent enzymes, including LipK from Streptomyces sp. SANK 60405, FTase from Streptomyces cattleya, and SHMT from Methanocaldococcus jannaschii. Many of the active TTAs within the ObiH cluster had the same residues at these sites; however, PbTTA and KaTTA appeared to have modified residues at Y55 and E107 which are reported to perform hydrogen bonding for PLP stabilization (FIG. 16d). This was not surprising as these residues are not conserved across related PLP-dependent enzymes. Further, we evaluated two loop regions from ObiH between Tyr55 and Pro71 (loop 1) as well as Glu355 and His363 (loop 2) that are reported to contribute to substrate specificity given their role in SHMTs as folate binding regions. While loop 1 appears to be composed of different residues across the TTAs screened, PbTTA has a unique 11 amino acid insertion in the equivalent loop 1. We then aligned the published ObiH crystal structure with an AlphaFold prediction for PbTTA and observed a β-sheet within loop 1 of PbTTA whereas loop 1 in ObiH is relatively unstructured (FIG. 16e). Because published MD simulations of ObiH suggest loop 1 is highly flexible, we speculate that the addition of structure in PbTTA may contribute to its broad substrate specificity or low L-Thr K_{M}.

Since this enzyme class is newly discovered, we wanted to explore unique sequence properties of each cluster to determine if there are any distinguishing features across clusters. By aligning all sequences within a cluster to ObiH, we identified that catalytic residues (H131, D204, and K234) are conserved across the clusters containing ObiH, LipK, FTase, KaTTA, and PbTTA. Further, R366 is highly conserved (>90%) for all clusters analyzed. As highlighted for KaTTA and PbTTA, Y55 and E107 are not conserved. The cluster containing KaTTA does not have a conserved residue aligned with Y55. For E107, each cluster appeared to have a different predominant residue in that position. Additionally, given the distinction between the loop 1 of ObiH relative to SHMTs and PbTTA, we wanted to explore the sequence context of this loop region for all the clusters containing TTAs. It appears that this region is a defining characteristic for many of these clusters. Each cluster appears to have on average a different length which may contribute to distinct substrate specificities for each cluster.

### 2.5 In Vivo Production of β-OH-nsAAs

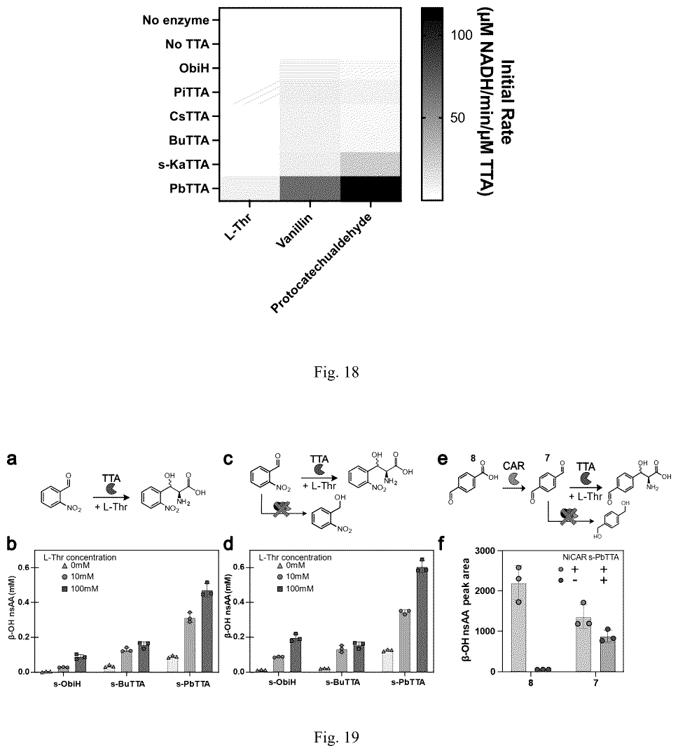

Our last objective was to explore biosynthesis of β-OH-nsAAs in metabolically active cells growing in aerobic conditions given our eventual desire to couple these products to ribosomal and non-ribosomal peptide formation. Production of the targeted β-OH-nsAA using cells that are growing during aerobic fermentation would need to meet three requirements: (1) Soluble expression of TTAs; (2) Affinity towards L-Thr at physiologically relevant concentration; (3) Stability of aromatic aldehyde substrates in the presence of live cells. We hypothesized that the novel TTAs may perform better than ObiH in growing cells because their improved productivity could enable aldehyde utilization prior to aldehyde degradation by the cell. In addition, a higher L-Thr affinity could improve titers achieved in the absence of supplemented L-Thr. Thus, we decided to test the top performing TTAs in live cells and compare titers for different enzymes, specifically ObiH which has the highest expression, PbTTA which has the lowest L-Thr K_{M} and highest k_{cat} but low expression, and BuTTA which has the second highest catalytic rate with high expression. Using the SUMO-tagged constructs, each enzyme was screened in 96-well plate, fermentative conditions in wild-type E. coli MG1655 with 0 mM, 10 mM, and 100 mM L-Thr supplemented and 1 mM 3. We then analyzed titers after 20 h, via HPLC analysis, using the chemically synthesized β-OH-nsAA standard for the assumed product from 3. PbTTA performed the best with the highest titer of 0.47±0.04 mM β-OH-nsAA with 100 mM L-Thr supplemented as well as the highest titer with physiological levels of L-Thr at 0.09±0.01 mM β-OH-nsAA in growing cells (FIGS. 19a,b). Thus, we confirmed production of the β-OH-nsAA in growing cell cultures; however, we hypothesized that we could improve titer by implementing an aldehyde stabilizing strain.

To investigate whether the knockout of genes that encode aldehyde reductases would result in improved yields of β-OH-nsAA, we transformed the plasmid that harbors our TTA expression cassette into another E. coli strain that was engineered to stabilize aromatic aldehydes, the RARE strain. The RARE strain has been shown to stabilize many aromatic aldehydes, including 1, 9, and 12, by eliminating potential reduction pathways. We then repeated the experiment in the RARE strain and once again found that PbTTA produced the highest titer with 0.61±0.04 mM produced with 100 mM L-Thr and 0.13±0.01 mM produced with natural L-Thr levels (FIGS. 19c,d). These improvements with the RARE strain suggest that stabilization of the aldehyde does improve β-OH-nsAA titers, despite observing some reduction of the aldehyde to the corresponding 2-nitro-benzyl alcohol as well as reduction of the nitro-group to an amine. Our study suggests that the E. coli RARE strain transformed to express PbTTA is a promising chassis for β-OH-nsAA production in aerobically grown cells.

Finally, to partially address the toxicity of supplemented aldehydes in fermentative contexts, we investigated whether we could couple a TTA to a carboxylic acid reductase (CAR) to create a steady and low-level supply of aldehydes biosynthesized from carboxylic acid precursors. We coupled PbTTA to a well-studied CAR from Nocardia iowensis to produce a β-OH-nsAA from the corresponding acid in aerobically growing RARE. We performed an initial screen with 2 mM 4-formyl benzoic acid, a proven substrate for NiCAR but not for PbTTA, which would install a conjugatable aldehyde group onto a potential β-OH-nsAA product. We sampled cultures for HPLC analysis 20 h after the addition of the carboxylic acid precursor and observed a peak corresponding to the β-OH-nsAA (FIGS. 19e,f). Additionally, there was greater production of the β-OH-nsAA when starting with the corresponding acid precursor compared to the aldehyde substrate, demonstrating that the addition of the CAR can improve final titers. We are the first to demonstrate the production of this β-OH-nsAA from either the acid or the aldehyde and we were able to produce it in aerobically growing cells. Additionally, the RARE host maintains the aldehyde functional handle of the β-OH-nsAA. The addition of a CAR to this cascade limits the impact of aldehyde toxicity and instability on final product titers and provides the opportunity for future β-OH-nsAA production as a de novo pathway from glucose given the natural abundance of carboxylic acids.

### 2.6 Pathway Development for a Novel Bioconjugatable β-OH-nsAA

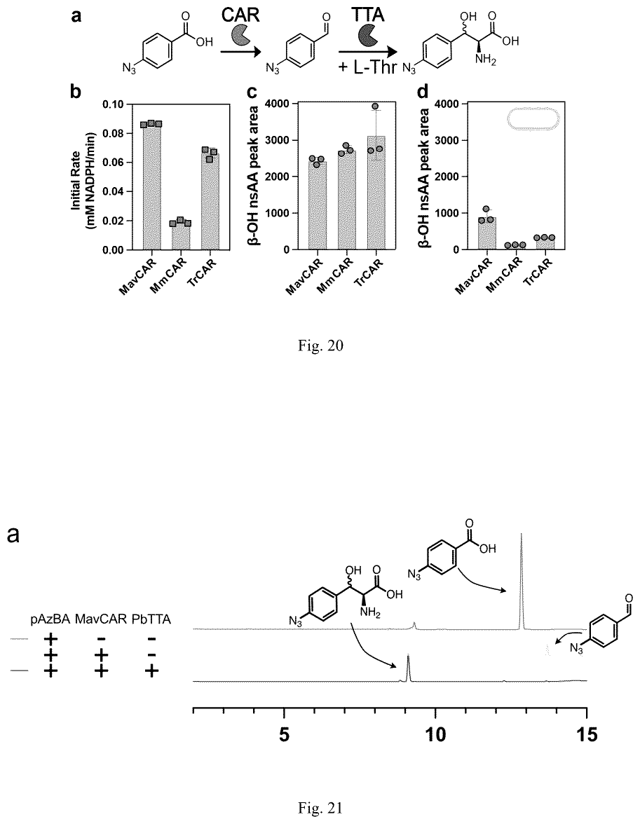

With the promise of the CAR-TTA coupling, we wanted to investigate the generalizability of this pathway to produce a β-OH-nsAA that has a bio-orthogonal conjugation handle. We chose the 4-azido functionality as our target and explored whether it could be made from a 4-azido-benzoic acid precursor. To our knowledge, this precursor would be a substrate never previously tested with any CAR enzyme and its product would be a substrate never tested with any TTA enzyme. Given the prevalence of the azide group as a bio-orthogonal conjugation handle, we selected 4-azido-benzoic acid as the target substrate to produce the corresponding β-OH-nsAA product (FIG. 20a). We first studied a panel of three CARs with a diverse substrate scope and high soluble expression (FIG. 20b). We were excited to observe activity of all the CARs on the acid substrate, so we then coupled the CAR directly to PbTTA in an in vitro assay to identify the β-OH-nsAA (FIG. 20c). The CAR-TTA coupling is valuable because 4-azido-benzaldehyde is expensive ($200 for 250 mg from Toronto Research Chemicals) and likely to be toxic to cells if supplied at high concentrations. The in vitro coupling also successfully produced a β-OH-nsAA product verified as a new peak on the HPLC (FIG. 21). We did observe similar production across all CAR-TTA pairings despite distinct activity of the CARs which suggests that PbTTA may be a limiting step in this cascade. Finally, given the potential to produce novel peptide or protein products in cells, we wanted to confirm the activity of this cascade in growing cells, which was successful for all CAR-TTA pairings with MavCAR producing the highest titer determined by product peak area after 20 h (FIG. 20d). We are the first to produce a β-OH-nsAA that contains an azide functionality from either carboxylic acid or aldehyde precursors, which could be useful for chemical diversification of β-OH-nsAAs, and associated products formed by fermentation using engineered bacteria.

### 3. Discussion

We sought to expand the fundamental understanding of the TTA enzyme class to ultimately develop a platform E. coli strain for fermentative biosynthesis of diverse β-OH-nsAA from supplemented aromatic aldehydes or carboxylic acids. To achieve this, we had to overcome a series of challenges including low protein solubility, low activity on non-ideal substrates, and low L-Thr affinity. We successfully identified a solubility tag that improved expression of 11 of the selected TTAs. We then expressed, purified, and tested nine previously uncharacterized enzymes at the study outset. We successfully identified these TTAs through bioprospecting and rapid analysis of diverse enzymes via an in vitro TTA-ADH coupled assay. Of these novel enzymes, we identified PbTTA, which expresses well in E. coli, can act on a diverse array of substrates, has higher affinity towards L-Thr than ObiH, and has higher catalytic rate when using 14 and L-Thr as substrates. We tested this enzyme in a series of fermentative contexts in an aldehyde-stabilizing strain and coupled it with a CAR to produce β-OH-nsAAs in aerobically grown cells.

Heterologous expression in model bacteria such as E. coli is a well-documented problem for many TTAs, including LipK, and FTase, where ObiH is the exception. The SUMO tag appeared to improve the solubility of many enzymes that share sequence similarity to ObiH, LipK, and FTase, such that some enzymes that were unable to be expressed initially were expressed and purified. Fortunately, the SUMO tag did not appear to impact enzyme activity for the enzymes screened, which agrees with predicted structures. Our findings and further computational predictions suggest that an N-terminal SUMO tag may improve protein expression for similar sequences. Furthermore, our construct design facilitates removal of the tag if needed without impacting enzyme structure.

As a target enzyme for broad biosynthesis, the substrate scope of PsLTTA and ObiH has been studied with several trends suggesting limited activity on aldehydes with electron-donating ring substituents and varying activity based on the position of the ring substitution. We observed similar trends with ObiH; however, we were able to expand the substrate scope to a variety of other substrates including those with some electron-donating properties like 4-methoxy-benzaldehyde, 9. We identified substrates with amine chemistry that appeared to be substrates for ObiH, offering an opportunity for diversification of the potential β-OH-nsAA products. Other chemistries like 4-formyl-boronic acid, 13, and terephthalaldehyde, 7, can act as bioconjugatable and reactive handles for antibiotic and non-ribosomal peptide diversification, as well as for protein engineering applications. Additionally, we wanted to determine if these trends hold for the novel TTAs we identified. Using a selection of aldehydes with different electronic properties, we observed that the TTAs within the ObiH cluster (PiTTA, CsTTA, and BuTTA) maintain the trends observed with ObiH. Further, we observed that PbTTA has a broader substrate scope and maintains high activity on most substrates screened, including 4-azido-benzaldehyde produced from CAR coupling.

The combination of our SSN, our experiments, and our analysis using biosynthetic gene cluster (BGC) discovery tools has revealed that TTAs may be much more versatile in the biosynthesis of natural or unnatural antibiotics than previously understood. The diversity of enzymes that we observed that had TTA activity suggests that there are likely many more natural enzymes capable of performing these aldol condensations. Additionally, the origin of ObiH, LipK, and FTase in natural product synthesis suggests that there may be other natural product syntheses that rely on this chemistry. For example, within the LipK-like enzyme cluster, there are eight published enzymes reported to be a part of several distinct nucleoside antibiotic biosynthetic gene clusters. Of the enzymes we evaluated in our study, RaTTA and SNTTA are a part of predicted spicamycin and muraymycin BGCs, respectively (Table 5). Even with the addition of the SUMO tag, we were only able to purify SNTTA and we observed no TTA activity on aromatic aldehydes. KaTTA, one of the novel active TTAs we identified, is a part of predicted valclavam BGC (Table 5). Upon further analysis, we identified OrfA and an OrfA-like protein described in the literature that are in the same cluster as KaTTA. Interestingly, several enzymes tested and identified to have TTA activity are not a part of any known or characterized BGCs (BuTTA, PbTTA, StTTA). This could provide an opportunity for further exploration of natural products based on the discovery of enzymes with this activity. BuTTA and PbTTA are two such enzymes that warrant further investigation into their genomic context for elucidation of potential natural products.

Finally, we successfully developed an E. coli strain for β-OH-nsAA production by using an aldehyde stabilizing strain and by coupling the TTA with a CAR for β-OH-nsAA production from an acid substrate. There are ample opportunities to explore additional aldehyde and acid substrates, develop new pathways from glucose, and improve accessible L-Thr concentrations with metabolic and genome engineering. The production of diverse β-OH-nsAA in fermentative contexts should also enable formation of complex ribosomally and non-ribosomally translated polypeptides for potential drug discovery. Ultimately, this study brings us a step closer to a platform E. coli strain for production of diverse β-OH-nsAAs in fermentative contexts.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

TABLE 1
Strains and Plasmids

| Number | Name | Relevant genotype | Source |
|---|---|---|---|
| E. coli strains | | | |
| | DH5α | F− Φ80lacZΔM15 Δ(lacZYA-argF) U169 | NEB |
| | | recA1 endA1 hsdR17 (rK−, mK+) phoA | |
| | | supE44 λ− thi-1 gyrA96 relA1 | |
| | MG1655 | F− λ− ilvG− rfb-50 rph-1 | ATCC 700926 |
| | MG1655 (DE3) | F− λ− ilvG− rfb-50 rph-1 (λ DE3) | Previous study |
| | | λ DE3 = λ sBamHIo ΔEcoRI-B | (Kunjapur et al. |
| | | int::(lacI::PlacUV5::T7 gene1) i21 | JACS, 2014) |
| | | Δnin5 | |
| | RARE | MG1655(DE3) ΔdkgB ΔyeaE Δ(yqhC- | Previous study |
| | | dkgA) ΔyahK ΔyjgB | (Kunjapur et al. |
| | | | JACS, 2014) |
| | BL21 (DE3) | fhuA2 [Ion] ompT gal (λ DE3) [dcm] | NEB |
| | | ΔhsdS | |
| 1-13 | MAJ01-MAJ13 | DH5α harboring TTA expression | This study |
| | | plasmids P1-P13 | |
| 14-26 | MAJ14-MAJ26 | BL21 (DE3) harboring TTA expression | This study |
| | | plasmids P1-P13 | |
| 27-39 | MAJ27-MAJ39 | MG1655 (DE3) harboring TTA | This study |
| | | expression plasmids P1-P13 | |
| 40-52 | MAJ40-MAJ52 | DH5α harboring SUMO-tagged TTA | This study |
| | | expression plasmids P14-P26 | |
| 53-65 | MAJ53-MAJ65 | BL21 (DE3) harboring SUMO-tagged | This study |
| | | TTA expression plasmids P14-P26 | |
| 66-78 | MAJ66-MAJ78 | MG1655 (DE3) harboring SUMO- | This study |
| | | tagged TTA expression plasmids P14- | |
| | | P26 | |
| 79-91 | MAJ79-MAJ91 | RARE harboring SUMO-tagged TTA | This study |
| | | expression plasmids P14-P26 | |
| 92 | MAJ92 | DH5α harboring TTA expression | This study |
| | | plasmid P27 | |
| 93-96 | MAJ93-96 | DH5α harboring CAR expression | Previous studies |
| | | plasmids P28-P31 | (Gopal et al. |
| | | | biorxiv, 2022 |
| | | | and Kunjapur et |
| | | | al. JACS, 2014) |
| 97 | MAJ97 | RARE harboring pACYC-niCAR-sfp | This study |
| | | (P28) and pZE-SUMO-PbTTA(P25) | |
| 98 | MAJ98 | RARE harboring pACYC-SUMO-PbTTA | This study |
| | | (P27) | |
| 99 | MAJ99 | RARE harboring pZE-mavCAR-sfp | This study |
| | | (P29) and pACYC-SUMO-PbTTA (P27) | |
| 100 | MAJ100 | RARE harboring pZE-mmCAR-sfp | This study |
| | | (P30) and pACYC-SUMO-PbTTA (P27) | |
| 101 | MAJ101 | RARE harboring pZE-trCAR-sfp (P31) | This study |
| | | and pACYC-SUMO-PbTTA (P27) | |
| 102-105 | MAJ102-105 | BL21 (DE3) harboring CAR expression | Previous study |
| | | plasmids P28-31 | (Gopal et al. |
| | | | biorxiv, 2022) |
| 106-109 | MAJ106-109 | DH5α harboring ADH expression | This study |
| | | plasmids P32-P35 | |
| 110-113 | MAJ110-113 | BL21 (DE3) harboring ADH expression | This study |
| | | plasmids P32-35 | |
| 114 | MAJ114 | DH5a harboring PTDH expression | Previous study |
| | | plasmids P36. pET15b-17X-PTDH was | (Yang et al. |
| | | a gift from Wilfred van der Donk | JACS, 2015) |
| | | (Addgene plasmid # 166786; | |
| | | http://n2t.net/addgene: 166786; | |
| | | RRID: Addgene_166786). | |
| 115 | MAJ115 | BL21 (DE3) harboring PTDH | This study |
| | | expression plasmid P36 | |
| Plasmids | | | |
| P1 | pZE-ObiH | ColE1 ori, Kan^{R}, TetR, Tet promoter | This study |
| | | with a codon optimized obiH gene | |
| | | bearing an N-terminal hexahistidine | |
| | | tag. | |
| P2 | PZE-PITTA | ColE1 ori, Kan^{R}, TetR, Tet promoter | This study |
| | | with a codon optimized piTTA gene | |
| | | bearing an N-terminal hexahistidine | |
| | | tag. | |
| P3 | pZE-BsTTA | ColE1 ori, Kan^{R}, TetR, Tet promoter | This study |
| | | with a codon optimized bsTTA gene | |
| | | bearing an N-terminal hexahistidine | |
| | | tag. | |
| P4 | pZE-CsTTA | ColE1 ori, Kan^{R}, TetR, Tet promoter | This study |
| | | with a codon optimized csTTA gene | |
| | | bearing an N-terminal hexahistidine | |
| | | tag. | |
| P5 | pZE-BuTTA | ColE1 ori, Kan^{R}, TetR, Tet promoter | This study |
| | | with a codon optimized buTTA2 gene | |
| | | bearing an N-terminal hexahistidine | |
| | | tag. | |
| P6 | pZE-StTTA | ColE1 ori, Kan^{R}, TetR, Tet promoter | This study |
| | | with a codon optimized stTTA gene | |
| | | bearing an N-terminal hexahistidine | |
| | | tag. | |
| P7 | pZE-TmTTA | ColE1 ori, Kan^{R}, TetR, Tet promoter | This study |
| | | with a codon optimized tmTTA gene | |
| | | bearing an N-terminal hexahistidine | |
| | | tag. | |
| P8 | pZE-RaTTA | ColE1 ori, Kan^{R}, TetR, Tet promoter | This study |
| | | with a codon optimized raTTA gene | |
| | | bearing an N-terminal hexahistidine | |
| | | tag. | |
| P9 | pZE-SNTTA | ColE1 ori, Kan^{R}, TetR, Tet promoter | This study |
| | | with a codon optimized snTTA gene | |
| | | bearing an N-terminal hexahistidine | |
| | | tag. | |
| P10 | pZE-NOTTA | ColE1 ori, Kan^{R}, TetR, Tet promoter | This study |
| | | with a codon optimized noTTA gene | |
| | | bearing an N-terminal hexahistidine | |
| | | tag. | |
| P11 | pZE-KaTTA | ColE1 ori, Kan^{R}, TetR, Tet promoter | This study |
| | | with a codon optimized kaTTA gene | |
| | | bearing an N-terminal hexahistidine | |
| | | tag. | |
| P12 | pZE-PbTTA | ColE1 ori, Kan^{R}, TetR, Tet promoter | This study |
| | | with a codon optimized pbTTA gene | |
| | | bearing an N-terminal hexahistidine | |
| | | tag. | |
| P13 | pZE-DbTTA | ColE1 ori, Kan^{R}, TetR, Tet promoter | This study |
| | | with a codon optimized dbTTA gene | |
| | | bearing an N-terminal hexahistidine | |
| | | tag. | |
| P14 | pZE-SUMO- | ColE1 ori, Kan^{R}, TetR, Tet promoter | This study |
| | ObiH | with a codon optimized obiH gene | |
| | | bearing an N-terminal hexahistidine | |
| | | tag. | |
| P15 | pZE-SUMO- | ColE1 ori, Kan^{R}, TetR, Tet promoter | This study |
| | PITTA | with a codon optimized piTTA gene | |
| | | bearing an N-terminal hexahistidine | |
| | | tag followed by a SUMO tag and a TEV | |
| | | protease cleavage site. | |
| P16 | pZE-SUMO- | ColE1 ori, Kan^{R}, TetR, Tet promoter | This study |
| | BsTTA | with a codon optimized bsTTA gene | |
| | | bearing an N-terminal hexahistidine | |
| | | tag followed by a SUMO tag and a TEV | |
| | | protease cleavage site. | |
| P17 | pZE-SUMO- | ColE1 ori, Kan^{R}, TetR, Tet promoter | This study |
| | CsTTA | with a codon optimized csTTA gene | |
| | | bearing an N-terminal hexahistidine | |
| | | tag followed by a SUMO tag and a TEV | |
| | | protease cleavage site. | |
| P18 | pZE-SUMO- | ColE1 ori, Kan^{R}, TetR, Tet promoter | This study |
| | BuTTA | with a codon optimized buTTA2 gene | |
| | | bearing an N-terminal hexahistidine | |
| | | tag followed by a SUMO tag and a TEV | |
| | | protease cleavage site. | |
| P19 | pZE-SUMO- | ColE1 ori, Kan^{R}, TetR, Tet promoter | This study |
| | StTTA | with a codon optimized stTTA gene | |
| | | bearing an N-terminal hexahistidine | |
| | | tag followed by a SUMO tag and a TEV | |
| | | protease cleavage site. | |
| P20 | pZE-SUMO- | ColE1 ori, Kan^{R}, TetR, Tet promoter | This study |
| | TmTTA | with a codon optimized tmTTA gene | |
| | | bearing an N-terminal hexahistidine | |
| | | tag followed by a SUMO tag and a TEV | |
| | | protease cleavage site. | |
| P21 | pZE-SUMO- | ColE1 ori, Kan^{R}, TetR, Tet promoter | This study |
| | RaTTA | with a codon optimized raTTA gene | |
| | | bearing an N-terminal hexahistidine | |
| | | tag followed by a SUMO tag and a TEV | |
| | | protease cleavage site. | |
| P22 | pZE-SUMO- | ColE1 ori, Kan^{R}, TetR, Tet promoter | This study |
| | SNTTA | with a codon optimized snTTA gene | |
| | | bearing an N-terminal hexahistidine | |
| | | tag followed by a SUMO tag and a TEV | |
| | | protease cleavage site. | |
| P23 | pZE-SUMO- | ColE1 ori, Kan^{R}, TetR, Tet promoter | This study |
| | NOTTA | with a codon optimized noTTA gene | |
| | | bearing an N-terminal hexahistidine | |
| | | tag followed by a SUMO tag and a TEV | |
| | | protease cleavage site. | |
| P24 | pZE-SUMO- | ColE1 ori, Kan^{R}, TetR, Tet promoter | This study |
| | KaTTA | with a codon optimized kaTTA gene | |
| | | bearing an N-terminal hexahistidine | |
| | | tag followed by a SUMO tag and a TEV | |
| | | protease cleavage site. | |
| P25 | pZE-SUMO- | ColE1 ori, Kan^{R}, TetR, Tet promoter | This study |
| | PbTTA | with a codon optimized pbTTA gene | |
| | | bearing an N-terminal hexahistidine | |
| | | tag followed by a SUMO tag and a TEV | |
| | | protease cleavage site. | |
| P26 | pZE-SUMO- | ColE1 ori, Kan^{R}, TetR, Tet promoter | This study |
| | DbTTA | with a codon optimized dbTTA gene | |
| | | bearing an N-terminal hexahistidine | |
| | | tag followed by a SUMO tag and a TEV | |
| | | protease cleavage site. | |
| P27 | pACYC-SUMO- | P15A ori, Cm^{R}, lacI, T7lac with codon | This study |
| | PbTTA | optimized SUMO-tagged PbTTA | |
| P28 | pACYC-niCAR- | pACYCDuet-1 harboring a codon | Previous study |
| | sfp | optimized carboxylic acid reductase | (Kunjapur et al. |
| | | from Norcardia iowensis (niCAR) and a | JACS, 2014) |
| | | codon optimized phosphopantetheinyl | |
| | | transferase from Bacillus subtilis (sfp). | |
| | | P15A ori, Cm^{R}, lacI, T7lac | |
| P29 | pZE-mavCAR- | ColE1 Ori, Kan^{R}, TetR, Tet promoter | Previous study |
| | sfp | with a codon optimized carboxylic acid | (Gopal et al. |
| | | reductase from Mycobacterium avium | biorxiv, 2022) |
| | | (mavCAR) and a codon optimized | |
| | | phosphopantetheinyl transferase from | |
| | | Bacillus subtilis (sfp). | |
| P30 | pZE-mmCAR- | ColE1 Ori, Kan^{R}, TetR, Tet promoter | Previous study |
| | sfp | with a codon optimized carboxylic acid | (Gopal et al. |
| | | reductase from Mycobacterium | biorxiv, 2022) |
| | | marinum (mmCAR) and a codon | |
| | | optimized phosphopantetheinyl | |
| | | transferase from Bacillus subtilis (sfp). | |
| P31 | pZE-trCAR-sfp | ColE1 Ori, Kan^{R}, TetR, Tet promoter | Previous study |
| | | with a codon optimized carboxylic acid | (Gopal et al. |
| | | reductase from Trichoderma reesei | biorxiv, 2022) |
| | | (trCAR) and a codon optimized | |
| | | phosphopantetheinyl transferase from | |
| | | Bacillus subtilis (sfp). | |
| P32 | pZE-eutG- | ColE1 Ori, Kan^{R}, TetR, Tet promoter | This study |
| | Ctermhis | with an alcohol dehydrogenase (eutG) | |
| | | from Escherichia coli. | |
| P33 | pZE-adhP- | ColE1 Ori, Kan^{R}, TetR, Tet promoter | This study |
| | Ctermhis | with an alcohol dehydrogenase (adhP) | |
| | | from Escherichia coli. | |
| P34 | pZE-adhE- | ColE1 Ori, Kan^{R}, TetR, Tet promoter | This study |
| | Ctermhis | with an alcohol dehydrogenase (adhE) | |
| | | from Escherichia coli. | |
| P35 | pZE-fucO- | ColE1 Ori, Kan^{R}, TetR, Tet promoter | This study |
| | Ctermhis | with an alcohol dehydrogenase (fucO) | |
| | | from Escherichia coli. | |
| P36 | pET15b-17X- | pBR322 ori, AmpR, LacI, T7 promoter | Previous study |
| | PTDH | with a phosphite dehydrogenase | (Yang et al. |
| | | (PTDH) from Pseudomonas stutzeri | JACS, 2015) |
| | | containg the following mutations for | |
| | | activity: A196R, T201S, A328T, | |
| | | E352N, C356D. | |

TABLE 2
Oligonucleotides

| | | SEQ |
|---|---|---|
| | | ID |
| Oligo Name | Sequence | NO |
| pZE backbone | CTTGATGGGGGATCCCATGGTA | 56 |
| FWD | | |
| pZE backbone | GTGGTGATGATGGTGATGGCTGCTGCCCATGGTACCTTTCTC | 57 |
| REV | CTCTTTAATGAATTCG | |
| StTTA REV | CCATGGGATCCCCCATCAAGTTAACGAAAGACCTCACCCAAC | 58 |
| | A | |
| BuTTA REV | CCATGGGATCCCCCATCAAGTTAAGCGATTACTTCCTCCATCA | 59 |
| | A | |
| PiTTA REV | CCATGGGATCCCCCATCAAGTTAGCGTTGAATTCCACGCTC | 60 |
| ObiH-REV | CCATGGGATCCCCCATCAAGTTAACGTTGGGCTCCTTGG | 61 |
| BsTTA REV | CCATGGGATCCCCCATCAAGTTAACGCATCACGCCTTGG | 62 |
| CsTTA REV | CCATGGGATCCCCCATCAAGTTAGCGTAACGCCTCCCCAATA | 63 |
| StTTA FWD | GCCATCACCATCATCACCACATGGGAGTTTGGGCAGGC | 64 |
| BuTTA FWD | GCCATCACCATCATCACCACATGATGACGGACTTCGCA | 65 |
| PiTTA FWD | GCCATCACCATCATCACCACATGAAACAAGACGAATCGAATG | 66 |
| ObiH-FWD | GCCATCACCATCATCACCACATGTCCAATGTCAAGCAACA | 67 |
| BsTTA FWD | GCCATCACCATCATCACCACATGAAACAGGAACCTACGGG | 68 |
| CsTTA FWD | GCCATCACCATCATCACCACATGACGCGCACGACCC | 69 |
| BsTTA SEQ | GTGCCCGAACATTCAGAG | 70 |
| StTTA SEQ | GCGTATATTGCGTTCCG | 71 |
| BuTTA SEQ | ACCATCCTGCGATGAAG | 72 |
| PiTTA SEQ | AAAGGGGTTTATTGCGTTCA | 73 |
| CsTTA SEQ | GCGGGTCATTTACATCGT | 74 |
| PiTTA SUMO FWD | GAAAATCTGTATTTTCAGGGCAAACAAGACGAATCGAATGTT | 75 |
| | G | |
| TEV SUMO REV | GCCCTGAAAATACAGATTTTCTG | 76 |
| BsTTA SUMO FWD | GAAAATCTGTATTTTCAGGGCAAACAGGAACCTACGGGC | 77 |
| StTTA SUMO FWD | AAAATCTGTATTTTCAGGGCGGAGTTTGGGCAGGCGAC | 78 |
| pZE split REV V1 | CCTGGTATCTTTATAGTCCTGTCGG | 79 |
| CsTTA SUMO FWD | AAAATCTGTATTTTCAGGGCACGCGCACGACCCCCCAG | 80 |
| pZE split REV V2 | GGGAAACGCCTGGTATCTTTATAGTCCTGTCGG | 81 |
| ObiH SUMO FWD | AAAATCTGTATTTTCAGGGCTCCAATGTCAAGCAACAGAC | 82 |
| PbTTA SUMO FWD | AAAATCTGTATTTTCAGGGCGAAACCTCCCTGAAGGATTTTG | 83 |
| BuTTA SUMO FWD | AAAATCTGTATTTTCAGGGCACGGACTTCGCACAGGC | 84 |
| BuTTA SUMO REV | ACGCCTGGTATCTTTATAGTCCTGTC | 85 |
| RaTTA gene fwd | GCCATCACCATCATCACCACATGTTGGAAATTGTGGGGG | 86 |
| RaTTA gene rev | CCATGGGATCCCCCATCAAGTTAACGATAAAGCCACGCAG | 87 |
| pZE bbone fwd | CTTGATGGGGGATCCCATG | 88 |
| pZE bbone rev | GTGGTGATGATGGTGATGG | 89 |
| TmTTA gene fwd | GCCATCACCATCATCACCACATGCGCGAGGAAGAAGC | 90 |
| TmTTA gene rev | CCATGGGATCCCCCATCAAGTTACAGTAACGGAAGACAAGGG | 91 |
| SnTTA gene fwd | GCCATCACCATCATCACCACATGACATCAAGCGACGATTG | 92 |
| SnTTA gene rev | CCATGGGATCCCCCATCAAGTTACCCATGAAAAAGTCCCG | 93 |
| NoTTA gene fwd | GCCATCACCATCATCACCACATGAATACGTTCGATATCTTAGA | 94 |
| | AC | |
| NoTTA gene rev | CCATGGGATCCCCCATCAAGTTATGCGACTGATACCTCC | 95 |
| PbTTA gene fwd | GCCATCACCATCATCACCACATGGAAACCTCCCTGAAGG | 96 |
| PbTTA gene rev | CCATGGGATCCCCCATCAAGTTAGAATAACTTCTCGTAGATCT | 97 |
| | CG | |
| DbTTA gene fwd | GCCATCACCATCATCACCACTTGACGAATAATCGCGAGC | 98 |
| DbTTA gene rev | CCATGGGATCCCCCATCAAGTTAAGAGGCATAGACCGCC | 99 |
| KaTTA gene fwd | GCCATCACCATCATCACCACATGGATGTGTTGGCTGC | 100 |
| KaTTA gene rev | CCATGGGATCCCCCATCAAGTTAGGCTACTGCCAAGGG | 101 |
| SUMO tag fwd | ATGTCCCTGCAGGACTC | 102 |
| SUMO tag rev | GCCCTGAAAATACAGATTTTCTGAACCTCCACCTCCCGACCCA | 103 |
| | CCACCGCCGCCACCAATCTGTTCGC | |
| pZE-SWNB bbone | TCCGAGTCCTGCAGGGACATGTGGTGATGATGGTGATGG | 104 |
| rev | | |
| pZE-TmTTA | AAAATCTGTATTTTCAGGGCATGCGCGAGGAAGAAGC | 105 |
| bbone fwd | | |
| pZE-RaTTA | AAAATCTGTATTTTCAGGGCATGTTGGAAATTGTGGGGG | 106 |
| bbone fwd | | |
| pZE-SnTTA | AAAATCTGTATTTTCAGGGCATGACATCAAGCGACGATTG | 107 |
| bbone fwd | | |
| pZE-NoTTA | AAAATCTGTATTTTCAGGGCATGAATACGTTCGATATCTTAGA | 108 |
| bbone fwd | AC | |
| pZE-TmTTA | TCCGAGTCCTGCAGGGACATGTGGTGATGATGGTGATGGC | 109 |
| bbone rev | | |
| pZE-DbTTA | AAAATCTGTATTTTCAGGGCTTGACGAATAATCGCGAGC | 110 |
| bbone fwd | | |
| pZE-KaTTA | AAAATCTGTATTTTCAGGGCATGGATGTGTTGGCTGC | 111 |
| bbone fwd | | |
| pACYC bbone fwd | AAGCTTGATGGGGGATC | 112 |
| pACYC bbone rev | GGTATATCTCCTTATTAAAGTTAAAC | 113 |
| pACYC SUMO- | CTTTAATAAGGAGATATACCATGGGCAGCAGCCATCA | 114 |
| PbTTA12 ins fwd | | |
| pACYC SUMO- | GGATCCCCCATCAAGCTTTTAGAATAACTTCTCGTAGATCTCG | 115 |
| PbTTA12 ins rev | T | |

TABLE 3
DNA G-Blocks/Twist Gene Fragments⁺

| | Protein | | SEQ |
|---|---|---|---|
| | Accession | | ID |
| Name | No. | Sequence | NO |
| ObiH | ARJ35753.1 | ATGTCCAATGTCAAGCAACAGACAGCTCAGATCGTGGATTG | 44 |
| | | GTTATCAAGCACTTTAGGTAAAGACCATCAGTATCGTGAAG | |
| | | ATAGCTTGAGTCTTACAGCGAACGAGAACTATCCGTCAGCG | |
| | | TTGGTACGTTTGACGTCGGGCTCGACCGCAGGGGCGTTTT | |
| | | ATCACTGTAGTTTCCCCTTTGAGGTACCTGCCGGGGAATGG | |
| | | CACTTCCCGGAGCCAGGGCATATGAATGCCATCGCAGACC | |
| | | AGGTACGTGATCTTGGGAAAACACTGATCGGAGCACAGGC | |
| | | GTTTGACTGGCGCCCAAACGGCGGCTCTACAGCAGAACAG | |
| | | GCGTTGATGTTAGCGGCGTGCAAGCCCGGGGAAGGATTTG | |
| | | TCCATTTCGCACACCGCGACGGAGGCCATTTTGCGCTTGAA | |
| | | TCACTGGCGCAGAAAATGGGAATTGAAATTTTCCACTTGCC | |
| | | AGTTAACCCCACGAGTTTGCTTATTGATGTGGCGAAATTGG | |
| | | ATGAAATGGTCCGCCGCAATCCGCACATCCGTATTGTAATT | |
| | | CTGGACCAGTCCTTTAAGCTTCGCTGGCAGCCGTTGGCGG | |
| | | AAATTCGTTCCGTACTGCCGGATTCGTGTACTTTGACGTAC | |
| | | GACATGAGTCACGATGGAGGTTTGATTATGGGTGGCGTTTT | |
| | | CGATTCGCCTTTAAGTTGCGGAGCAGACATCGTACACGGAA | |
| | | ACACACATAAGACGATCCCTGGTCCACAGAAAGGGTACATC | |
| | | GGATTTAAGAGTGCTCAACACCCGCTGTTAGTGGATACCAG | |
| | | CCTTTGGGTATGCCCTCACCTGCAATCCAACTGCCATGCGG | |
| | | AACAGCTGCCGCCAATGTGGGTAGCATTCAAAGAAATGGA | |
| | | ACTGTTCGGGCGTGATTACGCGGCCCAAATTGTGTCAAATG | |
| | | CTAAGACCTTGGCACGTCACTTGCACGAGTTAGGATTAGAC | |
| | | GTTACGGGGGAGAGCTTTGGGTTTACCCAGACTCACCAGG | |
| | | TACACTTCGCTGTAGGCGACTTACAAAAAGCCTTGGATTTA | |
| | | TGTGTTAATTCACTTCACGCAGGGGGCATCCGTAGCACGAA | |
| | | TATCGAGATTCCCGGAAAACCAGGGGTGCATGGTATTCGTT | |
| | | TGGGTGTGCAAGCGATGACTCGCCGTGGCATGAAGGAAAA | |
| | | GGATTTCGAGGTGGTAGCTCGTTTCATTGCGGATCTTTACT | |
| | | TCAAGAAAACTGAGCCAGCGAAAGTTGCTCAGCAGATTAAG | |
| | | GAATTTTTGCAGGCGTTCCCATTAGCGCCTCTGGCATATTC | |
| | | TTTTGATAATTATTTAGACGAGGAGTTATTGGCTGCGGTGT | |
| | | ACCAAGGAGCCCAACGTTAA | |
| PiTTA | WP_095149064.1 | ATGAAACAAGACGAATCGAATGTTGGTCCTGTCATTGACTG | 45 |
| | | GCTGGCTCAGACCCTTGGACAGGACTACAAGTACCGCCAG | |
| | | GACACACTTTCACTTACAGCTAACGAAAACTACCCTTCAGA | |
| | | GCTTGTTCGTCTGACCAGCGGCTCTACAGCCGGGGCATTTT | |
| | | ATCACTGCTCTTTTCCGTTCCCCGTTCCTCTTGGAGAATGG | |
| | | CATTTCCCAGAGCCAGGACAAATGAACGAGATCGCCGATG | |
| | | ATCTGCGCGGTTTGGCCAAACGTATGATGGGTGCGCAGGC | |
| | | ATTCGATTGGCGCCCTAATGGTGGGAGCCCGGCTGAACAG | |
| | | GCCTTGATGTTAGCGGCTTGTAAACAAGGTGAAGGTTTTGT | |
| | | ACACTTTGCACATCGCGATGGGGGGCATTTTGCTTTAGAGC | |
| | | AATTGGCGACAAAAATGGGTATTGAGATTTTCCATTTACCT | |
| | | GTGGATCCGCAAAGTCTGCTTATTGACGTTGCTAAGCTTGA | |
| | | TGACATGGTCCGCCGTAACCCTCACATCCGTATCGTAATTC | |
| | | TTGATCAATCCTTCAAACTTCGTTGGCAGCCGTTAGCCGAG | |
| | | ATTCGTGCAATCCTTCCCGATTCATGCACTTTAACTTATGAT | |
| | | ATGTCTCATGATGGGGGCCTTATTCTGGGTGGGGTCTTCGA | |
| | | TAGCCCATTGGCGTGCGGTGCGGATATCGCTCACGGCAAT | |
| | | ACTCACAAGACTATTCCGGGGCCTCAAAAGGGGTTTATTGC | |
| | | GTTCAAGAGCGCTCAGCACCCCCTGTTGGTGGAAACCAGT | |
| | | CTTTGGGTATGTCCACACTTACAGAGTAACTGTCACGCCGA | |
| | | ACTTTTACCCTCTATGTGGGCCGCATTCAAGGAGATGGAAG | |
| | | CTTTTGGCCCCGCCTATGCCCACCAGATGGTGCGCAATGCT | |
| | | AAGGCGTTGGCCAACCAACTTCACGAGCTTGGTTTAAATGT | |
| | | TTCGGGAGAGTCTTTTGGATTTACAGAGACGCACCAGGTGC | |
| | | ATTTCGCCGTAGGAGATTTACAACAGGCGTTGAGTATGTGC | |
| | | GTGGACTCGTTACACGCGGGCGGAATCCGCTCGACTAACA | |
| | | TCGAGATCCCGGGAAAGCCCGGGATGCACGGGATCCGCTT | |
| | | GGGGGTACAGGCCATGACCCGCCGCGGTATGAAAGAGGAT | |
| | | GACTTTCGTCGCGTCGCTGGCCTTATCGCTGACCTTTACTT | |
| | | CAAGCGTACCGAACCTGCACGTGTTGCTTCAAAGGTGAAG | |
| | | GAGTTATTGGGCGATTTTCCACTTGCCCCTCTGGCCTACTC | |
| | | GTTCGATCAACAAATCGACGAGTCTCGCCGCCGTTTGCTTG | |
| | | AGCGTGGAATTCAACGCTAA | |
| BsTTA | WP_060149112.1 | ATGAAACAGGAACCTACGGGCGCCTTCGAGGTTGCCACGG | 46 |
| | | TGCTGAACGACATTTTTCTTGCTGACCATCGCTACCGCGAG | |
| | | GTAACTCTTAGTCTTACCGCTAATGAAAATTATCCTTCAGAG | |
| | | CTTGTACGTGTTACGTCCGGAAGTACCGCCGGGGCTTTTTA | |
| | | TCATGTGAGCTTCCCGTTCGATGTACCCGATGGAGAATGGC | |
| | | ACTTCCCCGAACCCGGACATATGCACGCGGTGGCGGATAA | |
| | | AGTTCGTAGTTTGGGGAAGTCATTGCTGCATGCACAGACAT | |
| | | TTGATTGGCGTCCAAACGGTGGCTCTGCGGCGGAACAGGC | |
| | | GTTAATGCTTGCGGCCTGTCAACCCGGTGATGGTTTCGTTC | |
| | | ATTTCGCACATGGAGACGGAGGGCACTTCGCCTTAGAGGC | |
| | | TCTGGCATCAAAAGCAGGTATCGAAATCTTTCATCTGCCAG | |
| | | TTGACCCAGACACGCTGCTTATTGATGTGAATCGTTTAGCT | |
| | | ACGTTAGTGGACGCACATCCACGTATTCGTATTGTCATTTT | |
| | | GGACCAGTCATTTAAACTTCGCTGGCAGCCTCTGCGCGCG | |
| | | ATCCGTGATGCACTTCCTGCACATTGTACGTTGACTTACGA | |
| | | TGCTAGCCACGATGGAGGGCTGGTTATGGGAGGATGGTTT | |
| | | GACAGCCCGCTTCGTTGTGGTGCTGACGTAGTTCATGGTAA | |
| | | TACCCATAAAACTATTGCAGGGCCTCAGAAAGCTTATGTTG | |
| | | CTTTTGGCTCTGCTGAGCACCCCTTATTAGCAGATACCAGT | |
| | | ATTTGGGTGTGCCCGAACATTCAGAGCAATTGTCATGCAGA | |
| | | ACAGCTGCCATCTATTTGGGTTGCATTGAAAGAAATCGAAG | |
| | | CATACGGGCCTGCATATGCGTCCCAGGTAGTGCGTAACGC | |
| | | GACAGCGTTTGCTCGTGCTTTACACGCGCGTGGGCTTGAC | |
| | | GTGTCAGGAGAGTCCTTTGGGTTCACCGAAACCCATCAAGT | |
| | | CCACTTCAGCGTCGGGACCCCGGAGGCAGCGTTATTGACA | |
| | | TGTCGTGACGTGTTGCACCGCGGGGGAATCCGTACCACGA | |
| | | ACATCGAGCTTCCGGGTAAGCCGGGGGTACATGGCATCCG | |
| | | TCTTGGAGTACAGGCAATGACGCGTCGTGGAATGGTCGAG | |
| | | CGCGACTTTGAAACCGTCGCCGACTTTATCGCTGCGCTTTG | |
| | | TACACGCAAACGTACACCCGAGGATGTGGCTCCGGATGTC | |
| | | GAAACGTTCCTGGGTGACTTCCCATTATCCCCACTTGCATTT | |
| | | TCCTTCGACGGGGGTATGACTGACGCATTGCGTGCCGCAC | |
| | | TGCGCCAAGGCGTGATGCGTTAA | |
| CsTTA | WP_018749561.1 | ATGACGCGCACGACCCCCCAGGCACGTCATGTCGTGGAGC | 47 |
| | | GCCTGAATTCAGTTTTAGGACAAGACTACCGCTATCGTGAG | |
| | | GATTGTCTGAGCCTTACCGCGAATGAGAACTATCCTTCCGC | |
| | | ATTAGTGCGCTTAGCGGGGAGTGCCACAGCTGGAGCCTTC | |
| | | TACCACTGTAGCTTTCCGTTTGAGGTGCCACCGGGAGAATG | |
| | | GTATTTTCCTGAGAGCGGTCGTATGGGGGAACTTGCTCAAC | |
| | | AGCTGAATGAATTAGGTCGTTCGTTATTAGGCGCGGGTACA | |
| | | TTCGATTGGCGCCCCAACGGTGGCTCGCCAGCGGAGCAGG | |
| | | CATTGATGTTAGCGGCCTGCAAGCACGGTGAAGGGATGGT | |
| | | CCATTTTGCTCATCGTGACGGTGGCCACTTTGCGCTGGAGA | |
| | | ATCTGGCGCAAAAAGCTGGTATCGACATCTTTCATTTGCCT | |
| | | GTAGATCCCCAGACGTTGTTGATCGATGTTGCACGCCTTGA | |
| | | CGAGCTTGTCCGCCGCAATCCTCAAATCCGTATTGTGATCT | |
| | | TGGACCAGTCTTTTAAGTTACGCTGGCAACCCCTTGCAGCG | |
| | | ATCCGCAAGGTTCTTCCCCCATCGTGTACACTTACCTATGA | |
| | | CACCTCTCATGATGGTGGACTTATTATGGGAGGAGTTTTTG | |
| | | ATTCTCCCTTGCATTGTGGTGCAGACGTAATTCATGGCAAC | |
| | | ACGCATAAAACAGTGCCCGGACCGCAGAAGGGGTATATCG | |
| | | CCTTCAAATCCGCTGAGCATCCTTTGTTGGTTGACACGAGT | |
| | | CTGTGGCTTTGCCCACATTTGCAGTCTAACTGTCATGCCGA | |
| | | GCTTTTGCCTCCAATGTGGGTGGCTTTTAAAGAAATGGAGG | |
| | | CTTTCGGACATGATTACGCCCCTCAAGTGGCCCGCAACGC | |
| | | GAAGGCTCTGGCGGGTCATTTACATCGTTTAGGATTCGAGG | |
| | | TTTCAGGCGAGGCTTTCGGTTTCACTGAAACCCACCAAGTG | |
| | | CATTTTGCCGTAGGAGACTTGCAGCAAGCGCTTGATTTGTG | |
| | | CATGAACACCTTGCATCGTGGGGGCATCCGCTCTACGAATA | |
| | | TTGAAATCCCGGGTAAACCCGGCATTCAGGGTATTCGCCTG | |
| | | GGCGTTCAGGCTATGACCCGTCGCGGTCTGCGCGAAGATG | |
| | | ATTTTGAGCAGGTGGCGCGTTTTATCGCGGACTTGCACTTC | |
| | | CGCAAAGCAGACCCAGCCGGAGTCGCAGCACAAGTAGCGG | |
| | | AATTTCTTCGTGCTTTTCCTTTGGCACCATTACATTACTCATT | |
| | | TGATCAGGAACTGGATCATGAGTTATTGCAGTCCCTTATTG | |
| | | GGGAGGCGTTACGCTAA | |
| BuTTA | WP_080410754.1 | ATGATGACGGACTTCGCACAGGCGGTAGTAAACCCGTTCG | 48 |
| | | TAGATGAGCAGCGTAAGTCCCGTTTAGTAGAAAAAATCTCA | |
| | | AACATCTTCGATAGTCTTCATAGCGATTTTGCCTTGGATAAT | |
| | | TTATACCGCGCAAGCCACTTAAGTCTGACCGCCTCTGAGAA | |
| | | TTATCCATCCCGCTTTGTGCGCACGCTGGGAGCCGGTATGC | |
| | | AAGGCGGTTTCTATGAATTCGCGCCACCTTACGCCGCTAAC | |
| | | CCAGGAGAGTGGTACTTCCCTGACAGTGGCGCGCAGTCGA | |
| | | GTCTGGTCGAGAAACTTGCTAGTTTGGGAAAACAGTTGTTC | |
| | | GAGGCTAACTCGTTTGACTGGCGTCCCAACGGGGGATCAG | |
| | | CAGCGGAACAGGCTGTGCTTTTAGGCACATGTGCCCGCGG | |
| | | GGATGGCTTCGTCCACTTTGCTCACAAGGATGGCGGCCAC | |
| | | TTTGCTCTGGAAGAGTTGGCCCAGAAGGTGGGAGTTAGCA | |
| | | TCTTCCATCTGCCAATCGAGGAGAAGAGTCTTTTGATTGAT | |
| | | GTTGACCGCCTGGCGACATTAATCAAAGATAACCCCCACAT | |
| | | TAAGCTTGTAATTCTGGACCAATCGTTTAAGCTTCGCTGGC | |
| | | AACCTTTACTGCAAATCCGCCAAGCCTTACCGGAATCAGTC | |
| | | GTATTATCGTACGACGCGAGTCACGACGGGGGATTAATCAT | |
| | | CGGCGAATGCCTGCCCCAGCCATTACTTTTCGGAGCGGAT | |
| | | ATTGTTCACGGGAATACACACAAGACAATTCCGGGCCCGCA | |
| | | AAAGGGTTACATTGCGTTCAAGAATGTAGACCATCCTGCGA | |
| | | TGAAGCATGTTAGCGATTGGGTTTGTCCTCATTTGCAATCT | |
| | | AACTCGCATGCCGAGTTGATCGCACCCATGTATATTGCCTT | |
| | | GGTTGAAATGTCTTTGTACGGACGCAGTTACGCGGAGCAG | |
| | | GTTATTAAAAATGCTAAGGCGTTGGCACACGCCCTGCACGC | |
| | | CGAGGGAGTACGCGTCTCGGGCGAATCGTTCGGTTTTACA | |
| | | GAAACACACCAAGTTCATGTTGTTGTTGGGTCCGAGCGTAA | |
| | | AGCGTTGGAGTTAGTTACTGGTACCTTGGCATTGGCAGGAA | |
| | | TTCGCTGCAACAACATCGAGATTCCAGGCGCGAACGGCTTA | |
| | | TTTGGTTTGCGCTTAGGAGTGCAGGCATTGACGCGTCGCG | |
| | | GAATTAAAGAGCACGGGATGGCTGAAGTTGCCCGTTTTTTA | |
| | | GTGCGCTTGATTCTGAAAAACGAATCCCCCACGGCCATCCG | |
| | | CAACGAAATTGCGTCATTTCTTGAATCATATCCTATTAATAC | |
| | | GCTTCATTATTCATTAGATGCTCACTATTATACCCCTTCGGG | |
| | | TATTAAATTGATGGAGGAAGTAATCGCTTAA | |
| StTTA* | WP_101279775.1 | ATGGGAGTTTGGGCAGGCGACCGTGTTGCCCAAGTTTTGG | 49 |
| | | AACGCTTAGCGTCGGATTTTGTTTTAGACAACACTTATCGC | |
| | | GAACAACACCTGAGCTTGACGGCTTCTGAGAACTATCCTTC | |
| | | AAAACTGGTACGCATGTTGGGAGCGGGATTACAGGGGGGT | |
| | | TTCTATGAGTTTGCTCCGCCCTATCCGGCAGAAGCAGGAGA | |
| | | ATGGGCATTCCCGGACTCCGGAGCGAACGCGTCCCTTGTA | |
| | | GGGAAGCTGACTGGCATTGGTCGCCAACTGTTCGAAGCCG | |
| | | CAACATTCGACTGGCGTCCGAACGGCGGATCCGTGGCCGA | |
| | | GCAAGCAGTATTGCTGGGGACGTGTGGACGCGGGGATGG | |
| | | TTTTGTGCACTTCGCGCATAAGGATGGGGGCCACTTTGCGT | |
| | | TGGAGAGTCTGGCGGGTGCTGCCGGAGTCAACACGTATCA | |
| | | TCTGCCCATGGTAGACCGCACGCTTCTGATCGATGTCGATC | |
| | | GTTTGGCTACTTTATGCGCTGAACACCCGGAAATTAAGTTA | |
| | | GTAATCTTAGATCAGTCCTTCAAATTACGCTGGCAACCGCT | |
| | | TGCTCAAATCCGCGCCGCGCTGCCCGAGGGCGTATTTTTA | |
| | | GCTTATGACGCGTCTCATGACGGTGCTTTGATTGCTGGGG | |
| | | GTGTTCTGCCACAGCCTACCCTGTTAGGGGCCGATGCAGTT | |
| | | CATGGCAACACGCACAAAACGATCGCGGGGCCTCAAAAGG | |
| | | CGTATATTGCGTTCCGCGACGCTGAGCACCCCAAGTTACGT | |
| | | GCCGTCAGTGATTGGGTGTGTCCACAGATGCAGAGTAATTC | |
| | | ACATGCGGAACTGATCGCACCCATGTATGTAGCACTGTCGG | |
| | | AGGTCGCCTTATATGGTCATGCGTATGCCCGCCAAATCTTA | |
| | | GCAAACGCCCAAGCGTTAGCGCACGGATTACACGAAGAGG | |
| | | GGGTCCGCGTATCTGGAGAGTCCTTCGGCTTTACAGAAACT | |
| | | CATCAAGTACACGTCGTGACGGGTTCAGCTGCGGATGCTCT | |
| | | GCGCCTGTCCTTGGGTGAGCTGGCCCAGGCAGGAATCCGT | |
| | | ACGACAAACATTGAGGTACCAGGGGCAAATGGACTGCATG | |
| | | GTTTGCGCTTAGGAGTTCAAGCTATGACTCGCCGTGGTTTA | |
| | | CGCGAGCCACAGATGCGTGAAGTGGCACGCTTGGTTGCCA | |
| | | AAGTTGTTTTGCGCCGTGCCGAACCAGCGGCTGTACGCGC | |
| | | GGAGGTTGCGGATTTGTTACAGCATCACCCGTTAGATCAGT | |
| | | TGGCGTATTCCTTCGATTCCTACGTTGACTCGCCAGCTGCG | |
| | | GCGCGTTTGTTGGGTGAGGTCTTTCGTTAA | |
| TmTTA | WP_188596100 | CCATCACCATCATCACCACATGCGCGAGGAAGAAGCGATT | 50 |
| | | GCGGCGCTGTCAAAATTACGCGCAATCATGGACCGCCATA | |
| | | ACAACTGGCGCCGCCGTGAGACAATTAACTTAATTCCAAGC | |
| | | GAAAACGTGATGTCGCCGTTAGCCGAGTATTTCTACTTAAA | |
| | | TGATATGATGGGACGTTATGCTGAAGGAACGATTGGTAAAC | |
| | | GCTACTACCAAGGTGTATCGCTGGTGGACGAGGCGGAACA | |
| | | AATGTTAGTCGATTTAATGAGCTCTTTGTTTTCCTCGCGCTT | |
| | | TACAGACGTCCGCCCCATCAGCGGTACAGTTGCCAATATGG | |
| | | CCGTGTATCACTCAGTCGCGGGGCTTGGGGAGAAGATCGC | |
| | | CTCTTTACCAACAGCCGCCGGGGGCCATATTTCGCATAACG | |
| | | AGACTGGTGCCCCCAAAGCATTCGGATTACGTGTTTCATAT | |
| | | TTGCCGTGGTCTCAGGAAAACTTTAACGTGGATGTGGACGC | |
| | | TGCGCGTCGCTTAATTGCCGAAGAACGCCCAAAATTGGTGT | |
| | | TGCTTGGGGCGTCACTTTATTTATTTCCTCATCCCATTAAAG | |
| | | AATTAGCGGACGCTGCTCACGAGGTAGGTGCGGTTCTGAT | |
| | | GCATGACTCAGCTCACGTACTTGGTTTAATTGCTGGTCATC | |
| | | AGTTCCCTAATCCTCTTGAACTTGGGGCGGACATTATGACT | |
| | | AGCAGCACGCACAAAACTTTTCCGGGACCCCAAGGCGGTG | |
| | | TGATTTTTACCACACGTGAAGATTTGTTCAAGGAGATCCAA | |
| | | CGCTCAGTTTTCCCAGTAATGACATCGAATTATCACTTGCAT | |
| | | CGCTATGCCTCGACGATTGTGACAGCTATTGAGATGAGTAC | |
| | | GTATGGAGACGAATATGCAGCTACAGTGCGCTCCAACGCG | |
| | | AAAGCACTGGCGGAACAACTTCATGCCAACGGTTTACCTGT | |
| | | AGTTGCCGAAGAACACGGCTTCACGGCTACCCACCAGGTG | |
| | | GCAATGGATGTTTCAAAATTTGGAGGCGGGGGGCCAATCG | |
| | | CTAAAGCGTTGGAGGACGCGAATATTATTGTAAACAAGAAC | |
| | | ATGCTGCCCTGGGATAAGTCTCCGGTCAAACCATCCGGTAT | |
| | | TCGCATGGGAGTTCAAGAAATGACTCGCATGGGAATGGGT | |
| | | AAAGGCGAGATGGCGGCCGTGGCGGAGCTGATCGCAAAG | |
| | | GTGGTCATCAAAGGGGTCGAACCGTCTAAAGTAAAGCCAG | |
| | | AGGTCGTCGAGTTGCGCCGCGGTTTCACAAAGGTACGCTA | |
| | | TGGTTTTGATTTATCTACTTTGGGCTTGAATTGCCCTTGTCT | |
| | | TCCGTTACTGTAACTTGATGGGGGATCCCATG | |
| RaTTA | GIH11859 | CCATCACCATCATCACCACATGTTGGAAATTGTGGGGGACC | 51 |
| | | ATGAACGCAAAATGGCGAGTGCAGTGAATCTTATCCCCAGC | |
| | | GAGAATTTATTAACACCCGCCGCACGTTTAGCCTACCTTTC | |
| | | AGATGCGTATTCGCGTTATTTTTTCGATGAGCGTGAGGTGT | |
| | | TCGGAAAGTGGTCTTTCCAAGGGGGGAGCATTGTGGGCGA | |
| | | AGTACAACGTGAGGTTTTAGTGCCTCTGGTACAAAAGGTAA | |
| | | CTGGGGCACGCCATGTGGACGTCCGTGGGATTAGTGGCCT | |
| | | GAATGCCATGACCGTGGCTCTGGCAGCGTTTGGCGCCCGT | |
| | | GACCGCGTTACAATTACAGTACCGCCCCGCCACGGAGGCC | |
| | | ATCCAGCTACCGCAGTTGTGGCCGGACACTTTGGGCATCG | |
| | | TGCAGAGGCTTTACCTTTCCGTGATGAAGCCTGGTGGGAG | |
| | | GTTGACTTGCCTGCCTTAGCGGAGTTAGTAGCTCGTACTGA | |
| | | TCCGGCGTTAGTTTATGTAGATCAGGCCACCGCTCTGGTCC | |
| | | CACTGGATTTAGCCGGAGTAATCCGCACCGTCAAGGAAGTT | |
| | | TCCCCTGGGACACACGTACACGCCGACACATCGCACATCAA | |
| | | CGCGTTCGTTTGGTCGGGATTGTTCGGCCAACCACTTGACT | |
| | | TGGGGGCGGACAGTTACGGAGGCTCCACGCATAAGACCTT | |
| | | TGCGGGCCCTCATAAGGCTTTATTGCTTACTAACGATGACG | |
| | | CAGTGAGCGATAAACTGACCTCCGTCGCAGTGAATCTTGTT | |
| | | TCGCATCATCATGTCAGCGACGTTGTAGCTTTAGCTATCGC | |
| | | CATGGTAGAGTTCGCGGAATGTGGCGGGGTAGATTACGCG | |
| | | CAGGCAGTTTTAGCAAATGCAGCGGCGTTCGCCCGCGCCC | |
| | | TGGCCGATGCCGGGCCTGGCGTACAAGACGCGGGTGGTG | |
| | | TCTTAACCCGTACGCATCAAGTATGGTACGAACCTGCTGGC | |
| | | GATCCGCACCGCATTAGCGAGCGCTTGTTCGATGCGGGGA | |
| | | TCGTTGTGAACCCTTACAACCCTCTGCCGAGTACCGGTCGT | |
| | | TTAGGAATCCGTATGGGGTTAAATGAGGCGACCAAGTTAG | |
| | | GATTCGGAGAACCGGAAATGGCCGAGTTAGCAGGGTTGCT | |
| | | TCACGGTGTAGCGGTTGACCGTATCGCCGTGGCTGAGGCG | |
| | | GGAGAGCGTGTGGCTGCCATGCGTCAAGCCGCTCGTCCCG | |
| | | CGTATTGTTTTTCTGAAGATGTGGTCGCCTCTAAGCTTCGC | |
| | | GAGCTTACCGGAGCCTCAGGTGCAGGTGTGGATGAGTTGG | |
| | | CTGCGTGGCTTTATCGTTAACTTGATGGGGGATCCCATG | |
| SNTTA | ADZ45329 | CCATCACCATCATCACCACATGACATCAAGCGACGATTGTG | 52 |
| | | CTGCGAGTCGTACGGCTCCCGTCGCTGGCCGCGCAGAACT | |
| | | TTTGGCGCTGTTGGGAGAAATCGAGAAGGAGCAGCGCATC | |
| | | AACGAGGCCGCCGTGAACTTAGTGCCTTCAGAGAATCGCA | |
| | | TTAGTCCCTGGGCTGGGGCGCCGTTACGTACCGATTTTTAC | |
| | | AACCGCTATTTCTTCAACGATTCTCTGGACCCCCAGGGATG | |
| | | GCAATTTCGTGGAGGGGAAGGGATTGGACGCCTGGAAAAG | |
| | | GAGTTGGCTCTGCCCGCTTTACGCCGTTTAGGGCGTGCCG | |
| | | ATCACGTTAACATCCGTCCTGTGTCAGGTATGAGTGCCATG | |
| | | CTTGTGGTCCTTTTAGGTTTGGGAGGCGAACCTGGGGATG | |
| | | GTGTAGTGTGTGTAGACGCAGAAACGGGAGGTCATTATGC | |
| | | TACTGGCCGCCAAATCGCAATGTTAGGCCGCCGCCCTTTGC | |
| | | CCGTCCGCGTGGTAGCGGGACGCGTTGATTTGGATGCTCT | |
| | | TCGCACGGCATTAACTAGCTGCCACGTTCCCTTGGTATATC | |
| | | TTGACCTTCAGAATTCACTTTGGGAGCTTGATGTTGCGGGA | |
| | | GTAGCCGAGGTCATCGCACGTACAAGCCCACGTACTGTTCT | |
| | | GCACGTGGACTGCAGCCACACATTAGGATTAATCCTTGGG | |
| | | GGCTCACATAAAAATCCATTAGACTTGGGTGCGGATACGAC | |
| | | TGGGGGGTCGACCCATAAAACTTTCCCAGGTCCGCAGAAA | |
| | | GGGGTTTTGTTCACACGTGACGAGAACTTGAGTCGTAAGAT | |
| | | CCGTGATGCTCAATTTTTCACGATCAGTTCACATCACTTCGC | |
| | | GGAAACACTGGCGTTGGCCTTAGCGGCTGCAGAATTTGAG | |
| | | CATTTTGGCGCAGCCTATAGCCGCCAAGTCCTTATCAATGC | |
| | | TCGCGCTTTTGCACACCGCTTACGCGAGCGCGGATTTGGA | |
| | | GTCGTTGAAGGCGGCCCGCAGCTGACGGATACTCACCAAG | |
| | | TCTGGGTCCGCTTACCTCTTGAAGAATCGGCAGATGCCTTT | |
| | | AGCGCTCAATTGGCGTCCTTAGGTATCCGCGTCAATGTCCA | |
| | | GACTGAGTTGCCAGACATCCCTGAACCAGCCCTGCGCTTAG | |
| | | GCGTGAGCGAGATTACTCTTAATGGTGGACGTGAGCCAGC | |
| | | AATGGAAACGTTGGCAGAGATCTTCGCTTTGGTACGCGCA | |
| | | GGGGAGGCGACTAAGGCTGTCGATTTATTCCAAGTTCTTCC | |
| | | CCATGAAATGGGGGAACCGTATTTTTTTACGGGATTACCTC | |
| | | AAGAAGCGGGACTTTTTCATGGGTAACTTGATGGGGGATC | |
| | | CCATG | |
| NoTTA | WP_052373448 | CCATCACCATCATCACCACATGAATACGTTCGATATCTTAGA | 53 |
| | | ACAACTTGCACGTTATGAGGTAGGCACATCGCGCCGTTTGC | |
| | | ATTTAATTGCGTCTGAGAATCCCCTGGACTCAGACACACGT | |
| | | GTGCCGTATATGCTTGCAGGAACTTTAGCTCGTTACGCATT | |
| | | TGGGGAGCCGGGTCAGCCCAACTGGGCTTGGCCAGGCCG | |
| | | TGAGACTCTGATTGACCTGGAAGCTGACACTGCGGCAGCC | |
| | | CTTGGGGCTTTGCTGGGCGCCGATCATGTTAATCTTCGTCC | |
| | | GACTAGTGGTCTTTCAGCTATGACCGTGGCCTTGTCCGCCT | |
| | | TGGCCGAACATGCTGGGGACCGTGCAACTGTTTTATCGCTT | |
| | | GCAGAATCAGATGGTGGCCATGGATCGACGGGGTTCATGG | |
| | | CCCGTCGTTTTGGGCTGGACTGGCAACGCATGCCCGCTGA | |
| | | CCCGCGTACAGGCGTTGTGGATCTGGACGCACTGGCGCGT | |
| | | CAGGCTCGCAGTGCCCGCGGTCCTCTGGTCTTATATCTGGA | |
| | | TGCGTTCATGGCGCGCTTTCCTTTTGACTTAACGGGTATCC | |
| | | GCGGTGCGGTGGGTGACTCAGCTTTGATCCATTACGACGG | |
| | | TTCACATCCTTTGGGATTAATCGCGGGAGGCCGTTTCCAAA | |
| | | ATCCGTTAGCTGAAGGCGCCGATTCGCTTGGAGGGTCTGT | |
| | | ACACAAAACCTGGCCTGGACCGGTAGGGAAAGGGATCATC | |
| | | GCTACCAATGATAGTGCACTTGCATCTCGCTTCGATACTCA | |
| | | CGCCGCGGGTTGGATCTCCCACCATCACCCTGCGGATCTG | |
| | | GCTGCACTGGCGCTTAGTACCGCCTGGATGGAGCAACATG | |
| | | CTGGCGACTACGCGACAGCAGTGATCGCAAATGCCGTGCA | |
| | | ATTAGCTGATGAACTTGCAGACGGCGGCTTGAGCATCTGTG | |
| | | CCGATGACCGTGGTGCTACGGCGAGTCATCAAGTGTGGGT | |
| | | TGATATTGCTCCTATCTGTCCAGCTCCTGTCGCGGCTCAGC | |
| | | GTTTGTATGATGCTGGTATTGTGGTAAACGCGATTGCAATC | |
| | | CCAGGGCTTGCCGAACCCGGCTTGCGCCTGGGCGTTCAGG | |
| | | AGTTGACTCGCTGGGGATTAGACCGTGATGGAATGACAGT | |
| | | CCTGACCTGGGTACTGACCCAACTGCTGGTCCATAACGCG | |
| | | GCCACAGCAGTGGTGGCCCCGCAAATGGAAGCGTTGCGTA | |
| | | CCGGCCTGACGCTGCCTGAAGATCGTCATGGGCTGGAGGG | |
| | | TTTTCTTCGTGCGTGTGATCCACAGGAGGTATCAGTCGCAT | |
| | | AACTTGATGGGGGATCCCATG | |
| KaTTA | WP_033354341 | CCATCACCATCATCACCACATGGATGTGTTGGCTGCCCTGG | 42 |
| | | AACGTAAGCACAGTTTAAACTTGTTTCCGATTGAAAATCGCT | |
| | | TGTCACCCCGTGCTGCCGCCGCTCTGGCATCCGATGCCGT | |
| | | AAACCGTTATCCGTACAGTGAGACGGATGTGGCGGTGTAC | |
| | | GGAGACGTTAGTGATCTGAATGCTGTATATGACCATTGCGT | |
| | | CAGTCTTACCAAGGAATTTTATGGCGCCCGTCATGCATATG | |
| | | TTCAGTTTCTTTCCGGACTTCACACCATGCATACAGTGTTAA | |
| | | CAGCAGTCACACCGCCAGGGGGCCGTGTAATGGTCATTGC | |
| | | GCCTGAAGACGGAGGACATTATGCAACGGTTACTATTTGCC | |
| | | AAGGTTTTGGCTACCGCGTAGAGTACGTACCATTCGATCGC | |
| | | CAGACTTTGGAAATTGACTACACTGCTCTTGCCGAACGCAC | |
| | | AGCCGAACATCCGGCTGATGTGATCTACTTGGACGCATCGA | |
| | | CGGTATTGCGCATGCCTGACGCGCGCGCTCTGCGTGCAGC | |
| | | AGCCCCAGGCGCTGTTCTGTGTCTGGATGCAAGTCATCTTC | |
| | | TGGGACTTCTTCCCGCAGCCCCTGGGACCTTGGTCCTTGAT | |
| | | GCTGGCTTTGATTCAATTTCTGGAAGCACTCACAAAACTTTA | |
| | | CCGGGACCCCAAAAGGGATTGTTGGTGACAAACTCCGATG | |
| | | CCATTGCCGAACAGGTCGGAGCGCGCATCCCTTTTACCGC | |
| | | GAGTTCATCGCATTCTGCGAGCGTGGGTTCGCTGGCGATT | |
| | | ACATTAGAAGAGCTTTTGCCCCATCGCGGGGATTACGCACG | |
| | | TCAGGTGATCGCAAACGCCCGTGAGCTGGCTCGTCAACTT | |
| | | GCGGCCCGCGGCTTTGACGTGGCAGGGGAAGCCTTCGGAT | |
| | | TTACTGATACTCATCAGGTGTGGGTCCACCATCCAGAGGGA | |
| | | AATACACCGCATGAGTGGGGACGTCTGCTGACAGCTACTG | |
| | | ATATTCGCACCACTACAGTAGTGCTTCCATCAACTGCACGT | |
| | | AGTGGATTACGTTTAGGAACGCAGGAGTTGACACGTTGGG | |
| | | GGATGAAGGAAGACGATATGACTACCGTTGCAGAGCTTCTT | |
| | | GCCCGTCTGCTTTTACGCGGAGAACAGAGTCGCTCAGTTG | |
| | | CCGCGGATGTACGCGACTTGGCTCGTTCGTTCCCAGGTGT | |
| | | GGCTTTCGCGGACCGTCCAGCACCCTTGGCAGTAGCCTAA | |
| | | CTTGATGGGGGATCCCATG | |
| PbTTA | MBN2478762.1 | CCATCACCATCATCACCACATGGAAACCTCCCTGAAGGATT | 43 |
| | | TTGAAACTATCCTTCACTTAATTAATAAGGAGGAGATTGACT | |
| | | CAAATGACACCATTCATATGACCGCCAACGAAAATATTATG | |
| | | TCTAAATTGTCCAAACACTACTTAAAAAGCACTTTGTCTTAC | |
| | | CGCTACCATGTCGGAATGTTCGATGATCAAAAGAACCTGAC | |
| | | AGTCTCGCGTTCGTGTCTTATCAAAAACTCTTTGATGCTGC | |
| | | GTTGCCTTTCACCCATCTTCCTGTTAGAACAACAAGCCCGT | |
| | | GAATACGTAAAAAAAATGTTCTTCGCTGAGTATGCGGACTT | |
| | | TCGTCCTTTGTCCGGTATGCACACCGTTTTTTGTATCTTATC | |
| | | TACCTTAACAAAACCGAACGATCGTGTCTATGTCTTCACGA | |
| | | CCGAATCGGTAGGACACGCAGCCACAGTTTCTTTATTGAAG | |
| | | TCGTTGGGTCGCAAAGTGTCCTTCATCCCATTTTGTGAGAA | |
| | | GAAACTTGATATTGACTTAGAGAAGCTGAGTAAACAAATCT | |
| | | TGATTGAGAAACCCAACGCAATTCTTTTTGATTTTGGTACTC | |
| | | CATTCTACCCATTGCCGATCCGCGAAATTCGCGAGATTGTA | |
| | | GGAAACGACGTGAAGATGATTTATGACGCCTCGCATGTGTT | |
| | | GGGTTTGATTGCGGGTGGACAGTTCCAAAATCCACTTCTTG | |
| | | AAGGCTGTGACGTGCTGATCGGAAATACTCACAAGACATTT | |
| | | CCGGGGCCGCAGAAAGGCATGATCTTGTATAAAAACAAGT | |
| | | CTTTGGGAAAGGAGATCGCAACAGAAATTTTCAAATCAGCC | |
| | | ATTTCTGCGCAGCATACTCATCATGCTATCGCCCTGTACGTT | |
| | | ACTATCATTGAAATGTATATCCACGGGAAGGAATACGCCAA | |
| | | CCAAATCATCAAAAATAATCATGCGTTATCCCAGGCATTAAT | |
| | | CAATGAAGGTTTTAAAATTTTTAAGCGTAAAAACCAGTTTAG | |
| | | CCTTAGTCACATGATTGCGATTACGGGGGATTTTCCGATTG | |
| | | ATCATCATGTTGCATGTGCCGATTTGCATAATTCTAACATCT | |
| | | CCACAAATTCGCGTATTCTGTATGACTTTCCAGCCGTGCGC | |
| | | ATTGGCGTTCAGGAGGTTACACGTAAAGGAATGAAAGAAA | |
| | | AGGATATGGTGCAATTAGCCAAATTTTTTAAGGAAATCATC | |
| | | CTGGATCGCAAGAACATCAGCTCTAAAATCAAGGAGTTCAA | |
| | | TAACAAATTCAATAGTATTGAATATAGTCTTGACGAGATCTA | |
| | | CGAGAAGTTATTCTAACTTGATGGGGGATCCCATG | |
| DbTTA | MBI5609283 | CCATCACCATCATCACCACTTGACGAATAATCGCGAGCTTA | 54 |
| | | TGGACCGTATCGGTTATAATCTTTCACAAGGTTTAGTTTCAA | |
| | | GCCAGCATACCGCAAGTCTGGTCGCTTTATTTATTGCATTA | |
| | | CATGAAGCACGCCTGACCGGCAAAGCGTTCGCAAAGCAAG | |
| | | TGGTAGAAAACGCCCGTACGTTGGCGAGTCGTTTGGCGGC | |
| | | ACTTGGCGTTCCGGTGTTAGCGCGTTCAGATGGCCAGTTTA | |
| | | CCGACAATCATCATTTCTTCATCAATTTGACCGGCGTGGCG | |
| | | AGTGCTCCTCACCAAATGGAGCGCTTACTTCGTGCCCATTT | |
| | | GGTTGTTCAGCGCGGCATGCCGTTTCGCAACGTTGACGCC | |
| | | TTGCGTGTTGGCGTGCAAGAAGTCACACGCCGCGGTTATG | |
| | | GACCCGGCGAGATGGCGCAGCTGGCAGAGTGGATTGCGT | |
| | | CAATCGTCATCGGCGGTGCGGACCCCGAGGTAGTAGCACC | |
| | | TGCCGTGCAAGCCATGGCTAAGCGCTTTGACACTATCTATT | |
| | | ATACGGGCGAAACGGTGGACGGTAAACTTGATCTTCCAGA | |
| | | AATCGCAGCGCCGAGCGCTAAGGGCCGTTGGGTTGACTAT | |
| | | CGCCATTTGGGAAATGATTTTGCAATGGACGATACTGAGTT | |
| | | CTCCGAAATTCGCGCCTTGGGTGCTGCCGCGGGAGCCTTC | |
| | | CCAAACCAGACCGACAGTACAGGTAACGTCTCGTTACGTTC | |
| | | AGGAGCCCGTGTATTCGTGTCGTCTAGCGGGTCATATATTA | |
| | | AGCACCTGGCCGACGGACAGGTCGTCGAGTTGGACGCGGT | |
| | | AGATCCCTCAGGGGAATTGATTGACTATCATGGTGCGGCGT | |
| | | TGCCCAGCAGTGAGAGTCTGATGCACTTCTTAGTTTACCAG | |
| | | AATGTGCCAGCGGGCGCAGTTGTGCACACTCACTATTTATT | |
| | | AACCAACCAAGAGGCTGCCGACTTCGATGTGGCGGTGATC | |
| | | GCTCCTCAGGAATATGCCAGTATTGCACTTGCCCGCGCAGT | |
| | | AGCAGAAGCCAGTAAACGCTCCCGTATCGTGTATATTCAAA | |
| | | AACACGGATTAGTGTTTTGGGGTACAGACACTGCAGATTGT | |
| | | CTGTCTCAGGTTCACAACTTTATTCACAACCGTCCAAATCGT | |
| | | CGCGCAGCTGAGGCGGTCTATGCCTCTTAACTTGATGGGG | |
| | | GATCCCATG | |
| SUMO | | ATGTCCCTGCAGGACTCGGAGGTTAACCAGGAAGCAAAGC | 55 |
| tag | | CGGAAGTCAAACCGGAAGTGAAACCCGAAACTCACATCAAT | |
| | | CTGAAGGTAAGTGATGGTTCTTCAGAGATATTCTTTAAAATT | |
| | | AAAAAAACCACGCCTCTGCGGCGTCTTATGGAAGCGTTCGC | |
| | | CAAACGACAAGGGAAAGAGATGGATAGCTTACGTTTTCTCT | |
| | | ATGATGGCATTCGCATCCAGGCGGATCAAGCTCCAGAGGA | |
| | | CTTGGATATGGAAGATAACGACATTATCGAAGCCCATCGCG | |
| | | AACAGATTGGTGGC | |

⁺Start codons for each gene are underlined.
*For StTTA, the first 36 amino acids at the N-terminus were removed to improve the similarity between StTTA and ObiH.

TABLE 4
Absorbance of Investigated Aldehydes

| | Abs at 1 mM | Final concentration |
|---|---|---|
| Aldehyde | (340 nm) | in ADH assay (mM) |
| 1 | 0.2452 | 1 |
| 2 | 0.3799 | 1 |
| 3 | 0.4418 | 1 |
| 4 | 0.3092 | 1 |
| 5 | 4 | 0.25 |
| 6 | 0.2291 | 1 |
| 7 | 0.2612 | 1 |
| 8 | 0.2291 | 1 |
| 9 | 0.2412 | 1 |
| 10 | 0.6106 | 1 |
| 11 | 0.2952 | 1 |
| 12 | 0.7088 | 1 |
| 13 | 0.2328 | 1 |
| 14 | 0.244 | 1 |
| 15 | 0.3858 | 1 |
| 16 | 0.4201 | 1 |

TABLE 5
Predicted Attributes of Selected Threonine Transaldolases

| | | | | | | antiSMAS |
|---|---|---|---|---|---|---|
| | | | | | | H Most |
| | | | | Host | | similar |
| | | | | Genome | | known |
| | | | | Assembly | antiSMASH | cluster |
| Threonine | Accession | Host | | for | BGC | (% |
| transaldolase | Number | Organism | Class | antiSMASH | Type | similarity) |
| ObiH | ARJ35753.1 | Psuedomonas | Bacteria | | Obafluorin | 100% |
| | | fluorescenes | | | | |
| PiTTA | WP_095149064.1 | Pseudomonas_{—} | Bacteria | NZ_FYDV01000019.1 | Obafluorin | 85% |
| | | sp._Irchel_{—} | | | | |
| | | s3a18 | | | | |
| BsTTA | WP_060149112.1 | Burkholderia | Bacteria | NZ_QTPN01000035.1 | Obafluorin | 71% |
| | | stagnalis | | | | |
| CsTTA | WP_018749561.1 | Chitiniphilus | Bacteria | NZ_KB895358.1 | Obafluorin | 85% |
| | | shinanonensis | | | | |
| | | DSM 23277 | | | | |
| BuTTA | WP_080410754.1 | Burkholderia | Bacteria | NZ_MECN01000006.1 | N/A | |
| | | ubonensis | | | | |
| StTTA | WP_101279775.1 | Streptomyces | Bacteria | NZ_CP031742.1 | N/A | |
| | | (multi- | | | | |
| | | species) | | | | |
| TmTTA | WP_188596100 | Thermocladium | Archaea | NZ_BMNL01000002.1 | N/A | |
| | | modestius | | | | |
| RaTTA | GIH11859 | Rugosimonospora | Bacteria | BONZ01000001.1 | Spicamycin | 27% |
| | | africana | | | | |
| SNTTA | ADZ45329 | Streptomyces sp. | Bacteria | HQ257512.1 | Muraymycin | 100% |
| | | NRRL 30471 | | | | |
| NoTTA | WP_052373448 | Nocardia | Bacteria | JADLPU010000004.1 | N/A | |
| | | otitidiscaviarum | | | | |
| KaTTA | WP_033354341 | Kitasatospora | Bacteria | NZ_JNWR01000048.1 | Valclavam | 64% |
| | | aureofaciens | | | | |
| PbTTA | MBN2478762.1 | Parachlamydiales | Bacteria | JAFGQY010000010.1 | N/A | |
| | | bacterium | | | | |
| DbTTA | MBI5609283 | Deltaproteobacteria | Bacteria | JACRCU010000288.1 | N/A | |
| | | bacterium | | | | |

TABLE 6
KaTTA Similarity

| | | % | | |
|---|---|---|---|---|
| | Protein | Identity | | SEQ |
| | Accession | to | | ID |
| Species | No. | KaTTA | Sequence | NO |
| Kitasatospora | WP_033354341.1 | 100% | MDVLAALERKHSLNLFPIENRLSPRAAAALASDAVN | 1 |
| aureofaciens | | | RYPYSETDVAVYGDVSDLNAVYDHCVSLTKEFYGA | |
| | | | RHAYVQFLSGLHTMHTVLTAVTPPGGRVMVIAPED | |
| | | | GGHYATVTICQGFGYRVEYVPFDRQTLEIDYTALAE | |
| | | | RTAEHPADVIYLDASTVLRMPDARALRAAAPGAVL | |
| | | | CLDASHLLGLLPAAPGTLVLDAGFDSISGSTHKTLP | |
| | | | GPQKGLLVTNSDAIAEQVGARIPFTASSSHSASVG | |
| | | | SLAITLEELLPHRGDYARQVIANARELARQLAARGF | |
| | | | DVAGEAFGFTDTHQVWVHHPEGNTPHEWGRLLTA | |
| | | | TDIRTTTVVLPSTARSGLRLGTQELTRWGMKEDDM | |
| | | | TTVAELLARLLLRGEQSRSVAADVRDLARSFPGVAF | |
| | | | ADRPAPLAVA | |
| Streptomyces | EFG04558.1 | 77.95 | MKSVRRRRSPSDSVPFRPPIRGESMDVLAALERKP | 2 |
| clavuligerus | | | SLNLFPIENRLSPRASAALATDAVNRYPYSETPVAV | |
| | | | YGDVTGLAEVYAYCEDLAKRFFGARHAGVQFLSGL | |
| | | | HTMHTVLTALTPPGGRVLVLAPEDGGHYATVTICR | |
| | | | GFGYEVEFLPFDRRTLEIDYAVLAARLSRRPADVIYL | |
| | | | DASSILRFIDARALRLAAPDALICLDASHILGLLPVA | |
| | | | PQTLVLDGGFDSISGSTHKTFPGPQKGLLVTDSDV | |
| | | | VAEKVAARMPFTASSSHSASVGSLAISLEELLPHRT | |
| | | | AYAHQVIANARALAGLLAERGFDVAGGAFGHTDTH | |
| | | | QVWVHFPEGNTPHEWGRLLTRANIRSTSVVLPSSA | |
| | | | APGLRLGTQELTRWGMTETDMAPVADLLERLLLRG | |
| | | | DDAETVAKEVVELARAFPGVAFV | |
| Streptomyces | AFH74312.1 | 66.42 | MKESPPVPPRPSQECPMDVLEVLRRKPSLNLFPIEN | 3 |
| antibioticus | | | RLSPRAREALASDANNRYPYVEGPVSHYGDVMGL | |
| | | | GEVYDYCVDLAKEFYGARHGCVHFLSGLHTMYTVI | |
| | | | TALVPAGSRVMVLHPEDGGHYATITICEGLGHSVS | |
| | | | RLPFDRKTLLIDYEELAVQLAESPVDVIYLDASSML | |
| | | | RLPDARLLRQAAPDTLLCLDASHLMGILPAAPKTLV | |
| | | | FDGGFDTVSGSTHKTLPGPQKGLMVTNDATLAGK | |
| | | | VMERIPFTASSSHAGNVGALAITLEELMPCRVEHA | |
| | | | QQIIANARELAAQLAQRGFSVAGEEFGWTETHQV | |
| | | | WAYIPEEQGPHGWGRVLTRANVRSTTVPLPSSDG | |
| | | | LPALRLGTQELTRSGMKEAEMTEVADILERLLLRGE | |
| | | | APEQVIGTVRDLALRFPGVSWIGSADTTSVD | |
| Streptomyces | WP_003953013.1 | 77.95 | MDVLAALERKPSLNLFPIENRLSPRASAALATDAVN | 4 |
| clavuligerus | | | RYPYSETPVAVYGDVTGLAEVYAYCEDLAKRFFGAR | |
| | | | HAGVQFLSGLHTMHTVLTALTPPGGRVLVLAPEDG | |
| | | | GHYATVTICRGFGYEVEFLPFDRRTLEIDYAVLAARL | |
| | | | SRRPADVIYLDASSILRFIDARALRLAAPDALICLDA | |
| | | | SHILGLLPVAPQTLVLDGGFDSISGSTHKTFPGPQK | |
| | | | GLLVTDSDVVAEKVAARMPFTASSSHSASVGSLAI | |
| | | | SLEELLPHRTAYAHQVIANARALAGLLAERGFDVAG | |
| | | | GAFGHTDTHQVWVHFPEGNTPHEWGRLLTRANIR | |
| | | | STSVVLPSSAAPGLRLGTQELTRWGMTETDMAPVA | |
| | | | DLLERLLLRGDDAETVAKEVVELARAFPGVAFV | |
| Kitasatospora | WP_033817545.1 | 91.73 | MDVLAALERKHSLNLFPIENRLSPRAAAALASDAVN | 5 |
| sp. MBT63 | | | RYPYSETDVAVYGDVSGLNGVYDYCVSLTKEFYGA | |
| | | | RHAYVQFLSGLHTMHTVLTAVTPPGGRVMVLAPDD | |
| | | | GGHYATVTICRGFGYQVEFVPFDRQALEIDYAALAE | |
| | | | RTAEQRVDVIYLDASTVLRMPDARALRAAAPDAVL | |
| | | | CLDASHLLGLLPAAPDTLVLDGGFDSISGSTHKTLP | |
| | | | GPQKGLLVTNSDAIAEQVGARIPFTASSSHSASVG | |
| | | | SLAITLEELLPYREEYPRQVIANARELGRQLAARGFD | |
| | | | VAGGKFGHTDTHQVWVHHPEGNTPHEWGRLLTA | |
| | | | TDIRTTTVVLPSSARSGLRLGTQELTRWGMKEQD | |
| | | | MATVAELLERLLLRGEKSASVAADVQDLARSFPGV | |
| | | | AFAGRPVPLAVA | |
| Streptomyces | WP_055514611.1 | 74.94 | MDVLATLRRQPSLNLFPIENRLSPRALEALSSDANN | 6 |
| aurantiacus | | | RYPYSETDVAVYGDVTGLNDVFTYCTDLTKQFYGA | |
| | | | RHAYVNFLSGLHTMHTVITAVATAGDRVMVLAPED | |
| | | | GGHYATATICRGYGHEVDFLPFDRGTLEIDYAKLAT | |
| | | | TVAERPVDLIYLDASSMLRFPDARALRAAAPDALIC | |
| | | | LDASHLLGLLPVAPQTLVLDGGFDSISGSTHKTMP | |
| | | | GPQKGLLVTNSDRMAELVGARIPFTASSSHSASVG | |
| | | | SLAITLEELMPHRTAYAQQVIDNARALGSQLASRGF | |
| | | | DVAGKDFGYSETHQVWVHLPDGHTTHQWGRTLT | |
| | | | AAGIRSTTVQLPSTGRPGLRLGTQELTRWGMRESD | |
| | | | MSVVADLLARLLLRGEAVKEIAEDVSTLALSYPGVA | |
| | | | FAGPLAPLASR | |
| Streptomyces | WP_079663791.1 | 75.44 | MDVLATLRQKPSLNLFPIENRLSPRALEALATDANN | 7 |
| sp. 3214.6 | | | RYPYSETPVAVYGDVTGLNDVYEYCVELTKRFYGAR | |
| | | | HGFVNFLSGLHTMHTVITAVARPGDRVMLLAPEDG | |
| | | | GHYATDTICAGYGYEREFLPFDRAAMEIDYAKLAVR | |
| | | | VAERPVDLIYLDASSTLRFPDARALRAAAPDALICL | |
| | | | DASHLLGLLPVAPQTLVLDGGFDSISGSTHKTLPGP | |
| | | | QKGLLVTNSDTMADKVAARIPYTASSSHSANVGAL | |
| | | | AVTLEELLPHRAAYAQQVIANARALGRELAGRGFD | |
| | | | VAGASFGHTDTHQVWVQFPEGNTPHEWGRTLTAA | |
| | | | AIRTTTVVLPSNAQPGLRLGTQELTRWGMREQDM | |
| | | | SAVAELLARLLLRGESVESVTGDVAELALSFPGVAF | |
| | | | AGALEPVTAP | |
| Salinispora | WP_080645245.1 | 63.12 | MFPIENRLSPRAGMALSSDATNRYPYVEGALTHYG | 8 |
| pacifica | | | DVSGLNDVYAYCVDLARKYLGGRYGCVHFLSGLHT | |
| | | | MYTVITALVPPGSRIMALDPEDGGHYATVTICEGLG | |
| | | | HKMSFLPFDRERLLIDYERLADQLRQEPVDVIYVDA | |
| | | | SSMLRFPDARALRAAAPDTLLLLDASHLMGLLPAAP | |
| | | | QTGVLDGGFDIIQGSTHKTMPGPQKGLMVTNHEE | |
| | | | LVRKVEARVPYTASSSHAANVGALAITLEELLPCRL | |
| | | | SYARQVIANARELAGQLAGRGFGVAGEAFGWTDT | |
| | | | HQVWLDIPAEIGPHRWGRLLTQANVRSTTVPLPSS | |
| | | | GGLPALRLGTQELTRVGMEEQEMAEVASILDRILLR | |
| | | | GENPDSVVETVTKLVTRFPEVKFIGKPGEDESFS | |
| unclassified | WP_093638847.1 | 81.2 | MDVLAALQRRPSLNLFPIENRLSPRAAAALATDAVN | 9 |
| Streptomyces | | | RYPYSETPVAVYGDVTGLKDVYDYCADLTKEFYGA | |
| | | | RHAFVPFLSGLHTMHTVLTAVAPPGGRVMVLAPDD | |
| | | | GGHYATVTICEGFGYEVDYLPFDRQRLEIDHAALAV | |
| | | | RTAERPVDVIYLDASTALRFPDARALRAAAPGAILC | |
| | | | LDASHLLGLLPAAPQTLVLDGGFDSISGSTHKTLPG | |
| | | | PQKGLLVTNSDSLAEKMAARIPFTASSSHSATVGS | |
| | | | LAITLEELMPHRVEYAQQIIANARRLAGELAGLGFD | |
| | | | VAGEEFGHTDTHQVWVHPPEGNTPHEWGRLLTRT | |
| | | | DIRTTTVVLPSSRSSGLRLGAQELTRWGMKENDM | |
| | | | ARVAELLARLLLHHEDSGKVAADVADLARAFPGVA | |
| | | | YAGGSAAVTAG | |
| Streptomyces | WP_103501525.1 | 69.67 | MDVLAALRRRPSLNLFPIENRLSPRAREALASDAGN | 10 |
| | | | RYPYVEGPVTHYGDVMGLSEVYDYCVDLTRRFYGA | |
| | | | RFGCVHFLSGLHTMYTVITALARPGSRVMVLDPED | |
| | | | GGHYATVTICEGLGYSVSRLPFDRQRLLIDYDALAV | |
| | | | RMRERPVDLVYLDASSMLRFPDARLLRQAAPDALL | |
| | | | CLDASHLLGLLPAAPRTLVFGGGFDTISGSTHKTLP | |
| | | | GPQKGLLVTDNEALARRVRERVPFTASSSHAASVG | |
| | | | ALAITLEELMPCRVAHAEQIIANARELASQLAQRGF | |
| | | | GVAGEGFGWTETHQVWVHIPEEAGPHGWGRLLT | |
| | | | RADIRSTTVPLPSSAGLPALRLGTQELTRCGMKEDT | |
| | | | MAEVAGLLARVLLRGEAPEAVVADVRALAERFPGV | |
| | | | AYVGTPEVTVEE | |
| Streptomyces | WP_125190207.1 | 66.67 | MDVLEVLRRKPSLNLFPIENRLSPRAREALASDANN | 11 |
| sp. RP5T | | | RYPYVEGPVSHYGDVMGLGEVYDYCVDLAKEFYGA | |
| | | | RHGCVHFLSGLHTMYTVITALVPAGSRVMVLHPED | |
| | | | GGHYATITICEGLGHSVSRLPFDRKTLLIDYEELAA | |
| | | | RLAESPVDVIYLDASSMLRLPDARLLRQAAPDTLLC | |
| | | | LDASHLMGILPAAPKTLVFDGGFDTVSGSTHKTLP | |
| | | | GPQKGLMVTNDATLAGKVMERIPFTASSSHAGNV | |
| | | | GALAITLEELMPCRVEHAQQIIANARELAAQLAQRG | |
| | | | FSVAGEEFGWTETHQVWAYIPEEQGPHGWGRVLT | |
| | | | RANVRSTTVPLPSSDGLPALRLGTQELTRSGMKEA | |
| | | | EMTEVADILERLLLRGEAPEQVIGTVRDLALRFPGV | |
| | | | SWIGSADTTSVD | |
| Streptomyces | WP_148000640.1 | 65.91 | MDVLEVLRRQPSLNLFPIENRLSPRAREALSSDANN | 12 |
| sp. uw30 | | | RYPYVEGPVSHYGDVMGLDKVYDYCVELAKEFYGA | |
| | | | RYGCVHFLSGLHTMYTAITALVPPRSRVMVLHPED | |
| | | | GGHYATITVCEGLGHSISRLPFDRKNLLIDYDKLAA | |
| | | | ELEENPVDAIYLDASSMLRLPDARLLRQAAPDVLMC | |
| | | | LDASHLLGILPAAPQTLVLDGGFDTISGSTHKTLPG | |
| | | | PQKGLLVTNDEALAQKVVERIPFTASSSHAGSVGA | |
| | | | LAVTLEELLPCRVEHAEQIVSNARELAAQLAGRGFS | |
| | | | VAGEEFGWTQTHQVWAYIPEEQGPHGWGRLLTEA | |
| | | | NIRSTTVPLPSSDGLPALRLGTQELTRSGMKEADM | |
| | | | AEVAEILERILLRGEAPERVAGQVRDLALRFPGVAYI | |
| | | | GSPQGMSAD | |
| Streptomyces | WP_164262348.1 | 79.2 | MDVLAALQQRPSLNLFPIENRLSPRAAAALATDAVN | 13 |
| sp. | | | RYPYSETPVAVYGDVAGLSDVYDYCVDLTKEFYGA | |
| SID10853 | | | RHAFVQFLSGLHTMHTVLTAVTPRSGRVMVLAPED | |
| | | | GGHYATVTICESFGYRADYIPYDRKRLQIDHSALAA | |
| | | | RIAEQPVDVIYLDASTTLHFPDARALRAAAPDAIICL | |
| | | | DASHLLGLLPAAPQTLVLDGGFDSISGSTHKTLPGP | |
| | | | QKGLFVTNSDTVAEKVAARIPFTASSSHSATVGSL | |
| | | | AITLEELLPHRVDYARQTIANARRLGEELARRGFDL | |
| | | | PGEDFGYTDTHQVWVHPPEECSPHEWGRALTRAD | |
| | | | IRTTTVGLPSSGRSGLRLGSQELTRWGMKEADMA | |
| | | | AVAELLARLLLRGDDTGRVAADVADLAREFPGVAY | |
| | | | AGQPAPVTVT | |
| Streptomyces | WP_206775704.1 | 42.46 | MTPEEIIHRFGRVSPTLNLYPIENRLSDGARSLLGS | 14 |
| sp. | | | DLVSRYPRMSGPGYLYGDPSNVADLYEECAALACE | |
| DSM110735 | | | YFQVDHALVHFLSGLHAMQSMISTLSEPGERIVSL | |
| | | | GPDAGGHYATEQICRDFGHDTGLLPFDGVNLRVD | |
| | | | MDRLAEQHRAAPSRFYYVDLSTALRVPDMEQMRN | |
| | | | AVGGDALITFDASHILGLLPVLYDLPALWRQISLCT | |
| | | | ASTHKTFPGPQKAVMLSSDEKVVADMSEHLKFRV | |
| | | | SSAHTNSVGALAVTFSELMDSRRTYARAVIDNARR | |
| | | | LAELLSERGLRVVGEHFGFTETHQIWVLPPEGTQD | |
| | | | PVDWGARLQSCGIRASVVHLPAQGTSGLRLGTQE | |
| | | | LTRMGMDPAAMTEVADLTVRALGGGDPELIRKEVA | |
| | | | DLTARYATVRNDFA | |
| Streptomyces | MBJ7903826.1 | 43.34 | MSPTLNLYPIENRLSDGARSLLGSDLVSRYPRMSG | 15 |
| sp. | | | PGYLYGDPSNVADLYEECAALACEYFQVDHALVHF | |
| DSM110735 | | | LSGLHAMQSMISTLSEPGERIVSLGPDAGGHYATE | |
| | | | QICRDFGHDTGLLPFDGVNLRVDMDRLAEQHRAA | |
| | | | PSRFYYVDLSTALRVPDMEQMRNAVGGDALITFDA | |
| | | | SHILGLLPVLYDLPALWRQISLCTASTHKTFPGPQK | |
| | | | AVMLSSDEKVVADMSEHLKFRVSSAHTNSVGALA | |
| | | | VTFSELMDSRRTYARAVIDNARRLAELLSERGLRVV | |
| | | | GEHFGFTETHQIWVLPPEGTQDPVDWGARLQSCG | |
| | | | IRASVVHLPAQGTSGLRLGTQELTRMGMDPAAMTE | |
| | | | VADLTVRALGGGDPELIRKEVADLTARYATVRNDF | |
| | | | A | |

TABLE 7
PbTTA Similarity

| | | % | | |
|---|---|---|---|---|
| | Protein | Identity | | SEQ |
| | Accession | to | | ID |
| Species | No. | PbTTA | Sequence | NO |
| Parachlamydiales | MBN2478762.1 | 100% | METSLKDFETILHLINKEEIDSNDTIHMTANENI | 16 |
| bacterium | | | MSKLSKHYLKSTLSYRYHVGMFDDQKNLTVSR | |
| | | | SCLIKNSLMLRCLSPIFLLEQQAREYVKKMFFAE | |
| | | | YADFRPLSGMHTVFCILSTLTKPNDRVYVFTTE | |
| | | | SVGHAATVSLLKSLGRKVSFIPFCEKKLDIDLEK | |
| | | | LSKQILIEKPNAILFDFGTPFYPLPIREIREIVGN | |
| | | | DVKMIYDASHVLGLIAGGQFQNPLLEGCDVLIG | |
| | | | NTHKTFPGPQKGMILYKNKSLGKEIATEIFKSAI | |
| | | | SAQHTHHAIALYVTIIEMYIHGKEYANQIIKNNH | |
| | | | ALSQALINEGFKIFKRKNQFSLSHMIAITGDFPI | |
| | | | DHHVACADLHNSNISTNSRILYDFPAVRIGVQE | |
| | | | VTRKGMKEKDMVQLAKFFKEIILDRKNISSKIK | |
| | | | EFNNKFNSIEYSLDEIYEKLF | |
| Streptomyces | WP_205360601.1 | 32.06 | MTELAAAGPVRSPHRAGGRTGPAGGLLTAVHD | 17 |
| noursei | | | DVGRLTTTVNLAAFENVLSRTARAMLHGPLAD | |
| | | | RYLIGHEQERRGLDPLLRSGLLSAAYPGVDALE | |
| | | | RAASETARQLFGAAWVDFRPLSGLHATISVFAL | |
| | | | LTAPGSTVYSIAPANGGHFATQPLLESMGRDG | |
| | | | RYLPWCASAGTVDLAAFAEVWRAHPGAMVFL | |
| | | | DHGVPLAPLPVRELRAVIGDGTLLAYDASHTLG | |
| | | | LIAGGRFQDPLAEGCDLLQGNTHKSFPGAHKG | |
| | | | LVAFADAALGQGFSERLGLALVSSQQTGPTLA | |
| | | | NYVTTLEMGVHASAYTRQMLANQAALACALGE | |
| | | | SGFAVHHPPGATGPSASHVLLVEGGRQHDGA | |
| | | | DPYALAARLMHCGVMLNARPVDGRVVLRLGVQ | |
| | | | EVTRRGMRQPEMWRLAELMARAAHTEGATAT | |
| | | | ADVAGQVAALAGAFTSVRYGFDDSEAA | |
| Pseudomonas | WP_161910813.1 | 37.83 | MGNSILELLSAEEQKCRSMLHLTSYENRMSKT | 18 |
| aeruginosa | | | AEAFLSSDLGNRYHLSTPDTHNGLDPSVHIAGF | |
| | | | SCRALSAVHRLELSAIASAKKMFNAAHIEMRLV | |
| | | | SGVHATISTIASMTKPGDIVYSIAPEDGGHFAT | |
| | | | KHVAESLGRKSRYLSWDSERLNVDLEESKALF | |
| | | | AMFPPAMVFLDHGTPLFNLPVGELRDLIPSDSL | |
| | | | LVYDASHTLGLIAGGYFQHPLCEGADILQGNTH | |
| | | | KTFPGPQKAMVMFSSPELGSRYSKSVSLGLVS | |
| | | | SQHTHHSIALGVTILEMEAFGAKYAQCMLENA | |
| | | | QVLGNALIAEGLGLVSHSGKFTTSHELLINSGW | |
| | | | PDGYLSAVDRLFDANISVNGRVAFRRPTIRLGV | |
| | | | QEITRRGMGPDEMLVIAKLIAAAVQETDSAESI | |
| | | | RLRVDQLNRDFPSTLYSFDHSCSVDSGEELQN | |
| | | | AYS | |
| Gammaproteobacteria | PIR11348.1 | 34.13 | MFLNNEISEKLHKLTDLYKYDALFHSLICEEWR | 19 |
| bacterium | | | DELTLNLCAYDNILSKSARYFLQSQLGFRYRLG | |
| CG11_big_fil_ | | | EIAKAPVNADYQQKGSLLYTEKPALTQLETKAY | |
| rev_8_21_ | | | DVAVKIFSGIGADFRPLSGVHATMCSVLALTSV | |
| 14_0_20_46_ | | | NEVVYSIDPGDGGHFATRGVVEMSGRKSVYM | |
| 22 | | | PWDRERQDVDFNRLREMLNESKPTLIILEHGCP | |
| | | | QRPLNIKRLRETVGDSVFIAYDASHTLGLMAGG | |
| | | | LFQSPLLEGCDLLQANTHKSFPGPQKALYIFAN | |
| | | | SLVQERLSSALDDALVSSQHTHNLMALCISML | |
| | | | EMELWGKEYAIKMLENSAALKNELLKLGFNVLY | |
| | | | PNDHSTHIILIEFKDEFSGKAFFQRLLASGIATN | |
| | | | FRLMRDKAVIRLGTQELTRKGFEPYQMVYIADL | |
| | | | MARANEGERGSHGVASEVSELMRNSNEVHYS | |
| | | | FDDNLSINRLIQGNYDASQH | |
| Frankia | WP_084692123.1 | 35.8 | MIEIALRELVDDLRAEEGTLARTVHLTPNENVLS | 20 |
| elaeagni | | | RLARSFLSSPIGFRYHLGTISSRRALDGVVDVH | |
| | | | GLTLGYLKAVAETEQRAVGAAQGMFDAAIADL | |
| | | | RPLSGVHAMITTLSAVTEPGDTVYSIDPACGGH | |
| | | | FATRHILQRLGRVSEYLPWDLEALTIDVPRSGE | |
| | | | AFLRTQPKAVLLDHGAPLYPLPVQALRESCPSR | |
| | | | TVLIYDGSHVLGLIAGAKFQRPLADGCDILQGN | |
| | | | MHKSFPGPQKALICAREGVIGESVVDNLSRGF | |
| | | | VSSQHTHQSVAAYVTLLEMEKYGQAYAVQMLS | |
| | | | NSRSLATSLKAAGFSLVESADTPSESHQILVRT | |
| | | | DGQDESIRWVRRLLQCGISVNARRLYGHDVLR | |
| | | | VAVQEVTRLGMIESDMEHIAEIFRTALKGKTSA | |
| | | | SVLRSECISMGRRFSRVLFSFDEHFEPVE | |
| [Flexibacter] | WP_083724355.1 | 38.61 | MIEQYIETDKEIGRLVTQLVEKEELLNTHVLHLT | 21 |
| sp. ATCC | | | ANENRLSKTAREVLSSALSFRYHLGIPADYNFD | |
| 35208 | | | DIVAKPNLLFRGLPNLYRLEDMAHRCLNKHLGG | |
| | | | VVSDSRPLSGLHAMICSISSLTSPGDIVLSICPE | |
| | | | GGGHFATATLINQLGRKSVLIDYDRKTLALSLS | |
| | | | HLHQLSKEYNVKAVFLDDSAPLYAMPLKEIRDI | |
| | | | LGPDVIVIYDASHTLGLIYGQQFLHPLQDGCDV | |
| | | | IQANTHKTFPGPQKGLLHFADNTIAGKAMQTIG | |
| | | | SCLVSSQHTHHSLAFYITALEMDLHAKNYADMI | |
| | | | VANAKLLSGALEKNGFQVLTNGKSFTDTHQILF | |
| | | | NLPGHLSHYEISRKLLECHISTNAKHVYERDVV | |
| | | | RIGVQELTRLGMRGTEMEEIAGIIKLAVLDDKK | |
| | | | EIAVGMVNELNNAFQDVHYSFDNASML | |
| Flavobacterium | WP_073398358.1 | 33.66 | MNSREIEQLIKEEENNLNSFLHLTANENVISEFV | 22 |
| pectinovorum | | | SQGLSGTFSNRYHLGQIDKFSDDDITYSNGNI | |
| | | | YKGISAINKLERITSIILNNRLGGVDTDFRPLSG | |
| | | | VHAMMCTILAVTEVNDYVLTVDPATGGHFATQ | |
| | | | NIIERTGRKALTVPLNRETLTLDYDFIAKMKDRE | |
| | | | KIKMFYIDDSFAFQPINFPLLKEILGQNTIIVYDA | |
| | | | SHPFGFIFAQQFMKPILEGCDILQANTHKIFPGP | |
| | | | QKGIIHFANKALASKVKEEIGKSLVSSQHSHHT | |
| | | | LALHLAILEMDEFCEAYAEKIIKNTRYLYNSLVE | |
| | | | KGFSILEPFQKRELLTNQLYIKVPDGQNAEGIA | |
| | | | QRFYSNNISINIRRIFDQTFLRIGLQEVTRLGFN | |
| | | | EKEMDELAIIIEDVMFSRNKINISKSVENFELQE | |
| | | | RKMLFCYQVSKFSEEKLLVE | |
| Streptomyces | WP_071966917.1 | 31.5 | MTHLAVIDTARPPARPPLRTEPPHALLAAVTDD | 23 |
| cinnamoneus | | | AARLGSTVNLAAFENVLSRTARAQLAGPLADRY | |
| | | | LIGQEHERGLRHPLVRAGLLSAGYPGVDRLESA | |
| | | | AVDTLTGLLGAGWADFRPLSGLHATTCTFALLT | |
| | | | EPGELVYSIAPDNGGHFATRPLLHSLGRRCAYL | |
| | | | PWDAAAGTVDLAGLAAAWRSDPGAMVFLDHG | |
| | | | VPLVPLPVAGLRAVTGTGPLLVYDASHTLGLIVG | |
| | | | GAFQDPLGEGCDIVQGNTHKSFPGAHKGVIVF | |
| | | | ADAEAGRRFSERMGGALVSSQQTGATLANYVT | |
| | | | ALEMGVHAPAYARQMLANRAALAYALREAGFA | |
| | | | VHRPAGADAESRSHVLLVDGAGDRFGYELADD | |
| | | | LVRAGIVLNARPVEGRIRLRLGVQEVTRRGMR | |
| | | | QREMERLADLMARAARGRLPGRGRKAVTVRV | |
| | | | RTLAETFGRVHYAFDDIHESHGTTHDGTEAAP | |
| Streptomyces | WP_039639430.1 | 31.5 | MTELAAAGPVRSPHRAGGRTDPAGGLLTAVHD | 24 |
| sp. 769 | | | DVGRLTTTVNLAAFENVLSRTARAMLHGPLAD | |
| | | | RYLIGHEQERRGLDPLLRSGLLSAAYPGVDALE | |
| | | | RAASDTARQLLGAAWVDFRPLSGLHATISVFAL | |
| | | | LTAPGSTVYSIAPANGGHFATQPLLESMGRDG | |
| | | | RYLPWCASAGTVDLAAFAQVWRAHPGAMVFL | |
| | | | DHGVPLAPLPVRELRAVIGDGALLAYDASHTLG | |
| | | | LIAGGRFQDPLAEGCDLLQGNTHKSFPGAHKG | |
| | | | LVAFADAALGQGFSERLGLALVSSQQTGPTLA | |
| | | | NYVTTLEMGVHASAYTRQMLANQAALACALGE | |
| | | | SGFVVHHPPGATGPSASHVLLVEGGRQHDDA | |
| | | | DPYALAARLMHCGVMLNARPVDGRVVLRLGVQ | |
| | | | EVTRRGMRQPEMWRLAELMARAAHTEGATAT | |
| | | | AHVAGQVAALAGAFTSVRYGFDDSEAAC | |
| Leptolyngbya | NEQ47792.1 | 38.94 | MIPDKLNALINGIREEEFLSNSVLHLTANENCLS | 25 |
| sp. | | | KLASSFLSYSIGSRYALGKSSDRNAEGTWQFG | |
| SIOISBB | | | RLTYRGMPSLHHLEEEANQIAYKLFNSTYADFR | |
| | | | PLSGVHATICTISTLTKAGDLIFSLPPESGGHFA | |
| | | | SPQIIHSLGRRNSFLPWNKQKFDIDPDRLEILY | |
| | | | RQENPSAILLDYNSPLFPLNLAQIRQIVGEHIPII | |
| | | | YDASHVAGLISGGRFQQPLNDGCTVLQANTHK | |
| | | | SFPGPQKGMIHTVQPETAHQISSALSAGLISSQ | |
| | | | QTNNLIALYITLLEMHENAKAYAKNMILNSEVLA | |
| | | | HNLDKQGFKLVNRQNKPSASHILLVEVDSQKK | |
| | | | ARQWAKKLIESGISVNARRLYGKAVLRLGIQEV | |
| | | | TRRGMTTTEMAEIAILFRNAIFDKRSCEELQQE | |
| | | | VEELMSHFPHVHYSFDNLTAN | |
| Saccharothrix | NUT50161.1 | 34.61 | MTAYESKPSRLVQMLSASPLAVDYHIGSLKDH | 26 |
| sp. | | | GTDDVVTAHGLVLRGLPGVARLEAEAAGFARR | |
| | | | ALNAREVDFRPLSGVHAILATLIALTEPGDLVLS | |
| | | | ISPEHGGHFATRYLLRRIGRRSAYLPWDAEAYA | |
| | | | VDVERLAARLSARPAPAAVLFDHGLPLTRQPVE | |
| | | | RIREVVGERALVLYDASHTLGLVVGRRFQDPLG | |
| | | | EGADVVQGNTHKSFPGVQKAVIATRSEELGER | |
| | | | IGSALSDGLVSSQHTHHAVATYAAFLEMREFG | |
| | | | EGYAEAMIANARALAAELEALGARVIGPAGRW | |
| | | | TDSHEVFVAPGAGLAAATWAERLIRAGVSVNA | |
| | | | RRVHGQDALRIGVQSVTRAGMTTAEMASIARV | |
| | | | LTWFLHAERPRAHQSSLIRALTGDFSSVYCSFD | |
| | | | HSLGLSAA | |
| Deltaproteobacteria | MBF0105037.1 | 38.52 | MLSIAQKSSPVFDELKFHLEGIKKQEQQDREIL | 27 |
| bacterium | | | NLNAYDNRVSKTVLSLLSSNLSQRYDLGTPDT | |
| | | | HGCSDPAGMGEFLFKGLPHLYKFEQAAITAASL | |
| | | | MFGSVTSDFRPLSGMHGMICTLATLTEPDDVV | |
| | | | YSVECDYGGHFATHHVLKRLGRRPESIPVDINS | |
| | | | LSLDLEAFEKKVRRIPPRLVYLDVGCALYPLPIQ | |
| | | | DIRRIVGDETIIVYDASHTQGLIAGGVFQMPLA | |
| | | | EGADILQGNTHKTFPGPQKAMVHFADYKIAKK | |
| | | | LADSLTMGLVSSRHTHHSMALYVTLFEMLEFG | |
| | | | GQYARQTLKNATALGKKLKSSGIGLLERDGICT | |
| | | | QSNVLLINGKTVGGHVDACRRLYAANIATNSR | |
| | | | HAFGKEVIRIGVQELTRRGMNELEMDVIGGFIK | |
| | | | RVIVDKEDPFWIKREVMDFNSLFEDVHYSFDA | |
| | | | ALGY | |
| Rickettsiales | MBN8523064.1 | 49.05 | MNCIDSSKNLLLKLQNEEKRNTATLHMTANEN | 28 |
| bacterium | | | VMSNTASSFLSSNLSYRYYSDTYEKEDNLAEAK | |
| | | | YYAVGQAMYRGLPSVYEFELLARREANKMFHA | |
| | | | NFSDFCPLSGMNAVICILTTITKPGDKVFIFTPE | |
| | | | SLGHHATKIVLQNIGREVLFIPWDNEKLCIDIES | |
| | | | FEEEFSKNNAATIFLDLGTTFYPLPLKKIRQIVGT | |
| | | | RTKIIYDGSHVLGLIAGGQFQNPLQEGCDILIG | |
| | | | NTHKTFPGPQKAMILYKDEELGRRIGSELFKSV | |
| | | | VSSQHTHHALALYVTIIEMAAHGKLYAEQIVKN | |
| | | | AEVFSRELITQGFNIVTRKGHLPVSHMVGIKGR | |
| | | | FPQDNQFSAARLYMADISCNTKKIFGDNCIRIG | |
| | | | VQELTRRGMKEEEMRCIARFFKRIIHNEDSSAA | |
| | | | LEVQQLNNRFNKVMYSLDTEYQQYLKR | |
| Elusimicrobia | MBI3299585.1 | 40.43 | MNLAAAPPDPALAELRGLLGALKADEADYSEVV | 29 |
| bacterium | | | NLTANENTLSKTARSVLGSALGDRYFVGVWGD | |
| | | | REASDDGGAYYVDEGLLVKGMPAAAGLERLAA | |
| | | | RLANSMFHSRYCDFRPLSGMCAVTSVIAAATQ | |
| | | | ADDRFYIFAPKTLGHHASAALLTRMGRKVEFLP | |
| | | | WEASSMSVDLEALRRKVRAAPPRAVLLDYGSP | |
| | | | FYPLPTREIREIIGPEPLLVYDGSHVLGLIAGGQF | |
| | | | QDPLNEGCDILIGNTHKTFPGPQKGLILYRDAR | |
| | | | LGKEVSDVINVTTVSTQQTHQSLALFIAMVEM | |
| | | | GVHAADYAAQVLANSKAFSSALEAGGFDLLGL | |
| | | | AGRPSETHMVAVQGPFSGDNHAACGALQDIN | |
| | | | LNANSKGILGRGVIRLGVQDATRRGMKEPQMR | |
| | | | ELAALMRERLLGGRPGTPLKARARELARAFGGL | |
| | | | HYTLDEELSRP | |

TABLE 8
Amino Acid Sequences of other TTAs and SUMO-tag

| | | SEQ |
|---|---|---|
| | | ID |
| Species | Sequence | NO |
| Psuedomonas | MSNVKQQTAQIVDWLSSTLGKDHQYREDSLSLTANENYPSALVRLTSGS | 30 |
| fluorescenes | TAGAFYHCSFPFEVPAGEWHFPEPGHMNAIADQVRDLGKTLIGAQAFDW | |
| | RPNGGSTAEQALMLAACKPGEGFVHFAHRDGGHFALESLAQKMGIEIFH | |
| | LPVNPTSLLIDVAKLDEMVRRNPHIRIVILDQSFKLRWQPLAEIRSVLPDS | |
| | CTLTYDMSHDGGLIMGGVFDSPLSCGADIVHGNTHKTIPGPQKGYIGFK | |
| | SAQHPLLVDTSLWVCPHLQSNCHAEQLPPMWVAFKEMELFGRDYAAQIV | |
| | SNAKTLARHLHELGLDVTGESFGFTQTHQVHFAVGDLQKALDLCVNSLH | |
| | AGGIRSTNIEIPGKPGVHGIRLGVQAMTRRGMKEKDFEVVARFIADLYFK | |
| | KTEPAKVAQQIKEFLQAFPLAPLAYSFDNYLDEELLAAVYQGAQR | |
| Pseudomonas_ | MKQDESNVGPVIDWLAQTLGQDYKYRQDTLSLTANENYPSELVRLTSGS | 31 |
| sp._Irchel_ | TAGAFYHCSFPFPVPLGEWHFPEPGQMNEIADDLRGLAKRMMGAQAFD | |
| s3a18 | WRPNGGSPAEQALMLAACKQGEGFVHFAHRDGGHFALEQLATKMGIEIF | |
| | HLPVDPQSLLIDVAKLDDMVRRNPHIRIVILDQSFKLRWQPLAEIRAILPD | |
| | SCTLTYDMSHDGGLILGGVFDSPLACGADIAHGNTHKTIPGPQKGFIAFK | |
| | SAQHPLLVETSLWVCPHLQSNCHAELLPSMWAAFKEMEAFGPAYAHQM | |
| | VRNAKALANQLHELGLNVSGESFGFTETHQVHFAVGDLQQALSMCVDSL | |
| | HAGGIRSTNIEIPGKPGMHGIRLGVQAMTRRGMKEDDFRRVAGLIADLYF | |
| | KRTEPARVASKVKELLGDFPLAPLAYSFDQQIDESRRRLLERGIQR | |
| Burkholderia | MKQEPTGAFEVATVLNDIFLADHRYREVTLSLTANENYPSELVRVTSGST | 32 |
| stagnalis | AGAFYHVSFPFDVPDGEWHFPEPGHMHAVADKVRSLGKSLLHAQTFDW | |
| | RPNGGSAAEQALMLAACQPGDGFVHFAHGDGGHFALEALASKAGIEIFH | |
| | LPVDPDTLLIDVNRLATLVDAHPRIRIVILDQSFKLRWQPLRAIRDALPAH | |
| | CTLTYDASHDGGLVMGGWFDSPLRCGADVVHGNTHKTIAGPQKAYVAF | |
| | GSAEHPLLADTSIWVCPNIQSNCHAEQLPSIWVALKEIEAYGPAYASQVV | |
| | RNATAFARALHARGLDVSGESFGFTETHQVHFSVGTPEAALLTCRDVLHR | |
| | GGIRTTNIELPGKPGVHGIRLGVQAMTRRGMVERDFETVADFIAALCTRK | |
| | RTPEDVAPDVETFLGDFPLSPLAFSFDGGMTDALRAALRQGVMR | |
| Chitiniphilus | MTRTTPQARHVVERLNSVLGQDYRYREDCLSLTANENYPSALVRLAGSAT | 33 |
| shinanonensis | AGAFYHCSFPFEVPPGEWYFPESGRMGELAQQLNELGRSLLGAGTFDWR | |
| | PNGGSPAEQALMLAACKHGEGMVHFAHRDGGHFALENLAQKAGIDIFHL | |
| | PVDPQTLLIDVARLDELVRRNPQIRIVILDQSFKLRWQPLAAIRKVLPPSCT | |
| | LTYDTSHDGGLIMGGVFDSPLHCGADVIHGNTHKTVPGPQKGYIAFKSA | |
| | EHPLLVDTSLWLCPHLQSNCHAELLPPMWVAFKEMEAFGHDYAPQVARN | |
| | AKALAGHLHRLGFEVSGEAFGFTETHQVHFAVGDLQQALDLCMNTLHRG | |
| | GIRSTNIEIPGKPGIQGIRLGVQAMTRRGLREDDFEQVARFIADLHFRKA | |
| | DPAGVAAQVAEFLRAFPLAPLHYSFDQELDHELLQSLIGEALR | |
| Burkholderia | MTDFAQAVVNPFVDEQRKSRLVEKISNIFDSLHSDFALDNLYRASHLSLT | 34 |
| ubonensis | ASENYPSRFVRTLGAGMQGGFYEFAPPYAANPGEWYFPDSGAQSSLVEK | |
| | LASLGKQLFEANSFDWRPNGGSAAEQAVLLGTCARGDGFVHFAHKDGG | |
| | HFALEELAQKVGVSIFHLPIEEKSLLIDVDRLATLIKDNPHIKLVILDQSFKL | |
| | RWQPLLQIRQALPESVVLSYDASHDGGLIIGECLPQPLLFGADIVHGNTH | |
| | KTIPGPQKGYIAFKNVDHPAMKHVSDWVCPHLQSNSHAELIAPMYIALVE | |
| | MSLYGRSYAEQVIKNAKALAHALHAEGVRVSGESFGFTETHQVHVVVGS | |
| | ERKALELVTGTLALAGIRCNNIEIPGANGLFGLRLGVQALTRRGIKEHGMA | |
| | EVARFLVRLILKNESPTAIRNEIASFLESYPINTLHYSLDAHYYTPSGIKLME | |
| | EVIA | |
| Streptomyces | GVWAGDRVAQVLERLASDFVLDNTYREQHLSLTASENYPSKLVRMLGAG | 35 |
| (multi-species) | LQGGFYEFAPPYPAEAGEWAFPDSGANASLVGKLTGIGRQLFEAATFDW | |
| | RPNGGSVAEQAVLLGTCGRGDGFVHFAHKDGGHFALESLAGAAGVNTY | |
| | HLPMVDRTLLIDVDRLATLCAEHPEIKLVILDQSFKLRWQPLAQIRAALPE | |
| | GVFLAYDASHDGALIAGGVLPQPTLLGADAVHGNTHKTIAGPQKAYIAFR | |
| | DAEHPKLRAVSDWVCPQMQSNSHAELIAPMYVALSEVALYGHAYARQIL | |
| | ANAQALAHGLHEEGVRVSGESFGFTETHQVHVVTGSAADALRLSLGELA | |
| | QAGIRTTNIEVPGANGLHGLRLGVQAMTRRGLREPQMREVARLVAKVVL | |
| | RRAEPAAVRAEVADLLQHHPLDQLAYSFDSYVDSPAAARLLGEVFR | |
| Thermocladium | MREEEAIAALSKLRAIMDRHNNWRRRETINLIPSENVMSPLAEYFYLNDM | 36 |
| modestius | MGRYAEGTIGKRYYQGVSLVDEAEQMLVDLMSSLFSSRFTDVRPISGTV | |
| | ANMAVYHSVAGLGEKIASLPTAAGGHISHNETGAPKAFGLRVSYLPWSQ | |
| | ENFNVDVDAARRLIAEERPKLVLLGASLYLFPHPIKELADAAHEVGAVLMH | |
| | DSAHVLGLIAGHQFPNPLELGADIMTSSTHKTFPGPQGGVIFTTREDLFKE | |
| | IQRSVFPVMTSNYHLHRYASTIVTAIEMSTYGDEYAATVRSNAKALAEQL | |
| | HANGLPVVAEEHGFTATHQVAMDVSKFGGGGPIAKALEDANIIVNKNML | |
| | PWDKSPVKPSGIRMGVQEMTRMGMGKGEMAAVAELIAKVVIKGVEPSK | |
| | VKPEVVELRRGFTKVRYGFDLSTLGLNCPCLPLL | |
| Rugosimonospora | MLEIVGDHERKMASAVNLIPSENLLTPAARLAYLSDAYSRYFFDEREVFGK | 37 |
| africana | WSFQGGSIVGEVQREVLVPLVQKVTGARHVDVRGISGLNAMTVALAAFG | |
| | ARDRVTITVPPRHGGHPATAVVAGHFGHRAEALPFRDEAWWEVDLPALA | |
| | ELVARTDPALVYVDQATALVPLDLAGVIRTVKEVSPGTHVHADTSHINAF | |
| | VWSGLFGQPLDLGADSYGGSTHKTFAGPHKALLLTNDDAVSDKLTSVAV | |
| | NLVSHHHVSDVVALAIAMVEFAECGGVDYAQAVLANAAAFARALADAGP | |
| | GVQDAGGVLTRTHQVWYEPAGDPHRISERLFDAGIVVNPYNPLPSTGRL | |
| | GIRMGLNEATKLGFGEPEMAELAGLLHGVAVDRIAVAEAGERVAAMRQA | |
| | ARPAYCFSEDVVASKLRELTGASGAGVDELAAWLYR | |
| Streptomyces | MTSSDDCAASRTAPVAGRAELLALLGEIEKEQRINEAAVNLVPSENRISP | 38 |
| sp. NRRL | WAGAPLRTDFYNRYFFNDSLDPQGWQFRGGEGIGRLEKELALPALRRLG | |
| 30471 | RADHVNIRPVSGMSAMLVVLLGLGGEPGDGVVCVDAETGGHYATGRQI | |
| | AMLGRRPLPVRVVAGRVDLDALRTALTSCHVPLVYLDLQNSLWELDVAG | |
| | VAEVIARTSPRTVLHVDCSHTLGLILGGSHKNPLDLGADTTGGSTHKTFP | |
| | GPQKGVLFTRDENLSRKIRDAQFFTISSHHFAETLALALAAAEFEHFGAAY | |
| | SRQVLINARAFAHRLRERGFGVVEGGPQLTDTHQVWVRLPLEESADAFS | |
| | AQLASLGIRVNVQTELPDIPEPALRLGVSEITLNGGREPAMETLAEIFALVR | |
| | AGEATKAVDLFQVLPHEMGEPYFFTGLPQEAGLFHG | |
| Nocardia | MNTFDILEQLARYEVGTSRRLHLIASENPLDSDTRVPYMLAGTLARYAFGE | 39 |
| otitidiscaviarum | PGQPNWAWPGRETLIDLEADTAAALGALLGADHVNLRPTSGLSAMTVAL | |
| | SALAEHAGDRATVLSLAESDGGHGSTGFMARRFGLDWQRMPADPRTGV | |
| | VDLDALARQARSARGPLVLYLDAFMARFPFDLTGIRGAVGDSALIHYDGS | |
| | HPLGLIAGGRFQNPLAEGADSLGGSVHKTWPGPVGKGIIATNDSALASR | |
| | FDTHAAGWISHHHPADLAALALSTAWMEQHAGDYATAVIANAVQLADE | |
| | LADGGLSICADDRGATASHQVWVDIAPICPAPVAAQRLYDAGIVVNAIAI | |
| | PGLAEPGLRLGVQELTRWGLDRDGMTVLTWVLTQLLVHNAATAVVAPQ | |
| | MEALRTGLTLPEDRHGLEGFLRACDPQEVSVA | |
| Deltaproteobacteria | LTNNRELMDRIGYNLSQGLVSSQHTASLVALFIALHEARLTGKAFAKQVV | 40 |
| bacterium | ENARTLASRLAALGVPVLARSDGQFTDNHHFFINLTGVASAPHQMERLLR | |
| | AHLVVQRGMPFRNVDALRVGVQEVTRRGYGPGEMAQLAEWIASIVIGGA | |
| | DPEVVAPAVQAMAKRFDTIYYTGETVDGKLDLPEIAAPSAKGRWVDYRH | |
| | LGNDFAMDDTEFSEIRALGAAAGAFPNQTDSTGNVSLRSGARVFVSSSG | |
| | SYIKHLADGQVVELDAVDPSGELIDYHGAALPSSESLMHFLVYQNVPAGA | |
| | VVHTHYLLTNQEAADFDVAVIAPQEYASIALARAVAEASKRSRIVYIQKHG | |
| | LVFWGTDTADCLSQVHNFIHNRPNRRAAEAVYAS | |
| Saccharomyces | MSLQDSEVNQEAKPEVKPEVKPETHINLKVSDGSSEIFFKIKKTTPLRRLM | 41 |
| cerevisiae | EAFAKRQGKEMDSLRFLYDGIRIQADQAPEDLDMEDNDIIEAHREQIGG | |

1. A method for producing in vitro a beta-hydroxy non-standard amino acid (β-OH-nsAA), comprising incubating L-threonine, an aldehyde and an L-threonine transaldolase (TTA), wherein the TTA comprises an amino acid sequence having at least 90% identity to an amino acid sequence selected from the group consisting of SEQ IDs: 1-29, whereby a beta-hydroxy non-standard amino acid (β-OH-nsAA) is produced.

2. The method of claim 1, wherein the TTA consists of an amino acid sequence having at least 90% identity to an amino acid sequence selected from the group consisting of SEQ ID NO: 1-29.

3. The method of claim 1, wherein the TTA comprises an amino acid sequence selected from the group consisting of SEQ IDs: 1-29.

4. The method of claim 1, wherein the TTA consists of an amino acid sequence selected from the group consisting of SEQ IDs: 1-29.

5. The method of claim 1, wherein the TTA consists of the amino acid sequence of SEQ ID NO: 1.

6. The method of claim 1, wherein the TTA consists of the amino acid sequence of SEQ ID NO: 15.

7. The method of claim 1, wherein the TTA further comprises a small ubiquitin-like modifier motif (SUMO tag).

8. The method of claim 1, wherein the aldehyde is selected from the group consisting of aliphatic aldehydes, aromatic benzaldehydes, aromatic phenylacetaldehydes, aromatic cinnamaldehydes, and aldehydes derived from pyrimidine nucleosides.

9. The method of claim 1, wherein the aldehyde is selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin, protocatechualdehyde and uridine-5′-aldehyde.

10. The method of claim 1, wherein the aldehyde is selected from the group consisting of 4-nitro-benzaldehyde, 2-nitro-benzaldehyde, terephthalaldehyde, phenylacetaldehyde, 4-nitro-phenylacetaldehyde and protocatechualdehyde.

11. The method of claim 1, wherein the aldehyde is selected from the group consisting of benzaldehyde, 4-nitro-benzaldehyde, 2-nitro-benzaldehyde, 2-amino-benzaldehyde, terephthalaldehyde, 4-formyl benzaldehyde, 2-napthaldehyde, phenylacetaldehyde, 4-nitro-phenylacetaldehyde, 4-azido-benzaldehyde, vanillin and protocatechualdehyde.

12. The method of claim 1, further comprising incubating a carboxylic acid and a carboxylic acid reductase (CAR), whereby the aldehyde is generated from the carboxylic acid.

13. A method for producing a beta-hydroxy non-standard amino acid (μ-OH-nsAA) by recombinant cells, comprising:
(a) expressing a heterologous L-threonine transaldolase (TTA) by the recombinant cells, wherein the TTA comprises an amino acid sequence having at least 90% identity to an amino acid sequence of a protein selected from the group consisting of SEQ ID NOs: 1-29; and
(b) growing the recombinant cells in a medium, wherein the medium comprises L-threonine and an aldehyde, whereby a beta-hydroxy non-standard amino acid (β-OH-nsAA) is produced by the recombinant cells from the L-threonine and the aldehyde.

14. The method of claim 13, wherein the TTA consists of an amino acid sequence having at least 90% identity to an amino acid sequence of a protein selected from the group consisting of SEQ ID Nos: 1-29.

15. The method of claim 13, wherein the TTA comprises an amino acid sequence selected from the group consisting of SEQ IDs: 1-29.

16. The method of claim 13, wherein the TTA consists of an amino acid sequence selected from the group consisting of SEQ IDs: 1-29.

17. The method of claim 13, wherein the TTA consists of the amino acid sequence of SEQ ID NO: 1.

18. The method of claim 13, wherein the TTA consists of the amino acid sequence of SEQ ID NO: 15.

19. The method of claim 13, wherein the TTA further comprises a small ubiquitin-like modifier motif (SUMO tag).

20. The method of claim 13, wherein the aldehyde is selected from the group consisting of aliphatic aldehydes, aromatic benzaldehydes, aromatic phenylacetaldehydes, aromatic cinnamaldehydes, and aldehydes derived from pyrimidine nucleosides.

21-25. (canceled)
